SEQUENCE LISTING (246 sequences)

SEQ ID NO: 1 - protein, 5 aa, synthetic construct
  REGION 1..5: note = source = /note="Description of Artificial Sequence: Syntheticpeptide"
NYYWS

SEQ ID NO: 2 - protein, 16 aa, synthetic construct
  REGION 1..16: note = source = /note="Description of Artificial Sequence: Syntheticpeptide"
HIYSTGNIHYNPSLKS

SEQ ID NO: 3 - protein, 9 aa, synthetic construct
  REGION 1..9: note = source = /note="Description of Artificial Sequence: Syntheticpeptide"
DNGAALFDY

SEQ ID NO: 4 - protein, 13 aa, synthetic construct
  REGION 1..13: note = source = /note="Description of Artificial Sequence: Syntheticpeptide"
SGSSSNIGSNIVN

SEQ ID NO: 5 - protein, 7 aa, synthetic construct
  REGION 1..7: note = source = /note="Description of Artificial Sequence: Syntheticpeptide"
SNNQRPS

SEQ ID NO: 6 - protein, 11 aa, synthetic construct
  REGION 1..11: note = source = /note="Description of Artificial Sequence: Syntheticpeptide"
AAWDDSLNGPV

SEQ ID NO: 7 - protein, 5 aa, synthetic construct
  REGION 1..5: note = source = /note="Description of Artificial Sequence: Syntheticpeptide"
SYYWG

SEQ ID NO: 8 - protein, 16 aa, synthetic construct
  REGION 1..16: note = source = /note="Description of Artificial Sequence: Syntheticpeptide"
YIYYSGSTNYNPSLKS

SEQ ID NO: 9 - protein, 9 aa, synthetic construct
  REGION 1..9: note = source = /note="Description of Artificial Sequence: Syntheticpeptide"
MWEILGFDP

SEQ ID NO: 10 - protein, 13 aa, synthetic construct
  REGION 1..13: note = source = /note="Description of Artificial Sequence: Syntheticpeptide"
SGSSSNIGSNPVN

SEQ ID NO: 11 - protein, 7 aa, synthetic construct
  REGION 1..7: note = source = /note="Description of Artificial Sequence: Syntheticpeptide"
SNNQRPS

SEQ ID NO: 12 - protein, 11 aa, synthetic construct
  REGION 1..11: note = source = /note="Description of Artificial Sequence: Syntheticpeptide"
AAWDDSLNGPV

SEQ ID NO: 13 - protein, 5 aa, synthetic construct
  REGION 1..5: note = source = /note="Description of Artificial Sequence: Syntheticpeptide"
SYWMT

SEQ ID NO: 14 - protein, 17 aa, synthetic construct
  REGION 1..17: note = source = /note="Description of Artificial Sequence: Syntheticpeptide"
NIKQDGSERYYVDSVKG

SEQ ID NO: 15 - protein, 14 aa, synthetic construct
  REGION 1..14: note = source = /note="Description of Artificial Sequence: Syntheticpeptide"
EVGYNWNQGGYFDY

SEQ ID NO: 16 - protein, 16 aa, synthetic construct
  REGION 1..16: note = source = /note="Description of Artificial Sequence: Syntheticpeptide"
RSSQSLLHSDGYNYLD

SEQ ID NO: 17 - protein, 7 aa, synthetic construct
  REGION 1..7: note = source = /note="Description of Artificial Sequence: Syntheticpeptide"
LGSYRAS

SEQ ID NO: 18 - protein, 9 aa, synthetic construct
  REGION 1..9: note = source = /note="Description of Artificial Sequence: Syntheticpeptide"
MQVLQTPWT

SEQ ID NO: 19 - protein, 5 aa, synthetic construct
  REGION 1..5: note = source = /note="Description of Artificial Sequence: Syntheticpeptide"
NYYWS

SEQ ID NO: 20 - protein, 16 aa, synthetic construct
  REGION 1..16: note = source = /note="Description of Artificial Sequence: Syntheticpeptide"
HIFSTGHINYDSSLKS

SEQ ID NO: 21 - protein, 9 aa, synthetic construct
  REGION 1..9: note = source = /note="Description of Artificial Sequence: Syntheticpeptide"
DNGAALFDF

SEQ ID NO: 22 - protein, 13 aa, synthetic construct
  REGION 1..13: note = source = /note="Description of Artificial Sequence: Syntheticpeptide"
SGSSSNIGSNIVN

SEQ ID NO: 23 - protein, 7 aa, synthetic construct
  REGION 1..7: note = source = /note="Description of Artificial Sequence: Syntheticpeptide"
SDNQRPS

SEQ ID NO: 24 - protein, 11 aa, synthetic construct
  REGION 1..11: note = source = /note="Description of Artificial Sequence: Syntheticpeptide"
AAWDDSLNGPV

SEQ ID NO: 25 - protein, 5 aa, synthetic construct
  REGION 1..5: note = source = /note="Description of Artificial Sequence: Syntheticpeptide"
NYAMG

SEQ ID NO: 26 - protein, 17 aa, synthetic construct
  REGION 1..17: note = source = /note="Description of Artificial Sequence: Syntheticpeptide"
VISWSGGSTYYADSVKG

SEQ ID NO: 27 - protein, 18 aa, synthetic construct
  REGION 1..18: note = source = /note="Description of Artificial Sequence: Syntheticpeptide"
QFSGASTVVAGTALDYDY

SEQ ID NO: 28 - protein, 5 aa, synthetic construct
  REGION 1..5: note = source = /note="Description of Artificial Sequence: Syntheticpeptide"
NYGMG

SEQ ID NO: 29 - protein, 17 aa, synthetic construct
  REGION 1..17: note = source = /note="Description of Artificial Sequence: Syntheticpeptide"
GISWSGGSTDYADSVKG

SEQ ID NO: 30 - protein, 17 aa, synthetic construct
  REGION 1..17: note = source = /note="Description of Artificial Sequence: Syntheticpeptide"
VFSGAETAYYPSDDYDY

SEQ ID NO: 31 - protein, 5 aa, synthetic construct
  REGION 1..5: note = source = /note="Description of Artificial Sequence: Syntheticpeptide"
NYGMG

SEQ ID NO: 32 - protein, 17 aa, synthetic construct
  REGION 1..17: note = source = /note="Description of Artificial Sequence: Syntheticpeptide"
GITWSGGSTHYADLVKG

SEQ ID NO: 33 - protein, 17 aa, synthetic construct
  REGION 1..17: note = source = /note="Description of Artificial Sequence: Syntheticpeptide"
VFSGAETAYYPSTEYDY

SEQ ID NO: 34 - protein, 5 aa, synthetic construct
  REGION 1..5: note = source = /note="Description of Artificial Sequence: Syntheticpeptide"
NYGMG

SEQ ID NO: 35 - protein, 17 aa, synthetic construct
  REGION 1..17: note = source = /note="Description of Artificial Sequence: Syntheticpeptide"
GISWSGGSTYYADSVKG

SEQ ID NO: 36 - protein, 17 aa, synthetic construct
  REGION 1..17: note = source = /note="Description of Artificial Sequence: Syntheticpeptide"
VFSGAETAQYPSYDYDY

SEQ ID NO: 37 - protein, 5 aa, synthetic construct
  VARIANT 5: note = /replace="S"
  SITE 1..5: note = /note="Variant residues given in the sequence have nopreference with respect to those in the annotationsfor variant positions"
  REGION 1..5: note = source = /note="Description of Artificial Sequence: Syntheticpeptide"
NYAMG

SEQ ID NO: 38 - protein, 17 aa, synthetic construct
  REGION 1..17: note = source = /note="Description of Artificial Sequence: Syntheticpeptide"
AISWSGGSTSYADSVKG

SEQ ID NO: 39 - protein, 21 aa, synthetic construct
  REGION 1..21: note = source = /note="Description of Artificial Sequence: Syntheticpeptide"
QFSGADYGFGRLGIRGYEYDY

SEQ ID NO: 40 - protein, 5 aa, synthetic construct
  REGION 1..5: note = source = /note="Description of Artificial Sequence: Syntheticpeptide"
NYAMG

SEQ ID NO: 41 - protein, 17 aa, synthetic construct
  REGION 1..17: note = source = /note="Description of Artificial Sequence: Syntheticpeptide"
AISWSGGSTYYADSVKG

SEQ ID NO: 42 - protein, 21 aa, synthetic construct
  REGION 1..21: note = source = /note="Description of Artificial Sequence: Syntheticpeptide"
MFSGSESQLVVVITNLYEYDY

SEQ ID NO: 43 - protein, 5 aa, synthetic construct
  REGION 1..5: note = source = /note="Description of Artificial Sequence: Syntheticpeptide"
NYAMG

SEQ ID NO: 44 - protein, 17 aa, synthetic construct
  REGION 1..17: note = source = /note="Description of Artificial Sequence: Syntheticpeptide"
TISWSGGSTYYADSVKG

SEQ ID NO: 45 - protein, 18 aa, synthetic construct
  REGION 1..18: note = source = /note="Description of Artificial Sequence: Syntheticpeptide"
AFSGSDYANTKKEVEYDY

SEQ ID NO: 46 - protein, 5 aa, synthetic construct
  REGION 1..5: note = source = /note="Description of Artificial Sequence: Syntheticpeptide"
DYCIA

SEQ ID NO: 47 - protein, 17 aa, synthetic construct
  REGION 1..17: note = source = /note="Description of Artificial Sequence: Syntheticpeptide"
CITTSDGSTYYADSVKG

SEQ ID NO: 48 - protein, 18 aa, synthetic construct
  REGION 1..18: note = source = /note="Description of Artificial Sequence: Syntheticpeptide"
YFGYGCYGGAQDYRAMDY

SEQ ID NO: 49 - protein, 117 aa, synthetic construct
  REGION 1..117: note = source = /note="Description of Artificial Sequence: Syntheticpolypeptide"
QVQLQESGPGLVKPSETLSLTCSVSGASIRNYYWSWIRQTAGKGLEWLGHIYSTGNIHYNPSLKSRVTMSVDTSNNQFSLKLRSVTAADTAVYYCARDNGAALFDYWGQGTLVTVSS

SEQ ID NO: 50 - protein, 110 aa, synthetic construct
  REGION 1..110: note = source = /note="Description of Artificial Sequence: Syntheticpolypeptide"
QSVLTQPPSASGTPGQRVTISCSGSSSNIGSNIVNWYQQFPGTAPKLLIYSNNQRPSGVPDRVSGSKSGTSASLAISGLQSEDEADYYCAAWDDSLNGPVFGPGTKVTVL

SEQ ID NO: 51 - protein, 117 aa, synthetic construct
  REGION 1..117: note = source = /note="Description of Artificial Sequence: Syntheticpolypeptide"
QVQLQESGPGLVKPSETLSLTCTVSGGSISSYYWGWIRQPPGKGLEWIGYIYYSGSTNYNPSLKSRVTISVDTSKNQFSLKLSSVTAADTAVYYCARMWEILGFDPWGQGTLVTVSS

SEQ ID NO: 52 - protein, 110 aa, synthetic construct
  REGION 1..110: note = source = /note="Description of Artificial Sequence: Syntheticpolypeptide"
QSVLTQPPSASGTPGQRVTISCSGSSSNIGSNPVNWYQQLPGTAPKLLIYSNNQRPSGVPDRFSGSKSGTSASLAISGLQSEDEADYFCAAWDDSLNGPVFGGGTKLTVL

SEQ ID NO: 53 - protein, 123 aa, synthetic construct
  REGION 1..123: note = source = /note="Description of Artificial Sequence: Syntheticpolypeptide"
EVQLVESGGGLVQPGGSLRLSCVVSGFTFSSYWMTWVRQAPGKGLEWVANIKQDGSERYYVDSVKGRFTISRDSAKNSLYLQMNSLRAEDTAVYYCAREVGYNWNQGGYFDYWGQGTLVTVSS

SEQ ID NO: 54 - protein, 112 aa, synthetic construct
  REGION 1..112: note = source = /note="Description of Artificial Sequence: Syntheticpolypeptide"
DIVMTQSPLSLPVTPGEPASISCRSSQSLLHSDGYNYLDWYLQKSGQSPQLLIYLGSYRASGVPDRFSGSGSGTDFTLKISRVEAEDVGIYYCMQVLQTPWTFGQGTKVEIK

SEQ ID NO: 55 - protein, 117 aa, synthetic construct
  REGION 1..117: note = source = /note="Description of Artificial Sequence: Syntheticpolypeptide"
QVQLQESGPGLVKPSETLSLTCSVSGGSIRNYYWSWIRQSAGKELEWFGHIFSTGHINYDSSLKSRVTMSVDTSNNQFSLKLRSVTAADTAVYYCARDNGAALFDFWGQGTLVTVSS

SEQ ID NO: 56 - protein, 110 aa, synthetic construct
  REGION 1..110: note = source = /note="Description of Artificial Sequence: Syntheticpolypeptide"
QSVLTQPPSASGTPGQRVTISCSGSSSNIGSNIVNWYQQFPGTAPKLLLYSDNQRPSGVPDRFSGSKSGTSASLAISGLQSEDEADYYCAAWDDSLNGPVFGTGTKVTVL

SEQ ID NO: 57 - protein, 127 aa, synthetic construct
  REGION 1..127: note = source = /note="Description of Artificial Sequence: Syntheticpolypeptide"
EVQLVESGGGLVQAGGSLRLSCAASGRPFSNYAMGWFRQAPGKEREFVTVISWSGGSTYYADSVKGRFTISRDNAKNTVYLQMNSLKPEDTAVYYCAAQFSGASTVVAGTALDYDYWGQGTRVTVSS

SEQ ID NO: 58 - protein, 126 aa, synthetic construct
  REGION 1..126: note = source = /note="Description of Artificial Sequence: Syntheticpolypeptide"
EVQLVESGGGLVQAGGSLRLSCAASGRPFSNYGMGWFRQAPGKKREFVAGISWSGGSTDYADSVKGRFTISRDNAKNTVYLQMNSLKPEDTAVYYCAAVFSGAETAYYPSDDYDYWGQGTQVTVSS

SEQ ID NO: 59 - protein, 126 aa, synthetic construct
  REGION 1..126: note = source = /note="Description of Artificial Sequence: Syntheticpolypeptide"
EVQLVESGGGLVQAGGSLRLSCAASGRPFSNYGMGWFRQAPGKKREFVAGISWSGGSTDYADSVKGRLTISRDNAKNTVYLQMNSLKPEDTAVYYCAAVFSGAETAYYPSDDYDYWGQGTQVTVSS

SEQ ID NO: 60 - protein, 126 aa, synthetic construct
  REGION 1..126: note = source = /note="Description of Artificial Sequence: Syntheticpolypeptide"
EVQLVESGGGLVQAGGSLRLSCAASGRPFSNYGMGWFRQAPGKKRESVAGISWSGGSTDYADSVKGRFTISRDNAKNTVYLQMNSLKPEDTAVYYCAAVFSGAETAYYPSDDYDYWGQGTQVTVSS

SEQ ID NO: 61 - protein, 126 aa, synthetic construct
  REGION 1..126: note = source = /note="Description of Artificial Sequence: Syntheticpolypeptide"
EVQLVESGGGLVQAGGSLRLSCAVSGRPFSNYGMGWFRQAPGKEREFVAGITWSGGSTHYADLVKGRFTISRDNAKNTVHLQMNSLKPEDTAVYYCAAVFSGAETAYYPSTEYDYWGQGTQVTVSS

SEQ ID NO: 62 - protein, 126 aa, synthetic construct
  REGION 1..126: note = source = /note="Description of Artificial Sequence: Syntheticpolypeptide"
EVQLVESGGGLVQAGGSLRLSCAASGRPFNNYGMGWFRQAPGKEREFVAGISWSGGSTYYADSVKGRFTISRDNAKNTVYLQMNSLKPEDTAVYYCAAVFSGAETAQYPSYDYDYWGQGTQVTVSS

SEQ ID NO: 63 - protein, 130 aa, synthetic construct
  REGION 1..130: note = source = /note="Description of Artificial Sequence: Syntheticpolypeptide"
EVQLVESGGGLVQAGGSLRLSCAASGRPFSNYAMGWFRQAPGKEREFVAAISWSGGSTSYADSVKGRFTISRDNAKNTVYLQMNSPKPEDTAIYYCAAQFSGADYGFGRLGIRGYEYDYWGQGTQVTVSS

SEQ ID NO: 64 - protein, 130 aa, synthetic construct
  REGION 1..130: note = source = /note="Description of Artificial Sequence: Syntheticpolypeptide"
EVQLVESGGGLVQAGGSLRLSCAASGRTFSNYAMGWFRQAPGKEREFVAAISWSGGSTYYADSVKGRFTISRDNAKNTVYLQMNSLKPEDTAVYYCAAMFSGSESQLVVVITNLYEYDYWGQGTQVTVSS

SEQ ID NO: 65 - protein, 127 aa, synthetic construct
  REGION 1..127: note = source = /note="Description of Artificial Sequence: Syntheticpolypeptide"
EVQLVESGGGLVQAGGSLRLSCAASGRTFSNYAMGWFRQAPGKEREFVATISWSGGSTYYADSVKGRFTISRDNAKNTVYLQMNSLKPEDTAVYYCAAAFSGSDYANTKKEVEYDYWGQGTQVTVSS

SEQ ID NO: 66 - protein, 127 aa, synthetic construct
  REGION 1..127: note = source = /note="Description of Artificial Sequence: Syntheticpolypeptide"
EVQLVESGGGLVQAGGSLRLSCAASGFTFDDYCIAWFRQAPGKEREPVSCITTSDGSTYYADSVKGRFTISSDNAKNTVYLQMNRLKPEDTAVYYCAAYFGYGCYGGAQDYRAMDYWGKGTLVTVSS

SEQ ID NO: 67 - protein, 20 aa, synthetic construct
  REGION 1..20: note = source = /note="Description of Artificial Sequence: Syntheticpeptide"
GGSEGKSSGSGSESKSTGGS

SEQ ID NO: 68 - protein, 8 aa, synthetic construct
  REGION 1..8: note = source = /note="Description of Artificial Sequence: Syntheticpeptide"
GGGSGGGS

SEQ ID NO: 69 - protein, 12 aa, synthetic construct
  REGION 1..12: note = source = /note="Description of Artificial Sequence: Syntheticpeptide"
GGGSGGGSGGGS

SEQ ID NO: 70 - protein, 16 aa, synthetic construct
  REGION 1..16: note = source = /note="Description of Artificial Sequence: Syntheticpeptide"
GGGSGGGSGGGSGGGS

SEQ ID NO: 71 - protein, 20 aa, synthetic construct
  REGION 1..20: note = source = /note="Description of Artificial Sequence: Syntheticpeptide"
GGGSGGGSGGGSGGGSGGGS

SEQ ID NO: 72 - protein, 15 aa, synthetic construct
  REGION 1..15: note = source = /note="Description of Artificial Sequence: Syntheticpeptide"
GGGGSGGGGSGGGGS

SEQ ID NO: 73 - protein, 20 aa, synthetic construct
  REGION 1..20: note = source = /note="Description of Artificial Sequence: Syntheticpeptide"
GGGGSGGGGSGGGGSGGGGS

SEQ ID NO: 74 - protein, 25 aa, synthetic construct
  REGION 1..25: note = source = /note="Description of Artificial Sequence: Syntheticpeptide"
GGGGSGGGGSGGGGSGGGGSGGGGS

SEQ ID NO: 75 - protein, 18 aa, synthetic construct
  REGION 1..18: note = source = /note="Description of Artificial Sequence: Syntheticpeptide"
GSTSGSGKPGSGEGSTKG

SEQ ID NO: 76 - protein, 14 aa, synthetic construct
  REGION 1..14: note = source = /note="Description of Artificial Sequence: Syntheticpeptide"
IRPRAIGGSKPRVA

SEQ ID NO: 77 - protein, 15 aa, synthetic construct
  REGION 1..15: note = source = /note="Description of Artificial Sequence: Syntheticpeptide"
GKGGSGKGGSGKGGS

SEQ ID NO: 78 - protein, 15 aa, synthetic construct
  REGION 1..15: note = source = /note="Description of Artificial Sequence: Syntheticpeptide"
GGKGSGGKGSGGKGS

SEQ ID NO: 79 - protein, 15 aa, synthetic construct
  REGION 1..15: note = source = /note="Description of Artificial Sequence: Syntheticpeptide"
GGGKSGGGKSGGGKS

SEQ ID NO: 80 - protein, 15 aa, synthetic construct
  REGION 1..15: note = source = /note="Description of Artificial Sequence: Syntheticpeptide"
GKGKSGKGKSGKGKS

SEQ ID NO: 81 - protein, 15 aa, synthetic construct
  REGION 1..15: note = source = /note="Description of Artificial Sequence: Syntheticpeptide"
GGGKSGGKGSGKGGS

SEQ ID NO: 82 - protein, 15 aa, synthetic construct
  REGION 1..15: note = source = /note="Description of Artificial Sequence: Syntheticpeptide"
GKPGSGKPGSGKPGS

SEQ ID NO: 83 - protein, 20 aa, synthetic construct
  REGION 1..20: note = source = /note="Description of Artificial Sequence: Syntheticpeptide"
GKPGSGKPGSGKPGSGKPGS

SEQ ID NO: 84 - protein, 20 aa, synthetic construct
  REGION 1..20: note = source = /note="Description of Artificial Sequence: Syntheticpeptide"
GKGKSGKGKSGKGKSGKGKS

SEQ ID NO: 85 - protein, 14 aa, synthetic construct
  REGION 1..14: note = source = /note="Description of Artificial Sequence: Syntheticpeptide"
STAGDTHLGGEDFD

SEQ ID NO: 86 - protein, 15 aa, synthetic construct
  REGION 1..15: note = source = /note="Description of Artificial Sequence: Syntheticpeptide"
GEGGSGEGGSGEGGS

SEQ ID NO: 87 - protein, 15 aa, synthetic construct
  REGION 1..15: note = source = /note="Description of Artificial Sequence: Syntheticpeptide"
GGEGSGGEGSGGEGS

SEQ ID NO: 88 - protein, 15 aa, synthetic construct
  REGION 1..15: note = source = /note="Description of Artificial Sequence: Syntheticpeptide"
GEGESGEGESGEGES

SEQ ID NO: 89 - protein, 15 aa, synthetic construct
  REGION 1..15: note = source = /note="Description of Artificial Sequence: Syntheticpeptide"
GGGESGGEGSGEGGS

SEQ ID NO: 90 - protein, 20 aa, synthetic construct
  REGION 1..20: note = source = /note="Description of Artificial Sequence: Syntheticpeptide"
GEGESGEGESGEGESGEGES

SEQ ID NO: 91 - protein, 18 aa, synthetic construct
  REGION 1..18: note = source = /note="Description of Artificial Sequence: Syntheticpeptide"
GSTSGSGKPGSGEGSTKG

SEQ ID NO: 92 - protein, 19 aa, synthetic construct
  REGION 1..19: note = source = /note="Description of Artificial Sequence: Syntheticpeptide"
PRGASKSGSASQTGSAPGS

SEQ ID NO: 93 - protein, 19 aa, synthetic construct
  REGION 1..19: note = source = /note="Description of Artificial Sequence: Syntheticpeptide"
GTAAAGAGAAGGAAAGAAG

SEQ ID NO: 94 - protein, 19 aa, synthetic construct
  REGION 1..19: note = source = /note="Description of Artificial Sequence: Syntheticpeptide"
GTSGSSGSGSGGSGSGGGG

SEQ ID NO: 95 - protein, 20 aa, synthetic construct
  REGION 1..20: note = source = /note="Description of Artificial Sequence: Syntheticpeptide"
GKPGSGKPGSGKPGSGKPGS

SEQ ID NO: 96 - protein, 4 aa, synthetic construct
  REGION 1..4: note = source = /note="Description of Artificial Sequence: Syntheticpeptide"
GSGS

SEQ ID NO: 97 - protein, 10 aa, synthetic construct
  REGION 1..10: note = source = /note="Description of Artificial Sequence: Syntheticpeptide"
APAPAPAPAP

SEQ ID NO: 98 - protein, 20 aa, synthetic construct
  REGION 1..20: note = source = /note="Description of Artificial Sequence: Syntheticpeptide"
APAPAPAPAPAPAPAPAPAP

SEQ ID NO: 99 - protein, 32 aa, synthetic construct
  REGION 1..32: note = source = /note="Description of Artificial Sequence: Syntheticpolypeptide"
AEAAAKEAAAKEAAAAKEAAAAKEAAAAKAAA

SEQ ID NO: 100 - protein, 5 aa, synthetic construct
  REGION 1..5: note = source = /note="Description of Artificial Sequence: Syntheticpeptide"
GGGGS

SEQ ID NO: 101 - protein, 447 aa, synthetic construct
  VARIANT 447: note = /replace=" "
  SITE 1..447: note = /note="Variant residues given in the sequence have nopreference with respect to those in the annotationsfor variant positions"
  REGION 1..447: note = source = /note="Description of Artificial Sequence: Syntheticpolypeptide"
QVQLQESGPGLVKPSETLSLTCSVSGASIRNYYWSWIRQTAGKGLEWLGHIYSTGNIHYNPSLKSRVTMSVDTSNNQFSLKLRSVTAADTAVYYCARDNGAALFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 102 - protein, 216 aa, synthetic construct
  REGION 1..216: note = source = /note="Description of Artificial Sequence: Syntheticpolypeptide"
QSVLTQPPSASGTPGQRVTISCSGSSSNIGSNIVNWYQQFPGTAPKLLIYSNNQRPSGVPDRVSGSKSGTSASLAISGLQSEDEADYYCAAWDDSLNGPVFGPGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 103 - protein, 358 aa, synthetic construct
  VARIANT 358: note = /replace=" "
  SITE 1..358: note = /note="Variant residues given in the sequence have nopreference with respect to those in the annotationsfor variant positions"
  REGION 1..358: note = source = /note="Description of Artificial Sequence: Syntheticpolypeptide"
EVQLVESGGGLVQAGGSLRLSCAASGRPFSNYGMGWFRQAPGKKREFVAGISWSGGSTDYADSVKGRFTISRDNAKNTVYLQMNSLKPEDTAVYYCAAVFSGAETAYYPSDDYDYWGQGTQVTVSSEPKSSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 447 aa, synthetic construct; identical to SEQ ID NO: 101
  VARIANT 447: note = /replace=" "
  SITE 1..447: note = /note="Variant residues given in the sequence have nopreference with respect to those in the annotationsfor variant positions"
  REGION 1..447: note = source = /note="Description of Artificial Sequence: Syntheticpolypeptide"

SEQ ID NO: 105 - protein, 216 aa, synthetic construct; identical to SEQ ID NO: 102
  REGION 1..216: note = source = /note="Description of Artificial Sequence: Syntheticpolypeptide"

SEQ ID NO: 106 - protein, 359 aa, synthetic construct
  VARIANT 359: note = /replace=" "
  SITE 1..359: note = /note="Variant residues given in the sequence have nopreference with respect to those in the annotationsfor variant positions"
  REGION 1..359: note = source = /note="Description of Artificial Sequence: Syntheticpolypeptide"
EVQLVESGGGLVQAGGSLRLSCAASGRPFSNYAMGWFRQAPGKEREFVTVISWSGGSTYYADSVKGRFTISRDNAKNTVYLQMNSLKPEDTAVYYCAAQFSGASTVVAGTALDYDYWGQGTRVTVSSEPKSSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 447 aa, synthetic construct
  VARIANT 447: note = /replace=" "
  SITE 1..447: note = /note="Variant residues given in the sequence have nopreference with respect to those in the annotationsfor variant positions"
  REGION 1..447: note = source = /note="Description of Artificial Sequence: Syntheticpolypeptide"
QVQLQESGPGLVKPSETLSLTCTVSGGSISSYYWGWIRQPPGKGLEWIGYIYYSGSTNYNPSLKSRVTISVDTSKNQFSLKLSSVTAADTAVYYCARMWEILGFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 108 - protein, 216 aa, synthetic construct
  REGION 1..216: note = source = /note="Description of Artificial Sequence: Syntheticpolypeptide"
QSVLTQPPSASGTPGQRVTISCSGSSSNIGSNPVNWYQQLPGTAPKLLIYSNNQRPSGVPDRFSGSKSGTSASLAISGLQSEDEADYFCAAWDDSLNGPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 109 - protein, 358 aa, synthetic construct; identical to SEQ ID NO: 103
  VARIANT 358: note = /replace=" "
  SITE 1..358: note = /note="Variant residues given in the sequence have nopreference with respect to those in the annotationsfor variant positions"
  REGION 1..358: note = source = /note="Description of Artificial Sequence: Syntheticpolypeptide"

SEQ ID NO: 110 - protein, 447 aa, synthetic construct; identical to SEQ ID NO: 107
  VARIANT 447: note = /replace=" "
  SITE 1..447: note = /note="Variant residues given in the sequence have nopreference with respect to those in the annotationsfor variant positions"
  REGION 1..447: note = source = /note="Description of Artificial Sequence: Syntheticpolypeptide"

SEQ ID NO: 111 - protein, 216 aa, synthetic construct; identical to SEQ ID NO: 108
  REGION 1..216: note = source = /note="Description of Artificial Sequence: Syntheticpolypeptide"

SEQ ID NO: 112 - protein, 359 aa, synthetic construct; identical to SEQ ID NO: 106
  VARIANT 359: note = /replace=" "
  SITE 1..359: note = /note="Variant residues given in the sequence have nopreference with respect to those in the annotationsfor variant positions"
  REGION 1..359: note = source = /note="Description of Artificial Sequence: Syntheticpolypeptide"

SEQ ID NO: 113 - protein, 453 aa, synthetic construct
  VARIANT 453: note = /replace=" "
  SITE 1..453: note = /note="Variant residues given in the sequence have nopreference with respect to those in the annotationsfor variant positions"
  REGION 1..453: note = source = /note="Description of Artificial Sequence: Syntheticpolypeptide"
EVQLVESGGGLVQPGGSLRLSCVVSGFTFSSYWMTWVRQAPGKGLEWVANIKQDGSERYYVDSVKGRFTISRDSAKNSLYLQMNSLRAEDTAVYYCAREVGYNWNQGGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 114 - protein, 219 aa, synthetic construct
  REGION 1..219: note = source = /note="Description of Artificial Sequence: Syntheticpolypeptide"
DIVMTQSPLSLPVTPGEPASISCRSSQSLLHSDGYNYLDWYLQKSGQSPQLLIYLGSYRASGVPDRFSGSGSGTDFTLKISRVEAEDVGIYYCMQVLQTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 358 aa, synthetic construct; identical to SEQ ID NO: 103
  VARIANT 358: note = /replace=" "
  SITE 1..358: note = /note="Variant residues given in the sequence have nopreference with respect to those in the annotationsfor variant positions"
  REGION 1..358: note = source = /note="Description of Artificial Sequence: Syntheticpolypeptide"

SEQ ID NO: 116 - protein, 453 aa, synthetic construct; identical to SEQ ID NO: 113
  VARIANT 453: note = /replace=" "
  SITE 1..453: note = /note="Variant residues given in the sequence have nopreference with respect to those in the annotationsfor variant positions"
  REGION 1..453: note = source = /note="Description of Artificial Sequence: Syntheticpolypeptide"

SEQ ID NO: 117 - protein, 219 aa, synthetic construct; identical to SEQ ID NO: 114
  REGION 1..219: note = source = /note="Description of Artificial Sequence: Syntheticpolypeptide"

SEQ ID NO: 118 - protein, 359 aa, synthetic construct; identical to SEQ ID NO: 106
  VARIANT 359: note = /replace=" "
  SITE 1..359: note = /note="Variant residues given in the sequence have nopreference with respect to those in the annotationsfor variant positions"
  REGION 1..359: note = source = /note="Description of Artificial Sequence: Syntheticpolypeptide"

SEQ ID NO: 119 - protein, 447 aa, synthetic construct
  VARIANT 447: note = /replace=" "
  SITE 1..447: note = /note="Variant residues given in the sequence have nopreference with respect to those in the annotationsfor variant positions"
  REGION 1..447: note = source = /note="Description of Artificial Sequence: Syntheticpolypeptide"
QVQLQESGPGLVKPSETLSLTCSVSGGSIRNYYWSWIRQSAGKELEWFGHIFSTGHINYDSSLKSRVTMSVDTSNNQFSLKLRSVTAADTAVYYCARDNGAALFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 120 - protein, 216 aa, synthetic construct
  REGION 1..216: note = source = /note="Description of Artificial Sequence: Syntheticpolypeptide"
QSVLTQPPSASGTPGQRVTISCSGSSSNIGSNIVNWYQQFPGTAPKLLLYSDNQRPSGVPDRFSGSKSGTSASLAISGLQSEDEADYYCAAWDDSLNGPVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 121 - protein, 358 aa, synthetic construct; identical to SEQ ID NO: 103
  VARIANT 358: note = /replace=" "
  SITE 1..358: note = /note="Variant residues given in the sequence have nopreference with respect to those in the annotationsfor variant positions"
  REGION 1..358: note = source = /note="Description of Artificial Sequence: Syntheticpolypeptide"

SEQ ID NO: 122 - protein, 447 aa, synthetic construct; identical to SEQ ID NO: 119
  VARIANT 447: note = /replace=" "
  SITE 1..447: note = /note="Variant residues given in the sequence have nopreference with respect to those in the annotationsfor variant positions"
  REGION 1..447: note = source = /note="Description of Artificial Sequence: Syntheticpolypeptide"

SEQ ID NO: 123 - protein, 216 aa, synthetic construct; identical to SEQ ID NO: 120
  REGION 1..216: note = source = /note="Description of Artificial Sequence: Syntheticpolypeptide"

SEQ ID NO: 124 - protein, 359 aa, synthetic construct; identical to SEQ ID NO: 106
  VARIANT 359: note = /replace=" "
  SITE 1..359: note = /note="Variant residues given in the sequence have nopreference with respect to those in the annotationsfor variant positions"
  REGION 1..359: note = source = /note="Description of Artificial Sequence: Syntheticpolypeptide"

SEQ ID NO: 125 - protein, 330 aa, Homo sapiens [sequence omitted]

SEQ ID NO: 126 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 127 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 128 - protein, 232 aa, Homo sapiens
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 292 aa, Homo sapiens [sequence omitted]

SEQ ID NO: 130 - protein, 247 aa, synthetic construct
  REGION 1..247: note = source = /note="Description of Artificial Sequence: Syntheticpolypeptide"
QSVLTQPPSASGTPGQRVTISCSGSSSNIGSNIVNWYQQFPGTAPKLLIYSNNQRPSGVPDRVSGSKSGTSASLAISGLQSEDEADYYCAAWDDSLNGPVFGPGTKVTVLGGSEGKSSGSGSESKSTGGSQVQLQESGPGLVKPSETLSLTCSVSGASIRNYYWSWIRQTAGKGLEWLGHIYSTGNIHYNPSLKSRVTMSVDTSNNQFSLKLRSVTAADTAVYYCARDNGAALFDYWGQGTLVTVSS

SEQ ID NO: 131 - protein, 247 aa, synthetic construct
  REGION 1..247: note = source = /note="Description of Artificial Sequence: Syntheticpolypeptide"
QSVLTQPPSASGTPGQRVTISCSGSSSNIGSNPVNWYQQLPGTAPKLLIYSNNQRPSGVPDRFSGSKSGTSASLAISGLQSEDEADYFCAAWDDSLNGPVFGGGTKLTVLGGSEGKSSGSGSESKSTGGSQVQLQESGPGLVKPSETLSLTCTVSGGSISSYYWGWIRQPPGKGLEWIGYIYYSGSTNYNPSLKSRVTISVDTSKNQFSLKLSSVTAADTAVYYCARMWEILGFDPWGQGTLVTVSS

SEQ ID NO: 132 - protein, 255 aa, synthetic construct
  REGION 1..255: note = source = /note="Description of Artificial Sequence: Syntheticpolypeptide"
DIVMTQSPLSLPVTPGEPASISCRSSQSLLHSDGYNYLDWYLQKSGQSPQLLIYLGSYRASGVPDRFSGSGSGTDFTLKISRVEAEDVGIYYCMQVLQTPWTFGQGTKVEIKGGSEGKSSGSGSESKSTGGSEVQLVESGGGLVQPGGSLRLSCVVSGFTFSSYWMTWVRQAPGKGLEWVANIKQDGSERYYVDSVKGRFTISRDSAKNSLYLQMNSLRAEDTAVYYCAREVGYNWNQGGYFDYWGQGTLVTVSS

SEQ ID NO: 133 - protein, 247 aa, synthetic construct
  REGION 1..247: note = source = /note="Description of Artificial Sequence: Syntheticpolypeptide"
QSVLTQPPSASGTPGQRVTISCSGSSSNIGSNIVNWYQQFPGTAPKLLLYSDNQRPSGVPDRFSGSKSGTSASLAISGLQSEDEADYYCAAWDDSLNGPVFGTGTKVTVLGGSEGKSSGSGSESKSTGGSQVQLQESGPGLVKPSETLSLTCSVSGGSIRNYYWSWIRQSAGKELEWFGHIFSTGHINYDSSLKSRVTMSVDTSNNQFSLKLRSVTAADTAVYYCARDNGAALFDFWGQGTLVTVSS

SEQ ID NO: 134 - protein, 10 aa, synthetic construct
  REGION 1..10: note = source = /note="Description of Artificial Sequence: Syntheticpeptide"
GASIRNYYWS

SEQ ID NO: 135 - protein, 9 aa, synthetic construct
  REGION 1..9: note = source = /note="Description of Artificial Sequence: Syntheticpeptide"
HIYSTGNIH

SEQ ID NO: 136 - protein, 9 aa, synthetic construct
  REGION 1..9: note = source = /note="Description of Artificial Sequence: Syntheticpeptide"
DNGAALFDY

SEQ ID NO: 137 - protein, 13 aa, synthetic construct
  REGION 1..13: note = source = /note="Description of Artificial Sequence: Syntheticpeptide"
SGSSSNIGSNIVN

SEQ ID NO: 138 - protein, 7 aa, synthetic construct
  REGION 1..7: note = source = /note="Description of Artificial Sequence: Syntheticpeptide"
SNNQRPS

SEQ ID NO: 139 - protein, 11 aa, synthetic construct
  REGION 1..11: note = source = /note="Description of Artificial Sequence: Syntheticpeptide"
AAWDDSLNGPV

SEQ ID NO: 140 - protein, 7 aa, synthetic construct
  REGION 1..7: note = source = /note="Description of Artificial Sequence: Syntheticpeptide"
GASIRNY

SEQ ID NO: 141 - protein, 5 aa, synthetic construct
  REGION 1..5: note = source = /note="Description of Artificial Sequence: Syntheticpeptide"
YSTGN

SEQ ID NO: 142 - protein, 8 aa, synthetic construct
  REGION 1..8: note = source = /note="Description of Artificial Sequence: Syntheticpeptide"
DNGAALFD

SEQ ID NO: 143 - protein, 9 aa, synthetic construct
  REGION 1..9: note = source = /note="Description of Artificial Sequence: Syntheticpeptide"
SSSNIGSNI

SEQ ID NO: 144 - skipped

SEQ ID NO: 145 - protein, 8 aa, synthetic construct
  REGION 1..8: note = source = /note="Description of Artificial Sequence: Syntheticpeptide"
WDDSLNGP

SEQ ID NO: 146 - protein, 8 aa, synthetic construct
  REGION 1..8: note = source = /note="Description of Artificial Sequence: Syntheticpeptide"
GASIRNYY

SEQ ID NO: 147 - protein, 7 aa, synthetic construct
  REGION 1..7: note = source = /note="Description of Artificial Sequence: Syntheticpeptide"
IYSTGNI

SEQ ID NO: 148 - protein, 11 aa, synthetic construct
  REGION 1..11: note = source = /note="Description of Artificial Sequence: Syntheticpeptide"
ARDNGAALFDY

SEQ ID NO: 149 - protein, 8 aa, synthetic construct
  REGION 1..8: note = source = /note="Description of Artificial Sequence: Syntheticpeptide"
SSNIGSNI

SEQ ID NO: 150 - skipped

SEQ ID NO: 151 - protein, 11 aa, synthetic construct
  REGION 1..11: note = source = /note="Description of Artificial Sequence: Syntheticpeptide"
AAWDDSLNGPV

SEQ ID NO: 152 - protein, 6 aa, synthetic construct
  REGION 1..6: note = source = /note="Description of Artificial Sequence: Syntheticpeptide"
RNYYWS

SEQ ID NO: 153 - protein, 12 aa, synthetic construct
  REGION 1..12: note = source = /note="Description of Artificial Sequence: Syntheticpeptide"
WLGHIYSTGNIH

SEQ ID NO: 154 - protein, 10 aa, synthetic construct
  REGION 1..10: note = source = /note="Description of Artificial Sequence: Syntheticpeptide"
ARDNGAALFD

SEQ ID NO: 155 - protein, 9 aa, synthetic construct
  REGION 1..9: note = source = /note="Description of Artificial Sequence: Syntheticpeptide"
IGSNIVNWY

SEQ ID NO: 156 - protein, 10 aa, synthetic construct
  REGION 1..10: note = source = /note="Description of Artificial Sequence: Syntheticpeptide"
LLIYSNNQRP

SEQ ID NO: 157 - protein, 10 aa, synthetic construct
  REGION 1..10: note = source = /note="Description of Artificial Sequence: Syntheticpeptide"
AAWDDSLNGP

SEQ ID NO: 158 - protein, 10 aa, synthetic construct
  REGION 1..10: note = source = /note="Description of Artificial Sequence: Syntheticpeptide"
GGSISSYYWG

SEQ ID NO: 159 - protein, 9 aa, synthetic construct
  REGION 1..9: note = source = /note="Description of Artificial Sequence: Syntheticpeptide"
YIYYSGSTN

SEQ ID NO: 160 - protein, 9 aa, synthetic construct
  REGION 1..9: note = source = /note="Description of Artificial Sequence: Syntheticpeptide"
MWEILGFDP

SEQ ID NO: 161 - protein, 13 aa, synthetic construct
  REGION 1..13: note = source = /note="Description of Artificial Sequence: Syntheticpeptide"
SGSSSNIGSNPVN

SEQ ID NO: 162 - protein, 7 aa, synthetic construct
  REGION 1..7: note = source = /note="Description of Artificial Sequence: Syntheticpeptide"
SNNQRPS

SEQ ID NO: 163 - protein, 11 aa, synthetic construct
  REGION 1..11: note = source = /note="Description of Artificial Sequence: Syntheticpeptide"
AAWDDSLNGPV

SEQ ID NO: 164 - protein, 7 aa, synthetic construct
  REGION 1..7: note = source = /note="Description of Artificial Sequence: Syntheticpeptide"
GGSISSY

SEQ ID NO: 165 - protein, 5 aa, synthetic construct
  REGION 1..5: note = source = /note="Description of Artificial Sequence: Syntheticpeptide"
YYSGS

SEQ ID NO: 166 - protein, 8 aa, synthetic construct
  REGION 1..8: note = source = /note="Description of Artificial Sequence: Syntheticpeptide"
MWEILGFD

SEQ ID NO: 167 - protein, 9 aa, synthetic construct
  REGION 1..9: note = source = /note="Description of Artificial Sequence: Syntheticpeptide"
SSSNIGSNP

SEQ ID NO: 168 - skipped

SEQ ID NO: 169 - protein, 8 aa, synthetic construct
  REGION 1..8: note = source = /note="Description of Artificial Sequence: Syntheticpeptide"
WDDSLNGP

SEQ ID NO: 170 - protein, 8 aa, synthetic construct
  REGION 1..8: note = source = /note="Description of Artificial Sequence: Syntheticpeptide"
GGSISSYY

SEQ ID NO: 171 - protein, 7 aa, synthetic construct
  REGION 1..7: note = source = /note="Description of Artificial Sequence: Syntheticpeptide"
IYYSGST

SEQ ID NO: 172 - protein, 11 aa, synthetic construct
  REGION 1..11: note = source = /note="Description of Artificial Sequence: Syntheticpeptide"
ARMWEILGFDP

SEQ ID NO: 173 - protein, 8 aa, synthetic construct
  REGION 1..8: note = source = /note="Description of Artificial Sequence: Syntheticpeptide"
SSNIGSNP

SEQ ID NO: 174 - skipped

SEQ ID NO: 175 - protein, 11 aa, synthetic construct
  REGION 1..11: note = source = /note="Description of Artificial Sequence: Syntheticpeptide"
AAWDDSLNGPV

SEQ ID NO: 176 - protein, 6 aa, synthetic construct
  REGION 1..6: note = source = /note="Description of Artificial Sequence: Syntheticpeptide"
SSYYWG

SEQ ID NO: 177 - protein, 12 aa, synthetic construct
  REGION 1..12: note = source = /note="Description of Artificial Sequence: Syntheticpeptide"
WIGYIYYSGSTN

SEQ ID NO: 178 - protein, 10 aa, synthetic construct
  REGION 1..10: note = source = /note="Description of Artificial Sequence: Syntheticpeptide"
ARMWEILGFD

SEQ ID NO: 179 - protein, 9 aa, synthetic construct
  REGION 1..9: note = source = /note="Description of Artificial Sequence: Syntheticpeptide"
IGSNPVNWY

SEQ ID NO: 180 - protein, 10 aa, synthetic construct
  REGION 1..10: note = source = /note="Description of Artificial Sequence: Syntheticpeptide"
LLIYSNNQRP

SEQ ID NO: 181 - protein, 10 aa, synthetic construct
  REGION 1..10: note = source = /note="Description of Artificial Sequence: Syntheticpeptide"
AAWDDSLNGP

SEQ ID NO: 182 - protein, 10 aa, synthetic construct
  REGION 1..10: note = source = /note="Description of Artificial Sequence: Syntheticpeptide"
GFTFSSYWMT

SEQ ID NO: 183 - protein, 10 aa, synthetic construct
  REGION 1..10: note = source = /note="Description of Artificial Sequence: Syntheticpeptide"
NIKQDGSERY

SEQ ID NO: 184 - protein, 14 aa, synthetic construct
  REGION 1..14: note = source = /note="Description of Artificial Sequence: Syntheticpeptide"
EVGYNWNQGGYFDY

SEQ ID NO: 185 - protein, 16 aa, synthetic construct
  REGION 1..16: note = source = /note="Description of Artificial Sequence: Syntheticpeptide"
RSSQSLLHSDGYNYLD

SEQ ID NO: 186 - protein, 7 aa, synthetic construct
  REGION 1..7: note = source = /note="Description of Artificial Sequence: Syntheticpeptide"
LGSYRAS

SEQ ID NO: 187 - protein, 9 aa, synthetic construct
  REGION 1..9: note = source = /note="Description of Artificial Sequence: Syntheticpeptide"
MQVLQTPWT

SEQ ID NO: 188 - protein, 7 aa, synthetic construct
  REGION 1..7: note = source = /note="Description of Artificial Sequence: Syntheticpeptide"
GFTFSSY

SEQ ID NO: 189 - protein, 6 aa, synthetic construct
  REGION 1..6: note = source = /note="Description of Artificial Sequence: Syntheticpeptide"
KQDGSE

SEQ ID NO: 190 - protein, 13 aa, synthetic construct
  REGION 1..13: note = source = /note="Description of Artificial Sequence: Syntheticpeptide"
EVGYNWNQGGYFD

SEQ ID NO: 191 - protein, 12 aa, synthetic construct
  REGION 1..12: note = source = /note="Description of Artificial Sequence: Syntheticpeptide"
SQSLLHSDGYNY

SEQ ID NO: 192 - skipped

SEQ ID NO: 193 - protein, 6 aa, synthetic construct
  REGION 1..6: note = source = /note="Description of Artificial Sequence: Syntheticpeptide"
VLQTPW

SEQ ID NO: 194 - protein, 8 aa, synthetic construct
  REGION 1..8: note = source = /note="Description of Artificial Sequence: Syntheticpeptide"
GFTFSSYW

SEQ ID NO: 195 - protein, 8 aa, synthetic construct
  REGION 1..8: note = source = /note="Description of Artificial Sequence: Syntheticpeptide"
IKQDGSER

SEQ ID NO: 196 - protein, 16 aa, synthetic construct
  REGION 1..16: note = source = /note="Description of Artificial Sequence: Syntheticpeptide"
AREVGYNWNQGGYFDY

SEQ ID NO: 197 - protein, 11 aa, synthetic construct
  REGION 1..11: note = source = /note="Description of Artificial Sequence: Syntheticpeptide"
QSLLHSDGYNY

SEQ ID NO: 198 - skipped

SEQ ID NO: 199 - protein, 9 aa, synthetic construct
  REGION 1..9: note = source = /note="Description of Artificial Sequence: Syntheticpeptide"
MQVLQTPWT

SEQ ID NO: 200 - protein, 6 aa, synthetic construct
  REGION 1..6: note = source = /note="Description of Artificial Sequence: Syntheticpeptide"
SSYWMT

SEQ ID NO: 201 - protein, 13 aa, synthetic construct
  REGION 1..13: note = source = /note="Description of Artificial Sequence: Syntheticpeptide"
WVANIKQDGSERY

SEQ ID NO: 202 - protein, 15 aa, synthetic construct
  REGION 1..15: note = source = /note="Description of Artificial Sequence: Syntheticpeptide"
AREVGYNWNQGGYFD

SEQ ID NO: 203 - protein, 12 aa, synthetic construct
  REGION 1..12: note = source = /note="Description of Artificial Sequence: Syntheticpeptide"
LHSDGYNYLDWY

SEQ ID NO: 204 - protein, 10 aa, synthetic construct
  REGION 1..10: note = source = /note="Description of Artificial Sequence: Syntheticpeptide"
LLIYLGSYRA

SEQ ID NO: 205 - protein, 8 aa, synthetic construct
  REGION 1..8: note = source = /note="Description of Artificial Sequence: Syntheticpeptide"
MQVLQTPW

SEQ ID NO: 206 - protein, 10 aa, synthetic construct
  REGION 1..10: note = source = /note="Description of Artificial Sequence: Syntheticpeptide"
GGSIRNYYWS

SEQ ID NO: 207 - protein, 9 aa, synthetic construct
  REGION 1..9: note = source = /note="Description of Artificial Sequence: Syntheticpeptide"
HIFSTGHIN

SEQ ID NO: 208 - protein, 9 aa, synthetic construct
  REGION 1..9: note = source = /note="Description of Artificial Sequence: Syntheticpeptide"
DNGAALFDF

SEQ ID NO: 209 - protein, 13 aa, synthetic construct
  REGION 1..13: note = source = /note="Description of Artificial Sequence: Syntheticpeptide"
SGSSSNIGSNIVN

SEQ ID NO: 210 - protein, 7 aa, synthetic construct
  REGION 1..7: note = source = /note="Description of Artificial Sequence: Syntheticpeptide"
SDNQRPS

SEQ ID NO: 211 - protein, 11 aa, synthetic construct
  REGION 1..11: note = source = /note="Description of Artificial Sequence: Syntheticpeptide"
AAWDDSLNGPV

SEQ ID NO: 212 - protein, 7 aa, synthetic construct
  REGION 1..7: note = source = /note="Description of Artificial Sequence: Syntheticpeptide"
GGSIRNY

SEQ ID NO: 213 - protein, 5 aa, synthetic construct
  REGION 1..5: note = source = /note="Description of Artificial Sequence: Syntheticpeptide"
FSTGH

SEQ ID NO: 214 - protein, 8 aa, synthetic construct
  REGION 1..8: note = source = /note="Description of Artificial Sequence: Syntheticpeptide"
DNGAALFD

SEQ ID NO: 215 - protein, 9 aa, synthetic construct
  REGION 1..9: note = source = /note="Description of Artificial Sequence: Syntheticpeptide"
SSSNIGSNI

SEQ ID NO: 216 - skipped

SEQ ID NO: 217 - protein, 8 aa, synthetic construct
  REGION 1..8: note = source = /note="Description of Artificial Sequence: Syntheticpeptide"
WDDSLNGP

SEQ ID NO: 218 - protein, 8 aa, synthetic construct
  REGION 1..8: note = source = /note="Description of Artificial Sequence: Syntheticpeptide"
GGSIRNYY

SEQ ID NO: 219 - protein, 7 aa, synthetic construct
  REGION 1..7: note = source = /note="Description of Artificial Sequence: Syntheticpeptide"
IFSTGHI

SEQ ID NO: 220 - protein, 11 aa, synthetic construct
  REGION 1..11: note = source = /note="Description of Artificial Sequence: Syntheticpeptide"
ARDNGAALFDF

SEQ ID NO: 221 - protein, 8 aa, synthetic construct
  REGION 1..8: note = source = /note="Description of Artificial Sequence: Syntheticpeptide"
SSNIGSNI

SEQ ID NO: 222 - skipped

SEQ ID NO: 223 - protein, 11 aa, synthetic construct
  REGION 1..11: note = source = /note="Description of Artificial Sequence: Syntheticpeptide"
AAWDDSLNGPV

SEQ ID NO: 224 - protein, 6 aa, synthetic construct
  REGION 1..6: note = source = /note="Description of Artificial Sequence: Syntheticpeptide"
RNYYWS

SEQ ID NO: 225 - protein, 12 aa, synthetic construct
  REGION 1..12: note = source = /note="Description of Artificial Sequence: Syntheticpeptide"
WFGHIFSTGHIN

SEQ ID NO: 226 - protein, 10 aa, synthetic construct
  REGION 1..10: note = source = /note="Description of Artificial Sequence: Syntheticpeptide"
ARDNGAALFD

SEQ ID NO: 227 - protein, 9 aa, synthetic construct
  REGION 1..9: note = source = /note="Description of Artificial Sequence: Syntheticpeptide"
IGSNIVNWY

SEQ ID NO: 228 - protein, 10 aa, synthetic construct
  REGION 1..10: note = source = /note="Description of Artificial Sequence: Syntheticpeptide"
LLLYSDNQRP

SEQ ID NO: 229 - protein, 10 aa, synthetic construct
  REGION 1..10: note = source = /note="Description of Artificial Sequence: Syntheticpeptide"
AAWDDSLNGP

SEQ ID NO: 230 - protein, 851 aa, synthetic construct
  REGION 1..851: note = source = /note="Description of Artificial Sequence: Syntheticpolypeptide"
DPRVRLEVQESVTVQEGLCVLVPCTFFHPVPYHTRNSPVHGYWFREGAIVSLDSPVATNKLDQEVQEETQGRFRLLGDPSRNNCSLSIVDARRRDNGSYFFRMEKGSTKYSYKSTQLSVHVTDLTHRPQILIPGALDPDHSKNLTCSVPWACEQGTPPIFSWMSAAPTSLGLRTTHSSVLIITPRPQDHGTNLTCQVKFPGAGVTTERTIQLNVSYASQNPRTDIFLGDGSGKQGVVQGSGSGSENLYFQGVRSSSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSHHHHHH

SEQ ID NO: 231 - protein, 855 aa, synthetic construct
  REGION 1..855: note = source = /note="Description of Artificial Sequence: Syntheticpolypeptide"
DPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGSGSGSENLYFQGVRSSSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSHHHHHH

SEQ ID NO: 232 - protein, 718 aa, synthetic construct
  REGION 1..718: note = source = /note="Description of Artificial Sequence: Syntheticpolypeptide"
DPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLHHHHHH

SEQ ID NO: 233 - protein, 715 aa, synthetic construct
  REGION 1..715: note = source = /note="Description of Artificial Sequence: Syntheticpolypeptide"
VHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLHHHHHH

SEQ ID NO: 234 - protein, 135 aa, synthetic construct
  REGION 1..135: note = source = /note="Description of Artificial Sequence: Syntheticpolypeptide"
DPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRGSHHHHHH

SEQ ID NO: 235 - protein, 132 aa, synthetic construct
  REGION 1..132: note = source = /note="Description of Artificial Sequence: Syntheticpolypeptide"
VHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGSHHHHHH

SEQ ID NO: 236 - protein, 251 aa, synthetic construct
  REGION 1..251: note = source = /note="Description of Artificial Sequence: Syntheticpolypeptide"
QDLEFQLVAPESVTVEEGLCVHVPCSVFYPSIKLTLGPVTGSWLRKGVSLHEDSPVATSDPRQLVQKATQGRFQLLGDPQKHDCSLFIRDAQKNDTGMYFFRVVREPFVRYSYKKSQLSLHVTSLSRTPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGSHHHHHH

SEQ ID NO: 237 - protein, 251 aa, synthetic construct
  REGION 1..251: note = source = /note="Description of Artificial Sequence: Syntheticpolypeptide"
QDLEFQLVAPESVTVEEGLCVHVPCSVFYPSIKLTLGPVTGSWLRKGVSLHEDSPVATSDPRQLVQKATQGRFQLLGDPQKHDCSLFIRDAQKNDTGMYFFRVVREPFVRYSYKKSQLSLHVTSLSRTPQILIPGALDPDHSKNLTCSVPWACEQGTPPIFSWMSAAPTSLGLRTTHSSVLIITPRPQDHGTNLTCQVKFPGAGVTTERTIQLNVSYASQNPRTDIFLGDGSGRKARKQGVVQGSHHHHHH

SEQ ID NO: 238 - protein, 250 aa, synthetic construct
  REGION 1..250: note = source = /note="Description of Artificial Sequence: Syntheticpolypeptide"
DPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGSHHHHHH

SEQ ID NO: 239 - protein, 246 aa, synthetic construct
  REGION 1..246: note = source = /note="Description of Artificial Sequence: Syntheticpolypeptide"
DPRVRLEVQESVTVQEGLCVLVPCTFFHPVPYHTRNSPVHGYWFREGAIVSLDSPVATNKLDQEVQEETQGRFRLLGDPSRNNCSLSIVDARRRDNGSYFFRMEKGSTKYSYKSTQLSVHVTDLTHRPQILIPGALDPDHSKNLTCSVPWACEQGTPPIFSWMSAAPTSLGLRTTHSSVLIITPRPQDHGTNLTCQVKFPGAGVTTERTIQLNVSYASQNPRTDIFLGDGSGKQGVVQGSHHHHHH

SEQ ID NO: 240 - protein, 230 aa, synthetic construct
  REGION 1..230: note = source = /note="Description of Artificial Sequence: Syntheticpolypeptide"
DPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGHHHHHH

SEQ ID NO: 241 - protein, 123 aa, synthetic construct
  REGION 1..123: note = source = /note="Description of Artificial Sequence: Syntheticpolypeptide"
EVQLVESGGGLVQPGGSLKVSCEASGFTFSVSAIHWVRQASGKGLEWIGRIRSKGNSYATAYAASVKGRFTISRDDSKNTAYLQMDSLKTEDTAVYYCTRHNDKWNYYGLDVWGQGTTVTVSS

SEQ ID NO: 242 - protein, 112 aa, synthetic construct
  REGION 1..112: note = source = /note="Description of Artificial Sequence: Syntheticpolypeptide"
DIVMTQSPLSLPVTPGEPASISCKSSQSLLFSNGYKFLDWYLQRPGQSPQLLIYLGSYRASGVPDRFSGSGSGTDFTLKISRVEAEDVGLYYCMQALQTPPTFGGGTKVEIK

SEQ ID NO: 243 - protein, 114 aa, synthetic construct
  REGION 1..114: note = source = /note="Description of Artificial Sequence: Syntheticpolypeptide"
QVQLVESGGGLVQPGGSLRLSCAASGITFSNYWMSWVRQAPGKGLEWVASIKRDGSDKYYVDSVKGRFTISRDNAKNSLSLQMHSLRAEDTAVYYCAKGEFDYWGQGTLVTVSS

SEQ ID NO: 244 - protein, 112 aa, synthetic construct
  REGION 1..112: note = source = /note="Description of Artificial Sequence: Syntheticpolypeptide"
DVVMTQSPLSLPVTLGQPASISCRSSQSLVYSDGNTYLNWFQQRPGQSPRRLIYKVSTRDSGVPDRFSGSGSGTDFTLKISRVEAEDVAVYYCLQGTHWPWTFGQGTKVEIK

SEQ ID NO: 245 - protein, 7 aa, synthetic construct
  REGION 1..7: note = source = /note="Description of Artificial Sequence: Syntheticpeptide"
AAAEPEA

SEQ ID NO: 246 - protein, 6 aa, synthetic construct
  REGION 1..6: note = source = /note="Description of Artificial Sequence: Synthetic6xHis tag"
HHHHHH

(19) United States
(12) Patent Application Publication
(10) Pub. No.: US 2024/0409634 A1
(43) Pub. Date: Dec. 12, 2024

(54) CD33 x Vd2 MULTISPECIFIC ANTIBODIES FOR THE TREATMENT OF CANCER

(71) Applicant: Janssen Biotech, Inc., Horsham, PA (US)

(72) Inventors: Patrick John DOONAN, Lansdale, PA (US); Sherry Lynn LA PORTE, Horsham, PA (US); Sanjaya SINGH, Blue Bell, PA (US); Paul Willem Henri lda PARREN, Odijk (NL); Sabrina Julia Louisa MERAT, s'Hertogenbosh (NL); Robertus Cornelis ROOVERS, Bunnik (NL); Sara Mohamed A ELASHKAR, Edegem (BE); Ulrike PHILIPPAR, Antwerpen (BE)

(21) Appl. No.: 18/691,254

(22) PCT Filed: Sep. 12, 2022
(86) PCT No.: PCT/IB2022/058572

§ 371 Date: Mar. 12, 2024

Related U.S. Application Data
(60) Provisional application No. 63/243,493, filed on Sep. 13, 2021.

(30) Foreign Application Priority Data
Oct. 14, 2021 (EP) 21202685.0

(51) Int. Cl. C07K 16/28; A61P 35/02

(52) U.S. Cl. CPC C07K 16/2809; A61P 35/02; C07K 2317/31; C07K 2317/52; C07K 2317/55; C07K 2317/565; C07K 2317/569; C07K 2317/622; C07K 2317/73; C07K 2317/92

(57) ABSTRACT

The invention relates to multispecific antibodies and pharmaceutical compositions comprising said antibodies, to processes for the preparation of said antibodies and to the use of said antibodies targeting CD33 and to their use in the treatment of diseases, e.g., cancer.

### SEQUENCE LISTING

The instant application contains a Sequence Listing which has been submitted electronically in ASCII format and is hereby incorporated by reference in its entirety. Said ASCII copy, created on Mar. 8, 2022, is named SEQLTXT-1.txt and is 212 kilobytes in size.

### FIELD OF THE INVENTION

The invention relates to multispecific antibodies and pharmaceutical compositions comprising said antibodies, to processes for the preparation of said antibodies and to the use of said antibodies targeting CD33 and to their use in the treatment of diseases, e.g., cancer.

### BACKGROUND OF THE INVENTION

Targeted immunotherapy in acute myeloid leukemia (AML) remains a substantial clinical challenge due to the heterogenous nature of the AML blasts and the lack of AML-specific antigens. Several immunotherapies targeting CD33 are currently undergoing phase 1 or phase 2 clinical trials in AML such as CD33×CD3 T-cell redirecting molecules and multiple autologous or allogeneic CAR T or NK cell therapies.

Vγ9Vδ2 T-cells represent an interesting subset of T-cells to explore for tumor cell immunotherapy. They represent 1-5% of the circulating T-cell population and are prevalent in a broad set of cancers, which they infiltrate independent of mutational load. These T cells sense phospho-antigen-mediated conformational changes in the butyrophilin (BTN) family of ligands on target cells and efficiently kill these cells. Vγ9Vδ2 T-cells are less affected by inhibition by PDL1 on tumor cells and the Vγ9Vδ2 T-cell population does not contain Tregs. This is relevant as the activation of Tregs by CD3 bispecific T-cell engagers has been shown to limit the activity of the latter. In addition, differential expression of phosphoantigens-activated butyrophilin (BTN3A, CD227) may contribute to greater anti-tumor activity of γδ T-cells towards cancer cells over normal cells.

### SUMMARY OF THE INVENTION

An objective of the present invention is to provide multispecific or bispecific antibodies which are capable of CD33-dependent γδ T-cell redirection.

An objective of the present invention is to provide multispecific or bispecific antibodies comprised of different CD33 binders paired with either high or low affinity Vδ2 binders.

An objective of the present invention is to provide multispecific or bispecific antibodies that are cytotoxic on CD33+ cancer cells and primary AML blast in conjunction with Vγ9Vδ2 T cells.

An objective of the present invention is to provide multispecific or bispecific antibodies that induce potent and selective T-cell mediated cytotoxicity in different cell lines.

An objective of the present invention is to provide multispecific or bispecific antibodies that present advantages with regards to safety and efficacy compared to the existing therapies.

An objective of the present invention is to provide multispecific or bispecific antibodies that induce target-dependent degranulation, activation and proliferation of the Vγ9Vδ2 T-cells.

An objective of the present invention is to provide multispecific or bispecific antibodies that show preferential killing of THP-1 cancer cells over healthy CD14+ cells (monocytes).

### DESCRIPTION OF THE FIGURES

FIG. 1: CD33xVδ2 bispecific antibodies bind to CD33-expressing cells. THP-1 cells were incubated with various concentrations of CD33xVδ2 bispecific antibodies or a negative control antibody (RSV(B21M) xVδ2 bispecific antibody). Binding was detected by FACS using an Alexa Fluor® 647 conjugated F(ab′)2 Goat anti-human IgG (H+L) (Jackson). (A) CD33xVδ2 bispecific antibodies containing JL2, JL3, JL5, or JL6 as the CD33 binding domain and 6H4 as the Vδ2 binding domain. (B) CD33xVδ2 bispecific antibodies containing JL2, JL3, JL5 or JL6 as the CD33 binding domain and 5D3 as the Vδ2 binding domain.

FIG. 2: CD33xVδ2 bispecific antibodies bind to Vγ9Vδ2 T cells. Polyclonal Vγ9Vδ2 T cells isolated and expanded from a healthy donor were incubated with various concentrations of CD33xVδ2 bispecific antibodies, a RSV(B21M) xVδ2 bispecific antibody or a negative control antibody (LAVA-188, bispecific antibody directed against two irrelevant targets). Binding was detected by FACS using an Alexa Fluor® 647 conjugated F(ab′)2 Goat anti-human IgG (H+L) (Jackson). (A) CD33xVδ2 bispecific antibodies containing JL2, JL3, JL5, or JL6 as the CD33 binding domain and 6H4 as the Vδ2 binding domain and LAVA-188. (B) CD33xVδ2 bispecific antibodies containing JL2, JL3, JL5 or JL6 the CD33 binding domain and 5D3 as the Vδ2 binding domain and LAVA-188.

FIG. 3: CD33xVδ2 bispecific antibodies preferentially mediate killing of tumor cells over healthy CD14+ cells. THP-1 target cells (panel A) or CD14+ target cells (panel B) were incubated with various concentrations of CD33xVδ2 bispecific antibodies (JL3x6H4, JL5x6H4, JL6x6H4 or JL5x5D3) or negative control antibodies (RSV(B21M)x6H4 or RSV(B21M)x5D3). Donor PBMCs (effector cells) were added at a 5:1 effector cell to target cell ratio and, after incubation, specific lysis was determined.

FIG. 4: CD33xVδ2 bispecific antibodies induce proliferation of Vγ9Vδ2 T cells. Vγ9Vδ2 T cells (effector cells) and THP-1 cells (target cells) were incubated with 1 nM of CD33xVδ2 bispecific antibodies (JL3x6H4, JL5x6H4, JL6x6H4 or JL5x5D3) at a 1:20 effector cell to target cell ratio. The fold increase of the number of Vγ9Vδ2 T cells was determined after 1, 4, 7, 11 and 14 days of incubation. Panels A and B represent Vγ9Vδ2 T cells from two different donors. Negative controls were antibodies RSV(B21M)x6H4 and RSV(B21M)x5D3 and medium.

### DETAILED DESCRIPTION OF THE INVENTION

### Definitions

The terms “first” and “second” antigen-binding regions when used herein do not refer to their orientation/position in the antibody, i.e., they have no meaning with regard to the N- or C-terminus. The terms “first” and “second” only serve to refer correctly and consistently to the two different antigen-binding regions in the claims and the description.

The term “CD33” when used herein, refers to human CD33, the sequence of which is set forth in UniProtKB-P20138.

The term “human Vδ2”, when used herein, refers to the rearranged 82 chain of the Vγ9Vδ2-T cell receptor (TCR). GenBank: CAA51166.1, gives an example of a 82 sequence. TRDV2, T cell receptor delta variable 2, represents the variable region (UniProtKB-A0JD36 (A0JD36_HUMAN) gives an example of a TRDV2 sequence). “binding the Vδ2 chain of a Vγ9Vδ2-TCR” means that the antibody can bind the δ2 chain as a separate molecule and/or as part of a Vγ9Vδ2-TCR (T cell Receptor). However, the antibody will not bind to the γ9 chain as a separate molecule.

The term “human Vγ9”, when used herein, refers to the rearranged γ9 chain of the Vγ9Vδ2-T cell receptor (TCR). GenBank: NG_001336.2 gives an example of a γ9 sequence. TRGV9, T cell receptor gamma variable 9 represents the variable region (UniProtKB-Q99603_HUMAN gives an example of a TRGV9 sequence).

The Fc (Fragment crystallizable) region of an immunoglobulin is defined as the fragment of an antibody, which would be typically generated after digestion of an antibody with papain, and which includes the two CH2-CH3 regions of an immunoglobulin and a connecting region, e.g., a hinge region. The constant domain of an antibody heavy chain defines the antibody isotype, e.g., IgG1, IgG2, IgG3, IgG4, IgA1, IgA2, IgM, IgD, or IgE. The Fc-region mediates the effector functions of antibodies with cell surface receptors called Fc receptors and proteins of the complement system.

The term “hinge region” as used herein is intended to refer to the hinge region of an immunoglobulin heavy chain. Thus, for example the hinge region of a human IgG1 antibody corresponds to amino acids 216-230 according to the EU numbering.

The term “CH2 region” or “CH2 domain” as used herein is intended to refer to the CH2 region of an immunoglobulin heavy chain. Thus, for example the CH2 region of a human IgG1 antibody corresponds to amino acids 231-340 according to the EU numbering. However, the CH2 region may also be of any of the other antibody isotypes as described herein.

The term “CH3 region” or “CH3 domain” as used herein is intended to refer to the CH3 region of an immunoglobulin heavy chain. Thus, for example the CH3 region of a human IgG1 antibody corresponds to amino acids 341-447 according to the EU numbering. However, the CH3 region may also be of any of the other antibody isotypes as described herein.

In some embodiments, however, the Fc region of the antibody has been modified to become inert; “inert” means an Fc region which a minimal or no ability to bind any Fcgamma Receptors, induce Fc-mediated cross-linking of FcRs, or induce FcR-mediated cross-linking of target antigens via two Fc regions of individual antibodies. The inert Fc region may in addition not be able to bind C1q.

The term “isotype” as used herein, refers to the immunoglobulin (sub)class (for instance IgG1, IgG2, IgG3, IgG4, IgD, IgA, IgE, IgM, or any allotype thereof, such as those allotypes of Table 2 below) that is encoded by heavy chain constant region genes. Each heavy chain isotype can be combined with either a kappa (κ) or lambda (λ) light chain. An antibody of the invention can possess any isotype.

In the context of the present invention, “competition” or “able to compete” or “competes” refers to any detectably significant reduction in the propensity for a particular binding molecule (e.g., an antibody) to bind a particular binding partner (e.g., the target) in the presence of another molecule (e.g., a different antibody binding the same target) that binds the binding partner. Typically, competition means an at least about 25 percent reduction, such as an at least about 50 percent, e.g., an at least about 75 percent, such as an at least 90 percent reduction in binding, caused by the presence of another molecule, such as an antibody, as determined by, e.g., ELISA analysis or flow cytometry using sufficient amounts of the two or more competing molecules, e.g., antibodies. Additional methods for determining binding specificity by competitive inhibition may be found in for instance Harlow et al., Antibodies: A Laboratory Manual, Cold Spring Harbor Laboratory Press, Cold Spring Harbor, N.Y., 1988), Colligan et al., eds., Current Protocols in Immunology, Greene Publishing Assoc, and Wiley InterScience N. Y., (1992, 1993), and Muller, Meth. Enzymol. 92, 589-601 (1983)).

As used in this specification and the appended claims, the singular forms “a,” “an,” and “the” include plural referents unless the content clearly dictates otherwise. Thus, for example, reference to “a cell” includes a combination of two or more cells, and the like.

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents) also provide as embodiments those independently described in terms of “consisting of” and “consisting essentially of.”

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

“Activation” or “stimulation” or “activated” or “stimulated” refers to induction of a change in the biologic state of a cell resulting in expression of activation markers, cytokine production, proliferation or mediating cytotoxicity of target cells. Cells may be activated by primary stimulatory signals. Co-stimulatory signals can amplify the magnitude of the primary signals and suppress cell death following initial stimulation resulting in a more durable activation state and thus a higher cytotoxic capacity.

“Alternative scaffold” refers to a single chain protein framework that contains a structured core associated with variable domains of high conformational tolerance. The variable domains tolerate variation to be introduced without compromising scaffold integrity, and hence the variable domains can be engineered and selected for binding to a specific antigen.

“Antigen” refers to any molecule (e.g., protein, peptide, polysaccharide, glycoprotein, glycolipid, nucleic acid, portions thereof, or combinations thereof) capable of being bound by an antigen binding domain or a T-cell receptor that is capable of mediating an immune response. Exemplary immune responses include antibody production and activation of immune cells, such as T cells, B cells or NK cells. Antigens may be expressed by genes, synthetized, or purified from biological samples such as a tissue sample, a tumor sample, a cell or a fluid with other biological components, organisms, subunits of proteins/antigens, killed or inactivated whole cells or lysates.

“Antigen binding region” or “antigen binding domain” or “antigen binding site” refers to a portion of the antibody that binds an antigen. Antigen binding regions may be synthetic, enzymatically obtainable or genetically engineered polypeptides and include portions of an immunoglobulin that bind an antigen, such as VH, the VL, the VH and the VL, Fab, Fab′, F(ab′)₂, Fd and Fv fragments, single-domain antibodies (dAb) consisting of one VH domain or one VL domain, shark variable IgNAR domains, camelized VH domains, VHH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3, alternative scaffolds that bind an antigen, and multispecific proteins comprising the antigen binding regions. Antigen binding domains (such as VH and VL) may be linked together via a synthetic linker to form various types of single antibody designs where the VH/VL domains may pair intramolecularly, or intermolecularly in those cases when the VH and VL domains are expressed by separate single chains, to form a monovalent antigen binding domain, such as single chain Fv (scFv) or diabody. Antigen binding regions may also be conjugated to other antibodies, proteins, antigen binding fragments or alternative scaffolds which may be monospecific or multispecific to engineer bispecific and multispecific proteins.

“Antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity. A heavy chain is comprised of a heavy chain variable region (VH) and a heavy chain constant region (comprised of domains CH1, hinge, CH2 and CH3). A light chain, if present, is comprised of a light chain variable region (VL) and a light chain constant region (CL). The VH and the VL regions may be further subdivided into regions of hypervariability, termed complementarity determining regions (CDR), interspersed with framework regions (FR). A VH or VL is 30 composed of three CDRs and four FR segments, arranged from amino-to-carboxy-terminus in the following order: FR1, CDR1, FR2, CDR2, FR3, CDR3 and FR4. Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

“Bispecific” refers to a molecule (such as an antibody) that specifically binds two distinct antigens or two distinct epitopes within the same antigen. The bispecific molecule may have cross-reactivity to other related antigens, for example to the same antigen from other species (homologs), such as human, monkey, or ape, for example Macaca fascicularis (cynomolgus monkey, cyno) or Pan troglodytes, or may bind an epitope that is shared between two or more distinct antigens.

Examples of different classes of bispecific antibodies include but are not limited to (i) IgG-like molecules with complementary CH3 domains to force heterodimerization; (ii) recombinant IgG-like dual targeting molecules, wherein the two sides of the molecule each contain the Fab fragment or part of the Fab fragment of at least two different antibodies; (iii) IgG fusion molecules, wherein full length IgG antibodies are fused to extra Fab fragment or parts of Fab fragment; (iv) Fc fusion molecules, wherein single chain Fv molecules or stabilized diabodies are fused to heavy-chain constant-domains, Fc-regions or parts thereof; (v) Fab fusion molecules, wherein different Fab-fragments are fused together, fused to heavy-chain constant-domains, Fc-regions or parts thereof; and (vi) scFv- and diabody-based and heavy chain antibodies (e.g., domain antibodies, Nanobodies®) wherein different single chain Fv molecules or different diabodies or different heavy-chain antibodies (e.g. domain antibodies, Nanobodies®) are fused to each other or to another protein or carrier molecule fused to heavy-chain constant-domains, Fc-regions or parts thereof.

Examples of IgG-like molecules with complementary CH3 domains molecules include but are not limited to the Triomab® (Trion Pharma/Fresenius Biotech), the Knobs-into-Holes (Genentech), CrossMAbs (Roche) and the electrostatically-matched (Amgen, Chugai, Oncomed), the LUZ-Y (Genentech, Wranik et al. J. Biol. Chem. 2012, 287 (52): 43331-9, doi: 10.1074/jbc.M112.397869. Epub 2012 Nov. 1), DIG-body and PIG-body (Pharmabcine, WO2010134666, WO2014081202), the Strand Exchange Engineered Domain body (SEEDbody) (EMD Serono), the Biclonics (Merus, WO2013157953), FcΔAdp (Regeneron), bispecific IgG1 and IgG2 (Pfizer/Rinat), Azymetric scaffold (Zymeworks/Merck,), mAb-Fv (Xencor), bivalent bispecific antibodies (Roche, WO2009080254) and DuoBody® molecules (Genmab).

Examples of recombinant IgG-like dual targeting molecules include, but are not limited, to Dual Targeting (DT)-Ig (GSK/Domantis, WO2009058383), Two-in-one Antibody (Genentech, Bostrom, et al 2009. Science 323, 1610-1614), Cross-linked Mabs (Karmanos Cancer Center), mAb2 (F-Star), Zybodies™ (Zyngenia, LaFleur et al. MAbs. 2013 March-April; 5 (2): 208-18), approaches with common light chain, KÂBodies (NovImmune, WO2012023053) and CovX-Body® (CovX/Pfizer, Doppalapudi, V. R., et al 2007. Bioorg. Med. Chem. Lett. 17, 501-506).

Examples of IgG fusion molecules include but are not limited to Dual Variable Domain (DVD)-Ig (Abbott), Dual domain double head antibodies (Unilever; Sanofi Aventis), IgG-like Bispecific (ImClone/Eli Lilly, Lewis et al. Nat Biotechnol. 2014 February; 32 (2): 191-8), Ts2Ab (MedImmune/AZ, Dimasi et al. J Mol Biol. 2009 Oct. 30; 393 (3): 672-92) and BsAb (Zymogenetics, WO2010111625), HERCULES (Biogen Idec), scFv fusion (Novartis), scFv fusion (Changzhou Adam Biotech Inc) and TvAb (Roche).

Examples of Fc fusion molecules include but are not limited to scFv/Fc Fusions (Academic Institution, Pearce et al Biochem Mol Biol Int. 1997 September; 42 (6): 1179), SCORPION (Emergent BioSolutions/Trubion, Blankenship J W, et al. AACR 100th Annual meeting 2009 (Abstract #5465); Zymogenetics/BMS, WO2010111625), Dual Affinity Retargeting Technology (Fc-DART™) (MacroGenics) and Dual (ScFv) 2-Fab (National Research Center for Antibody Medicine—China).

Examples of Fab fusion bispecific antibodies include but are not limited to F (ab) 2 (Medarex/AMGEN), Dual-Action or Bis-Fab (Genentech), Dock-and-Lock® (DNL) (ImmunoMedics), Bivalent Bispecific (Biotecnoland Fab-Fv (UCB-Celltech).

Examples of scFv-, diabody-based and domain antibodies include but are not limited to Bispecific T Cell Engager (BiTE®) (Micromet, Tandem Diabody (Tandab) (Affimed), Dual Affinity Retargeting Technology (DART™) (MacroGenics), Single-chain Diabody (Academic, Lawrence FEBS Lett. 1998 Apr. 3; 425 (3): 479-84), TCR-like Antibodies (AIT, ReceptorLogics), Human Serum Albumin ScFv Fusion (Merrimack, WO2010059315) and COMBODY molecules (Epigen Biotech, Zhu et al. Immunol Cell Biol. 2010 August; 88 (6): 667-75), dual targeting Nanobodies® (Ablynx, Hmila et al., FASEB J. 2010), dual targeting heavy chain only domain antibodies. The multispecific antibody of the invention may be in a VHH-Fc format, i.e., the antibody comprises two or more single-domain antigen-binding regions that are linked to each other via a human Fc region dimer. In this format, each single-domain antigen-binding region is fused to an Fc region polypeptide and the two fusion polypeptides form a dimeric bispecific antibody via disulfide bridges in the hinge region. Such constructs typically do not contain full, or any, CH1 or light chain sequences. FIG. 12B of WO06064136 provides an illustration of an example of this format.

Bispecific antibodies may also be of mixed format. For example, one antigen-binding region may be a Fab or scFv format and the other antigen-binding region may be comprised of or consist of a single-domain antibody. Such constructs may additionally comprise an Fc region and which, through the hinge region, links the Fc polypeptides.

“Complementarity determining regions” (CDR) are antibody regions that bind an antigen. There are three CDRs in the VH (HCDR1, HCDR2, HCDR3) and three CDRs in the VL, if present, (LCDR1, LCDR2, LCDR3). CDRs may be defined using various delineations such as Kabat (Wu et al. (1970) J Exp Med 132:211-50; Kabat et al., Sequences of Proteins of Immunological Interest, 5th Ed. Public Health Service, National Institutes of Health, Bethesda, Md., 1991), Chothia (Chothia et al. (1987) J Mol Biol 196:901-17), IMGT (Lefranc et al. (2003) Dev Comp Immunol 27:55-77), AbM (Martin and Thornton J Bmol Biol 263:800-15, 1996), or Contact, which is based on an analysis of the available complex crystal structures (MacCallum, R. M., Martin, A. C. R. and Thornton, J. T. “Antibody-antigen interactions: Contact analysis and binding site topography” J. Mol. Biol. 262:732-745). The correspondence between the various delineations and variable region numbering is described (see e.g. Lefranc et al. (2003) Dev Comp Immunol 27:55-77; Honegger and Pluckthun, J Mol Biol (2001) 309:657-70; International ImMunoGeneTics (IMGT) database; Web resources, www.imgt.org). Available programs such as ab Ysis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT, AbM, or Contact, unless otherwise explicitly stated in the specification. The CDRs of the sequences with SEQ ID NO. 1 to 48 are defined according to Kabat, supra.

“Expression vector” refers to a vector that can be utilized in a biological system or in a reconstituted biological system to direct the translation of a polypeptide encoded by a polynucleotide sequence present in the expression vector.

“Single-domain antibody”, “dAb”, “VHH” or “dAb fragment” refers to an antibody fragment composed of a VH domain (Ward et al., Nature 341:544 546 (1989)). The second antigen-binding region of the present invention may be a single-domain antibody. Single-domain antibodies (sdAb, also called Nanobody®, or VHH) are well known to the skilled person, see e.g., Hamers-Casterman et al. (1993) Nature 363:446, Roovers et al. (2007) Curr Opin Mol Ther 9:327 and Krah et al. (2016) Immunopharmacol Immunotoxicol 38:21. Single-domain antibodies comprise a single CDR1, a single CDR2 and a single CDR3. Examples of single-domain antibodies are variable fragments of heavy-chain-only antibodies, antibodies that naturally do not comprise light chains, single-domain antibodies derived from conventional antibodies, and engineered antibodies. Single-domain antibodies may be derived from any species including mouse, human, camel, llama, shark, goat, rabbit, and cow. For example, naturally occurring VHH molecules can be derived from antibodies raised in Camelidae species, for example in camel, dromedary, llama, alpaca and guanaco. Like a whole antibody, a single-domain antibody is able to bind selectively to a single specific antigen. Single-domain antibodies may contain only the variable domain of an immunoglobulin chain, i.e., CDR1, CDR2 and CDR3 and framework regions.

“Fab” or “Fab fragment” refers to an antibody fragment composed of VH, CH1, VL, and CL domains.

“F(ab′)₂” or “F(ab′)₂ fragment” refers to an antibody fragment containing two Fab fragments connected by a disulfide bridge in the hinge region.

“Fd” or “Fd fragment” refers to an antibody fragment composed of VH and CH1 domains.

“Fv” or “Fv fragment” refers to an antibody fragment composed of the VH and the VL domains from a single arm of the antibody.

“Host cell” refers to any cell that contains a heterologous nucleic acid. An exemplary heterologous nucleic acid is a vector (e.g., an expression vector).

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks, CDRs, or the constant regions. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” may contain consensus framework sequences derived from human framework sequence analyses, for example as described in Knappik et al., (2000) J Mol Biol 296:57-86, or a synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

“Humanized antibody” refers to an antibody in which at least one CDR is derived from non-human species and at least one framework is derived from human immunoglobulin sequences. Humanized antibody may include substitutions in the frameworks so that the frameworks may not be exact copies of expressed human immunoglobulin or human immunoglobulin germline gene sequences.

“Isolated” refers to a homogenous population of molecules (such as synthetic polynucleotides or polypeptides) which have been substantially separated and/or purified away from other components of the system the molecules are produced in, such as a recombinant cell, as well as a protein that has been subjected to at least one purification or isolation step. “Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“Multispecific” refers to a molecule, such as an antibody that specifically binds two or more distinct antigens or two or more distinct epitopes within the same antigen. Multispecific molecule may have cross-reactivity to other related antigens, for example to the same antigen from other species (homologs), such as human, monkey or ape, for example Macaca fascicularis (cynomolgus, cyno) or Pan troglodytes, or may bind an epitope that is shared between two or more distinct antigens.

“Operatively linked” and similar phrases, when used in reference to nucleic acids or amino acids, refers to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA) and in some instances to the production of a polypeptide (i.e., expression of the open reading frame). Operatively linked peptide refers to a peptide in which the functional domains of the peptide are placed with appropriate distance from each other to impart the intended function of each domain.

“Pharmaceutical composition” refers to a composition that results from combining an active ingredient and a pharmaceutically acceptable carrier.

“Pharmaceutically acceptable carrier” or “excipient” refers to an ingredient in a pharmaceutical composition, other than the active ingredient, which is nontoxic to a subject. Exemplary pharmaceutically acceptable carriers are a buffer, stabilizer or preservative.

“Prevent,” “preventing,” “prevention,” or “prophylaxis” of a disease or disorder means preventing that a disorder occurs in a subject.

The percent “Sequence identity” between two sequences is a function of the number of identical positions shared by the sequences (i.e., % identity=number of identical positions/total number of positions×100), taking into account the number of gaps, and the length of each gap, which need to be introduced for optimal alignment of the two sequences. The percent identity between two amino acid sequences may be determined using the algorithm of E. Meyers and W. Miller (Comput. Appl. Biosci. 4:11-17 (1988)) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch (J. Mol. Biol. 48:444-453 (1970)) algorithm which has been incorporated into the GAP program in the GCG software package (available at www_gcg_com), using either a Blossum 62 matrix or a PAM250 matrix, and a gap weight of 16, 14, 12, 10, 8, 6, or 4 and a length weight of 1, 2, 3, 4, 5, or 6.

“Single chain Fv” or “scFv” refers to a fusion protein comprising at least one antibody fragment comprising a light chain variable region (VL) and at least one antibody fragment comprising a heavy chain variable region (VH), wherein the VL and the VH are contiguously linked via a polypeptide linker, and capable of being expressed as a single chain polypeptide. Unless specified, as used herein, a scFv may have the VL and VH variable regions in either order, e.g., with respect to the N-terminal and C-terminal ends of the polypeptide, the scFv may comprise VL-linker-VH or may comprise VH-linker-VL.

“Specifically binds,” “specific binding,” “specifically binding” or “binds” refer to a proteinaceous molecule binding to, or capable of binding to, an antigen or an epitope within the antigen with greater affinity than for other antigens. Typically, the proteinaceous molecule binds to the antigen or the epitope within the antigen with an equilibrium dissociation constant (K_{D}) of about 1×10⁻⁷ M or less, for example about 5×10⁻⁸ M or less, about 1×10⁻⁸ M or less, about 1×10⁻⁹ M or less, about 1×10⁻¹⁰ M or less, about 1×10⁻¹¹ M or less, or about 1×10⁻¹² M or less, typically with the K_{D} that is at least one hundred-fold less than its K_{D} for binding to a non-specific antigen (e.g., BSA, casein).

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” can be used interchangeably herein.

“T cell” and “T lymphocyte” are interchangeable and used synonymously herein. T cell includes thymocytes, naïve T lymphocytes, memory T cells, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4⁺ T cell) CD4⁺ T cell, a cytotoxic T cell (CTL; CD8⁺ T cell), a tumor infiltrating cytotoxic T cell (TIL; CD8⁺ T cell), CD4⁺CD8⁺ T cell, a gamma-delta T cell, or any other subset of T cells. Also included are “NKT cells”, which refer to a specialized population of T cells that express a semi-invariant αβ T-cell receptor, but also express a variety of molecular markers that are typically associated with NK cells, such as NK1.1. NKT cells include NK1.1⁺ and NK1.1⁻, as well as CD4⁺, CD4⁻, CD8⁺ and CD8⁻ cells. The TCR on NKT cells is unique in that it recognizes glycolipid antigens presented by the MHC I-like molecule CD1d. NKT cells can have either protective or deleterious effects due to their abilities to produce cytokines that promote either inflammation or immune tolerance. Also included are “gamma-delta T cells (γδ T cells),” which refer to specialized populations of T cells possessing a distinct TCR on their surface with an ability to recognize non-classical T cell antigens, and unlike the majority of T cells in which the TCR is composed of two glycoprotein chains designated α- and β-TCR chains, the TCR in γδ T cells is made up of a γ-chain and a δ-chain. Different types of γ-chains and δ-chains exist, such as for example Vγ9 and Vδ2 chains which are co-expressed on Vγ9Vδ2 T cells. γδ T cells can play a role in immunosurveillance and immunoregulation, and were found to be an important source of IL-17 and to induce robust CD8+ cytotoxic T cell responses. Also included are “regulatory T cells” or “Tregs” which refer to T cells that suppress an abnormal or excessive immune response and play a role in immune tolerance. Tregs are typically transcription factor Foxp3-positive CD4+T cells and can also include transcription factor Foxp3-negative regulatory T cells that are IL-10-producing CD4+T cells.

“Therapeutically effective amount” or “effective amount” used interchangeably herein, refers to an amount effective, at dosages and for periods of time necessary, to achieve a desired therapeutic result. A therapeutically effective amount may vary according to factors such as the disease state, age, sex, and weight of the individual, and the ability of a therapeutic or a combination of therapeutics to elicit a desired response in the individual. Example indicators of an effective therapeutic or combination of therapeutics that include, for example, improved wellbeing of the patient, reduction of a tumor burden, arrested or slowed growth of a tumor, and/or absence of metastasis of cancer cells to other locations in the body.

“Treat,” “treating” or “treatment” of a disease or disorder such as cancer refers to accomplishing one or more of the following: reducing the severity and/or duration of the disorder, inhibiting worsening of symptoms characteristic of the disorder being treated, limiting or preventing recurrence of the disorder in subjects that have previously had the disorder, or limiting or preventing recurrence of symptoms in subjects that were previously symptomatic for the disorder.

“Tumor cell” or a “cancer cell” refers to a cancerous, pre-cancerous or transformed cell, either in vivo, ex vivo, or in tissue culture, that has spontaneous or induced phenotypic changes. These changes do not necessarily involve the uptake of new genetic material. Although transformation may arise from infection with a transforming virus and incorporation of new genomic nucleic acid, uptake of exogenous nucleic acid or it can also arise spontaneously or following exposure to a carcinogen, thereby mutating an endogenous gene. Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo.

“Variant,” “mutant” or “altered” refers to a polypeptide or a polynucleotide that differs from a reference polypeptide or a reference polynucleotide by one or more modifications, for example one or more substitutions, insertions or deletions.

The numbering of amino acid residues in the antibody constant region throughout the specification is according to the EU index as described in Kabat et al., Sequences of Proteins of Immunological Interest, 5th Ed. Public Health Service, National Institutes of Health, Bethesda, MD. (1991), unless otherwise explicitly stated.

TABLE 1
Sequence listing:

| | | SEQ ID |
|---|---|---|
| Name | Amino acid sequence | NO: |
| JL5 HCDR1 | NYYWS | 1 |
| JL5 HCDR2 | HIYSTGNIHYNPSLKS | 2 |
| JL5 HCDR3 | DNGAALFDY | 3 |
| JL5 LCDR1 | SGSSSNIGSNIVN | 4 |
| JL5 LCDR2 | SNNQRPS | 5 |
| JL5 LCDR3 | AAWDDSLNGPV | 6 |
| JL6 HCDR1 | SYYWG | 7 |
| JL6 HCDR2 | YIYYSGSTNYNPSLKS | 8 |
| JL6 HCDR3 | MWEILGFDP | 9 |
| JL6 LCDR1 | SGSSSNIGSNPVN | 10 |
| JL6 LCDR2 | SNNQRPS | 11 |
| JL6 LCDR3 | AAWDDSLNGPV | 12 |
| JL2 HCDR1 | SYWMT | 13 |
| JL2 HCDR2 | NIKQDGSERYYVDSVKG | 14 |
| JL2 HCDR3 | EVGYNWNQGGYFDY | 15 |
| JL2 LCDR1 | RSSQSLLHSDGYNYLD | 16 |
| JL2 LCDR2 | LGSYRAS | 17 |
| JL2 LCDR3 | MQVLQTPWT | 18 |
| JL3 HCDR1 | NYYWS | 19 |
| JL3 HCDR2 | HIFSTGHINYDSSLKS | 20 |
| JL3 HCDR3 | DNGAALFDF | 21 |
| JL3 LCDR1 | SGSSSNIGSNIVN | 22 |
| JL3 LCDR2 | SDNQRPS | 23 |
| JL3 LCDR3 | AAWDDSLNGPV | 24 |
| 5D3 CDR1 | NYAMG | 25 |
| 5D3 CDR2 | VISWSGGSTYYADSVKG | 26 |
| 5D3 CDR3 | QFSGASTVVAGTALDYDY | 27 |
| 6H4 CDR1 | NYGMG | 28 |
| 6H4 CDR2 | GISWSGGSTDYADSVKG | 29 |
| 6H4 CDR3 | VFSGAETAYYPSDDYDY | 30 |
| 6H3 CDR1 | NYGMG | 31 |
| 6H3 CDR2 | GITWSGGSTHYADLVKG | 32 |
| 6H3 CDR3 | VFSGAETAYYPSTEYDY | 33 |
| 6G3 CDR1 | NYGMG | 34 |
| 6G3 CDR2 | GISWSGGSTYYADSVKG | 35 |
| 6G3 CDR3 | VFSGAETAQYPSYDYDY | 36 |
| 5C8 CDR1 | NYAMX, wherein X is G or S | 37 |
| 5C8 CDR2 | AISWSGGSTSYADSVKG | 38 |
| 5C8 CDR3 | QFSGADYGFGRLGIRGYEYDY | 39 |
| 5F5 CDR1 | NYAMG | 40 |
| 5F5 CDR2 | AISWSGGSTYYADSVKG | 41 |
| 5F5 CDR3 | MFSGSESQLVVVITNLYEYDY | 42 |
| 6A1 CDR1 | NYAMG | 43 |
| 6A1 CDR2 | TISWSGGSTYYADSVKG | 44 |
| 6A1 CDR3 | AFSGSDYANTKKEVEYDY | 45 |
| 6E4 CDR1 | DYCIA | 46 |
| 6E4 CDR2 | CITTSDGSTYYADSVKG | 47 |
| 6E4 CDR3 | YFGYGCYGGAQDYRAMDY | 48 |
| JL5 VH | QVQLQESGPGLVKPSETLSLTCSVSGASIRNYYWSWIRQTAG | 49 |
| | KGLEWLGHIYSTGNIHYNPSLKSRVTMSVDTSNNQFSLKLRS | |
| | VTAADTAVYYCARDNGAALFDYWGQGTLVTVSS | |
| JL5 VL | QSVLTQPPSASGTPGQRVTISCSGSSSNIGSNIVNWYQQFPGTA | 50 |
| | PKLLIYSNNQRPSGVPDRVSGSKSGTSASLAISGLQSEDEADY | |
| | YCAAWDDSLNGPVFGPGTKVTVL | |
| JL6 VH | QVQLQESGPGLVKPSETLSLTCTVSGGSISSYYWGWIRQPPGK | 51 |
| | GLEWIGYIYYSGSTNYNPSLKSRVTISVDTSKNQFSLKLSSVTA | |
| | ADTAVYYCARMWEILGFDPWGQGTLVTVSS | |
| JL6 VL | QSVLTQPPSASGTPGQRVTISCSGSSSNIGSNPVNWYQQLPGT | 52 |
| | APKLLIYSNNQRPSGVPDRFSGSKSGTSASLAISGLQSEDEADY | |
| | FCAAWDDSLNGPVFGGGTKLTVL | |
| JL2 VH | EVQLVESGGGLVQPGGSLRLSCVVSGFTFSSYWMTWVRQAP | 53 |
| | GKGLEWVANIKQDGSERYYVDSVKGRFTISRDSAKNSLYLQ | |
| | MNSLRAEDTAVYYCAREVGYNWNQGGYFDYWGQGTLVTV | |
| | SS | |
| JL2 VL | DIVMTQSPLSLPVTPGEPASISCRSSQSLLHSDGYNYLDWYLQ | 54 |
| | KSGQSPQLLIYLGSYRASGVPDRFSGSGSGTDFTLKISRVEAED | |
| | VGIYYCMQVLQTPWTFGQGTKVEIK | |
| JL3 VH | QVQLQESGPGLVKPSETLSLTCSVSGGSIRNYYWSWIRQSAGK | 55 |
| | ELEWFGHIFSTGHINYDSSLKSRVTMSVDTSNNQFSLKLRSVT | |
| | AADTAVYYCARDNGAALFDFWGQGTLVTVSS | |
| JL3 VL | QSVLTQPPSASGTPGQRVTISCSGSSSNIGSNIVNWYQQFPGTA | 56 |
| | PKLLLYSDNQRPSGVPDRFSGSKSGTSASLAISGLQSEDEADY | |
| | YCAAWDDSLNGPVFGTGTKVTVL | |
| 5D3 VHH | EVOLVESGGGLVQAGGSLRLSCAASGRPFSNYAMGWFRQ | 57 |
| | APGKEREFVTVISWSGGSTYYADSVKGRFTISRDNAKNTV | |
| | YLQMNSLKPEDTAVYYCAAQFSGASTVVAGTALDYDYW | |
| | GQGTRVTVSS | |
| 6H4 VHH | EVOLVESGGGLVQAGGSLRLSCAASGRPFSNYGMGWFRQ | 58 |
| | APGKKREFVAGISWSGGSTDYADSVKGRFTISRDNAKNTV | |
| | YLQMNSLKPEDTAVYYCAAVFSGAETAYYPSDDYDYWG | |
| | QGTQVTVSS | |
| 6E3 VHH | EVOLVESGGGLVQAGGSLRLSCAASGRPFSNYGMGWFRQ | 59 |
| | APGKKREFVAGISWSGGSTDYADSVKGRLTISRDNAKNTV | |
| | YLQMNSLKPEDTAVYYCAAVFSGAETAYYPSDDYDYWG | |
| | QGTQVTVSS | |
| 6C1 VHH | EVOLVESGGGLVQAGGSLRLSCAASGRPFSNYGMGWFRQ | 60 |
| | APGKKRESVAGISWSGGSTDYADSVKGRFTISRDNAKNTV | |
| | YLQMNSLKPEDTAVYYCAAVFSGAETAYYPSDDYDYWG | |
| | QGTQVTVSS | |
| 6H3 VHH | EVOLVESGGGLVQAGGSLRLSCAVSGRPFSNYGMGWFRQ | 61 |
| | APGKEREFVAGITWSGGSTHYADLVKGRFTISRDNAKNTV | |
| | HLQMNSLKPEDTAVYYCAAVFSGAETAYYPSTEYDYWGQ | |
| | GTQVTVSS | |
| 6G3 VHH | EVOLVESGGGLVQAGGSLRLSCAASGRPENNYGMGWFRQ | 62 |
| | APGKEREFVAGISWSGGSTYYADSVKGRFTISRDNAKNTV | |
| | YLQMNSLKPEDTAVYYCAAVFSGAETAQYPSYDYDYWG | |
| | QGTQVTVSS | |
| 5C8 VHH | EVOLVESGGGLVQAGGSLRLSCAASGRPFSNYAMGWFRQ | 63 |
| | APGKEREFVAAISWSGGSTSYADSVKGRFTISRDNAKNTV | |
| | YLQMNSPKPEDTAIYYCAAQFSGADYGFGRLGIRGYEYDY | |
| | WGQGTQVTVSS | |
| 5F5 VHH | EVOLVESGGGLVQAGGSLRLSCAASGRTFSNYAMGWFRQ | 64 |
| | APGKEREFVAAISWSGGSTYYADSVKGRFTISRDNAKNTV | |
| | YLQMNSLKPEDTAVYYCAAMFSGSESQLVVVITNLYEYD | |
| | YWGQGTQVTVSS | |
| 6A1 VHH | EVOLVESGGGLVQAGGSLRLSCAASGRTFSNYAMGWFRQ | 65 |
| | APGKEREFVATISWSGGSTYYADSVKGRFTISRDNAKNTV | |
| | YLQMNSLKPEDTAVYYCAAAFSGSDYANTKKEVEYDYW | |
| | GQGTQVTVSS | |
| 6E4 VHH | EVOLVESGGGLVQAGGSLRLSCAASGFTFDDYCIAWFRQ | 66 |
| | APGKEREPVSCITTSDGSTYYADSVKGRFTISSDNAKNTV | |
| | YLQMNRLKPEDTAVYYCAAYFGYGCYGGAQDYRAMDY | |
| | WGKGTLVTVSS | |
| Linker 1 | GGSEGKSSGSGSESKSTGGS | 67 |
| Linker 2 | GGGSGGGS | 68 |
| Linker 3 | GGGSGGGSGGGS | 69 |
| Linker 4 | GGGSGGGSGGGSGGGS | 70 |
| Linker 5 | GGGSGGGSGGGSGGGSGGGS | 71 |
| Linker 6 | GGGGSGGGGSGGGGS | 72 |
| Linker 7 | GGGGSGGGGSGGGGSGGGGS | 73 |
| Linker 8 | GGGGSGGGGSGGGGSGGGGSGGGGS | 74 |
| Linker 9 | GSTSGSGKPGSGEGSTKG | 75 |
| Linker 10 | IRPRAIGGSKPRVA | 76 |
| Linker 11 | GKGGSGKGGSGKGGS | 77 |
| Linker 12 | GGKGSGGKGSGGKGS | 78 |
| Linker 13 | GGGKSGGGKSGGGKS | 79 |
| Linker 14 | GKGKSGKGKSGKGKS | 80 |
| Linker 15 | GGGKSGGKGSGKGGS | 81 |
| Linker 16 | GKPGSGKPGSGKPGS | 82 |
| Linker 17 | GKPGSGKPGSGKPGSGKPGS | 83 |
| Linker 18 | GKGKSGKGKSGKGKSGKGKS | 84 |
| Linker 19 | STAGDTHLGGEDFD | 85 |
| Linker 20 | GEGGSGEGGSGEGGS | 86 |
| Linker 21 | GGEGSGGEGSGGEGS | 87 |
| Linker 22 | GEGESGEGESGEGES | 88 |
| Linker 23 | GGGESGGEGSGEGGS | 89 |
| Linker 24 | GEGESGEGESGEGESGEGES | 90 |
| Linker 25 | GSTSGSGKPGSGEGSTKG | 91 |
| Linker 26 | PRGASKSGSASQTGSAPGS | 92 |
| Linker 27 | GTAAAGAGAAGGAAAGAAG | 93 |
| Linker 28 | GTSGSSGSGSGGSGSGGGG | 94 |
| Linker 29 | GKPGSGKPGSGKPGSGKPGS | 95 |
| Linker 30 | GSGS | 96 |
| Linker 31 | APAPAPAPAP | 97 |
| Linker 32 | APAPAPAPAPAPAPAPAPAP | 98 |
| Linker 33 | AEAAAKEAAAKEAAAAKEAAAAKEAAAAKAAA | 99 |
| Linker 34 | GGGGS | 100 |
| JL5x6H4 | QVQLQESGPGLVKPSETLSLTCSVSGASIRNYYWSWIRQTAG | 101 |
| Heavy chain | KGLEWLGHIYSTGNIHYNPSLKSRVTMSVDTSNNQFSLKLRS | |
| 1 | VTAADTAVYYCARDNGAALFDYWGQGTLVTVSSASTKGPSV | |
| | FPLAPSSKSTSGGTAALGCLVKDYFPEPVTVSWNSGALTSGV | |
| | HTFPAVLQSSGLYSLSSVVTVPSSSLGTQTYICNVNHKPSNTK | |
| | VDKKVEPKSCDKTHTCPPCPAPEAAGGPSVFLFPPKPKDTLYI | |
| | TREPEVTCVVVSVSHEDPEVKFNWYVDGVEVHNAKTKPREE | |
| | QYNSTYRVVSVLTVLHQDWLNGKEYKCKVSNKALPAPIEKTI | |
| | SKAKGQPREPQVYTLPPSREEMTKNQVSLSCAVKGFYPSDIA | |
| | VEWESNGQPENNYKTTPPVLDSDGSFFLVSKLTVDKSRWQQ | |
| | GNVFSCSVMHEALHNRFTQKSLSLSPGX | |
| | wherein X is a K that may or may not be | |
| | present | |
| JL5x6H4 | QSVLTQPPSASGTPGQRVTISCSGSSSNIGSNIVNWYQQFPGTA | 102 |
| Light chain 1 | PKLLIYSNNQRPSGVPDRVSGSKSGTSASLAISGLQSEDEADY | |
| | YCAAWDDSLNGPVFGPGTKVTVLGQPKAAPSVTLFPPSSEEL | |
| | QANKATLVCLISDFYPGAVTVAWKADSSPVKAGVETTTPSKQ | |
| | SNNKYAASSYLSLTPEQWKSHRSYSCQVTHEGSTVEKTVAPT | |
| | ECS | |
| JL5x6H4 | EVOLVESGGGLVQAGGSLRLSCAASGRPFSNYGMGWFRQAP | 103 |
| Heavy chain | GKKREFVAGISWSGGSTDYADSVKGRFTISRDNAKNTVYLQ | |
| 2 | MNSLKPEDTAVYYCAAVFSGAETAYYPSDDYDYWGQGTQV | |
| | TVSSEPKSSDKTHTCPPCPAPEAAGGPSVFLFPPKPKDTLYITR | |
| | EPEVTCVVVSVSHEDPEVKFNWYVDGVEVHNAKTKPREEQY | |
| | NSTYRVVSVLTVLHQDWLNGKEYKCKVSNKALPAPIEKTISK | |
| | AKGQPREPQVYTLPPSREEMTKNQVSLWCLVKGFYPSDIAVE | |
| | WESNGQPENNYKTTPPVLDSDGSFFLYSKLTVDKSRWQQGN | |
| | VFSCSVMHEALHNHYTQKSLSLSPGX, | |
| | wherein X is a K that may or may not be | |
| | present | |
| JL5x5D3 | QVQLQESGPGLVKPSETLSLTCSVSGASIRNYYWSWIRQTAG | 104 |
| | KGLEWLGHIYSTGNIHYNPSLKSRVTMSVDTSNNQFSLKLRS | |
| | VTAADTAVYYCARDNGAALFDYWGQGTLVTVSSASTKGPSV | |
| | FPLAPSSKSTSGGTAALGCLVKDYFPEPVTVSWNSGALTSGV | |
| | HTFPAVLQSSGLYSLSSVVTVPSSSLGTQTYICNVNHKPSNTK | |
| | VDKKVEPKSCDKTHTCPPCPAPEAAGGPSVFLFPPKPKDTLYI | |
| | TREPEVTCVVVSVSHEDPEVKFNWYVDGVEVHNAKTKPREE | |
| | QYNSTYRVVSVLTVLHQDWLNGKEYKCKVSNKALPAPIEKTI | |
| | SKAKGQPREPQVYTLPPSREEMTKNQVSLSCAVKGFYPSDIA | |
| | VEWESNGQPENNYKTTPPVLDSDGSFFLVSKLTVDKSRWQQ | |
| | GNVFSCSVMHEALHNRFTQKSLSLSPGX | |
| | wherein X is a K that may or may not be | |
| | present | |
| JL5x5D3 | QSVLTQPPSASGTPGQRVTISCSGSSSNIGSNIVNWYQQFPGTA | 105 |
| | PKLLIYSNNQRPSGVPDRVSGSKSGTSASLAISGLQSEDEADY | |
| | YCAAWDDSLNGPVFGPGTKVTVLGQPKAAPSVTLFPPSSEEL | |
| | QANKATLVCLISDFYPGAVTVAWKADSSPVKAGVETTTPSKQ | |
| | SNNKYAASSYLSLTPEQWKSHRSYSCQVTHEGSTVEKTVAPT | |
| | ECS | |
| JL5x5D3 | EVOLVESGGGLVQAGGSLRLSCAASGRPFSNYAMGWFRQAP | 106 |
| | GKEREFVTVISWSGGSTYYADSVKGRFTISRDNAKNTVYLQM | |
| | NSLKPEDTAVYYCAAQFSGASTVVAGTALDYDYWGQGTRVT | |
| | VSSEPKSSDKTHTCPPCPAPEAAGGPSVFLFPPKPKDTLYITRE | |
| | PEVTCVVVSVSHEDPEVKFNWYVDGVEVHNAKTKPREEQYN | |
| | STYRVVSVLTVLHQDWLNGKEYKCKVSNKALPAPIEKTISKA | |
| | KGQPREPQVYTLPPSREEMTKNQVSLWCLVKGFYPSDIAVEW | |
| | ESNGQPENNYKTTPPVLDSDGSFFLYSKLTVDKSRWQQGNVF | |
| | SCSVMHEALHNHYTQKSLSLSPGX, | |
| | wherein X is a K that may or may not be | |
| | present | |
| JL6x6H4 | QVQLQESGPGLVKPSETLSLTCTVSGGSISSYYWGWIRQPPGK | 107 |
| Heavy chain | GLEWIGYIYYSGSTNYNPSLKSRVTISVDTSKNQFSLKLSSVTA | |
| 1 | ADTAVYYCARMWEILGFDPWGQGTLVTVSSASTKGPSVFPL | |
| | APSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCDKTHTCPPCPAPEAAGGPSVFLFPPKPKDTLYITREP | |
| | EVTCVVVSVSHEDPEVKFNWYVDGVEVHNAKTKPREEQYNS | |
| | TYRVVSVLTVLHQDWLNGKEYKCKVSNKALPAPIEKTISKAK | |
| | GQPREPQVYTLPPSREEMTKNQVSLSCAVKGFYPSDIAVEWE | |
| | SNGQPENNYKTTPPVLDSDGSFFLVSKLTVDKSRWQQGNVFS | |
| | CSVMHEALHNRFTQKSLSLSPGX, | |
| | wherein X is a K that may or may not be | |
| | present | |
| JL6x6H4 | QSVLTQPPSASGTPGQRVTISCSGSSSNIGSNPVNWYQQLPGT | 108 |
| Light chain 1 | APKLLIYSNNQRPSGVPDRFSGSKSGTSASLAISGLQSEDEADY | |
| | FCAAWDDSLNGPVFGGGTKLTVLGQPKAAPSVTLFPPSSEEL | |
| | QANKATLVCLISDFYPGAVTVAWKADSSPVKAGVETTTPSKQ | |
| | SNNKYAASSYLSLTPEQWKSHRSYSCQVTHEGSTVEKTVAPT | |
| | ECS | |
| JL6x6H4 | EVOLVESGGGLVQAGGSLRLSCAASGRPFSNYGMGWFRQAP | 109 |
| Heavy chain | GKKREFVAGISWSGGSTDYADSVKGRFTISRDNAKNTVYLQ | |
| 2 | MNSLKPEDTAVYYCAAVFSGAETAYYPSDDYDYWGQGTQV | |
| | TVSSEPKSSDKTHTCPPCPAPEAAGGPSVFLFPPKPKDTLYITR | |
| | EPEVTCVVVSVSHEDPEVKFNWYVDGVEVHNAKTKPREEQY | |
| | NSTYRVVSVLTVLHQDWLNGKEYKCKVSNKALPAPIEKTISK | |
| | AKGQPREPQVYTLPPSREEMTKNQVSLWCLVKGFYPSDIAVE | |
| | WESNGQPENNYKTTPPVLDSDGSFFLYSKLTVDKSRWQQGN | |
| | VFSCSVMHEALHNHYTQKSLSLSPGX, | |
| | wherein X is a K that may or may not be | |
| | present | |
| JL6x5D3 | QVQLQESGPGLVKPSETLSLTCTVSGGSISSYYWGWIRQPPGK | 110 |
| Heavy chain | GLEWIGYIYYSGSTNYNPSLKSRVTISVDTSKNQFSLKLSSVTA | |
| 1 | ADTAVYYCARMWEILGFDPWGQGTLVTVSSASTKGPSVFPL | |
| | APSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCDKTHTCPPCPAPEAAGGPSVFLFPPKPKDTLYITREP | |
| | EVTCVVVSVSHEDPEVKFNWYVDGVEVHNAKTKPREEQYNS | |
| | TYRVVSVLTVLHQDWLNGKEYKCKVSNKALPAPIEKTISKAK | |
| | GQPREPQVYTLPPSREEMTKNQVSLSCAVKGFYPSDIAVEWE | |
| | SNGQPENNYKTTPPVLDSDGSFFLVSKLTVDKSRWQQGNVFS | |
| | CSVMHEALHNRFTQKSLSLSPGX, | |
| | wherein X is a K that may or may not be | |
| | present | |
| JL6x5D3 | QSVLTQPPSASGTPGQRVTISCSGSSSNIGSNPVNWYQQLPGT | 111 |
| Light chain 1 | APKLLIYSNNQRPSGVPDRFSGSKSGTSASLAISGLQSEDEADY | |
| | FCAAWDDSLNGPVFGGGTKLTVLGQPKAAPSVTLFPPSSEEL | |
| | QANKATLVCLISDFYPGAVTVAWKADSSPVKAGVETTTPSKQ | |
| | SNNKYAASSYLSLTPEQWKSHRSYSCQVTHEGSTVEKTVAPT | |
| | ECS | |
| JL6x5D3 | EVOLVESGGGLVQAGGSLRLSCAASGRPFSNYAMGWFRQAP | 112 |
| Heavy chain | GKEREFVTVISWSGGSTYYADSVKGRFTISRDNAKNTVYLQM | |
| 2 | NSLKPEDTAVYYCAAQFSGASTVVAGTALDYDYWGQGTRVT | |
| | VSSEPKSSDKTHTCPPCPAPEAAGGPSVFLFPPKPKDTLYITRE | |
| | PEVTCVVVSVSHEDPEVKFNWYVDGVEVHNAKTKPREEQYN | |
| | STYRVVSVLTVLHQDWLNGKEYKCKVSNKALPAPIEKTISKA | |
| | KGQPREPQVYTLPPSREEMTKNQVSLWCLVKGFYPSDIAVEW | |
| | ESNGQPENNYKTTPPVLDSDGSFFLYSKLTVDKSRWQQGNVF | |
| | SCSVMHEALHNHYTQKSLSLSPGX, | |
| | wherein X is a K that may or may not be | |
| | present | |
| JL2x6H4 | EVQLVESGGGLVQPGGSLRLSCVVSGFTFSSYWMTWVRQAP | 113 |
| Heavy chain | GKGLEWVANIKQDGSERYYVDSVKGRFTISRDSAKNSLYLQ | |
| 1 | MNSLRAEDTAVYYCAREVGYNWNQGGYFDYWGQGTLVTV | |
| | SSASTKGPSVFPLAPSSKSTSGGTAALGCLVKDYFPEPVTVSW | |
| | NSGALTSGVHTFPAVLQSSGLYSLSSVVTVPSSSLGTQTYICN | |
| | VNHKPSNTKVDKKVEPKSCDKTHTCPPCPAPEAAGGPSVFLF | |
| | PPKPKDTLYITREPEVTCVVVSVSHEDPEVKFNWYVDGVEVH | |
| | NAKTKPREEQYNSTYRVVSVLTVLHQDWLNGKEYKCKVSNK | |
| | ALPAPIEKTISKAKGQPREPQVYTLPPSREEMTKNQVSLSCAV | |
| | KGFYPSDIA VEWESNGQPENNYKTTPPVLDSDGSFFLVSKLTV | |
| | DKSRWQQGNVFSCSVMHEALHNRFTQKSLSLSPGX, | |
| | wherein X is a K that may or may not be | |
| | present | |
| JL2x6H4 | DIVMTQSPLSLPVTPGEPASISCRSSQSLLHSDGYNYLDWYLQ | 114 |
| Light chain 1 | KSGQSPQLLIYLGSYRASGVPDRFSGSGSGTDFTLKISRVEAED | |
| | VGIYYCMQVLQTPWTFGQGTKVEIKRTVAAPSVFIFPPSDEQL | |
| | KSGTASVVCLLNNFYPREAKVQWKVDNALQSGNSQESVTEQ | |
| | DSKDSTYSLSSTLTLSKADYEKHKVYACEVTHQGLSSPVTKSF | |
| | NRGEC | |
| JL2x6H4 | EVOLVESGGGLVQAGGSLRLSCAASGRPFSNYGMGWFRQAP | 115 |
| Heavy chain | GKKREFVAGISWSGGSTDYADSVKGRFTISRDNAKNTVYLQ | |
| 2 | MNSLKPEDTAVYYCAAVFSGAETAYYPSDDYDYWGQGTQV | |
| | TVSSEPKSSDKTHTCPPCPAPEAAGGPSVFLFPPKPKDTLYITR | |
| | EPEVTCVVVSVSHEDPEVKFNWYVDGVEVHNAKTKPREEQY | |
| | NSTYRVVSVLTVLHQDWLNGKEYKCKVSNKALPAPIEKTISK | |
| | AKGQPREPQVYTLPPSREEMTKNQVSLWCLVKGFYPSDIAVE | |
| | WESNGQPENNYKTTPPVLDSDGSFFLYSKLTVDKSRWQQGN | |
| | VFSCSVMHEALHNHYTQKSLSLSPGX, | |
| | wherein X is a K that may or may not be | |
| | present | |
| JL2x5D3 | EVOLVESGGGLVQPGGSLRLSCVVSGFTFSSYWMTWVRQAP | 116 |
| Heavy chain | GKGLEWVANIKQDGSERYYVDSVKGRFTISRDSAKNSLYLQ | |
| 1 | MNSLRAEDTAVYYCAREVGYNWNQGGYFDYWGQGTLVTV | |
| | SSASTKGPSVFPLAPSSKSTSGGTAALGCLVKDYFPEPVTVSW | |
| | NSGALTSGVHTFPAVLQSSGLYSLSSVVTVPSSSLGTQTYICN | |
| | VNHKPSNTKVDKKVEPKSCDKTHTCPPCPAPEAAGGPSVFLF | |
| | PPKPKDTLYITREPEVTCVVVSVSHEDPEVKFNWYVDGVEVH | |
| | NAKTKPREEQYNSTYRVVSVLTVLHQDWLNGKEYKCKVSNK | |
| | ALPAPIEKTISKAKGQPREPQVYTLPPSREEMTKNQVSLSCAV | |
| | KGFYPSDIA VEWESNGQPENNYKTTPPVLDSDGSFFLVSKLTV | |
| | DKSRWQQGNVFSCSVMHEALHNRFTQKSLSLSPGX, | |
| | wherein X is a K that may or may not be | |
| | present | |
| JL2x5D3 | DIVMTQSPLSLPVTPGEPASISCRSSQSLLHSDGYNYLDWYLQ | 117 |
| Light chain 1 | KSGQSPQLLIYLGSYRASGVPDRFSGSGSGTDFTLKISRVEAED | |
| | VGIYYCMQVLQTPWTFGQGTKVEIKRTVAAPSVFIFPPSDEQL | |
| | KSGTASVVCLLNNFYPREAKVQWKVDNALQSGNSQESVTEQ | |
| | DSKDSTYSLSSTLTLSKADYEKHKVYACEVTHQGLSSPVTKSF | |
| | NRGEC | |
| JL2x5D3 | EVOLVESGGGLVQAGGSLRLSCAASGRPFSNYAMGWFRQAP | 118 |
| Heavy chain | GKEREFVTVISWSGGSTYYADSVKGRFTISRDNAKNTVYLQM | |
| 2 | NSLKPEDTAVYYCAAQFSGASTVVAGTALDYDYWGQGTRVT | |
| | VSSEPKSSDKTHTCPPCPAPEAAGGPSVFLFPPKPKDTLYITRE | |
| | PEVTCVVVSVSHEDPEVKFNWYVDGVEVHNAKTKPREEQYN | |
| | STYRVVSVLTVLHQDWLNGKEYKCKVSNKALPAPIEKTISKA | |
| | KGQPREPQVYTLPPSREEMTKNQVSLWCLVKGFYPSDIAVEW | |
| | ESNGQPENNYKTTPPVLDSDGSFFLYSKLTVDKSRWQQGNVF | |
| | SCSVMHEALHNHYTQKSLSLSPGX, | |
| | wherein X is a K that may or may not be | |
| | present | |
| JL3x6H4 | QVQLQESGPGLVKPSETLSLTCSVSGGSIRNYYWSWIRQSAGK | 119 |
| Heavy chain | ELEWFGHIFSTGHINYDSSLKSRVTMSVDTSNNQFSLKLRSVT | |
| 1 | AADTAVYYCARDNGAALFDFWGQGTLVTVSSASTKGPSVFP | |
| | LAPSSKSTSGGTAALGCLVKDYFPEPVTVSWNSGALTSGVHT | |
| | FPAVLQSSGLYSLSSVVTVPSSSLGTQTYICNVNHKPSNTK VD | |
| | KKVEPKSCDKTHTCPPCPAPEAAGGPSVFLFPPKPKDTLYITRE | |
| | PEVTCVVVSVSHEDPEVKFNWYVDGVEVHNAKTKPREEQYN | |
| | STYRVVSVLTVLHQDWLNGKEYKCKVSNKALPAPIEKTISKA | |
| | KGQPREPQVYTLPPSREEMTKNQVSLSCAVKGFYPSDIAVEW | |
| | ESNGQPENNYKTTPPVLDSDGSFFLVSKLTVDKSRWQQGNVF | |
| | SCSVMHEALHNRFTQKSLSLSPGX, | |
| | wherein X is a K that may or may not be | |
| | present | |
| JL3x6H4 | QSVLTQPPSASGTPGQRVTISCSGSSSNIGSNIVNWYQQFPGTA | 120 |
| Light chain 1 | PKLLLYSDNQRPSGVPDRFSGSKSGTSASLAISGLQSEDEADY | |
| | YCAAWDDSLNGPVFGTGTKVTVLGQPKAAPSVTLFPPSSEEL | |
| | QANKATLVCLISDFYPGAVTVAWKADSSPVKAGVETTTPSKQ | |
| | SNNKYAASSYLSLTPEQWKSHRSYSCQVTHEGSTVEKTVAPT | |
| | ECS | |
| JL3x6H4 | EVOLVESGGGLVQAGGSLRLSCAASGRPFSNYGMGWFRQAP | 121 |
| Heavy chain | GKKREFVAGISWSGGSTDYADSVKGRFTISRDNAKNTVYLQ | |
| 2 | MNSLKPEDTAVYYCAAVFSGAETAYYPSDDYDYWGQGTQV | |
| | TVSSEPKSSDKTHTCPPCPAPEAAGGPSVFLFPPKPKDTLYITR | |
| | EPEVTCVVVSVSHEDPEVKFNWYVDGVEVHNAKTKPREEQY | |
| | NSTYRVVSVLTVLHQDWLNGKEYKCKVSNKALPAPIEKTISK | |
| | AKGQPREPQVYTLPPSREEMTKNQVSLWCLVKGFYPSDIAVE | |
| | WESNGQPENNYKTTPPVLDSDGSFFLYSKLTVDKSRWQQGN | |
| | VFSCSVMHEALHNHYTQKSLSLSPGX, | |
| | wherein X is a K that may or may not be | |
| | present | |
| JL3x5D3 | QVQLQESGPGLVKPSETLSLTCSVSGGSIRNYYWSWIRQSAGK | 122 |
| Heavy chain | ELEWFGHIFSTGHINYDSSLKSRVTMSVDTSNNQFSLKLRSVT | |
| 1 | AADTAVYYCARDNGAALFDFWGQGTLVTVSSASTKGPSVFP | |
| | LAPSSKSTSGGTAALGCLVKDYFPEPVTVSWNSGALTSGVHT | |
| | FPAVLQSSGLYSLSSVVTVPSSSLGTQTYICNVNHKPSNTKVD | |
| | KKVEPKSCDKTHTCPPCPAPEAAGGPSVFLFPPKPKDTLYITRE | |
| | PEVTCVVVSVSHEDPEVKFNWYVDGVEVHNAKTKPREEQYN | |
| | STYRVVSVLTVLHQDWLNGKEYKCKVSNKALPAPIEKTISKA | |
| | KGQPREPQVYTLPPSREEMTKNQVSLSCAVKGFYPSDIAVEW | |
| | ESNGQPENNYKTTPPVLDSDGSFFLVSKLTVDKSRWQQGNVF | |
| | SCSVMHEALHNRFTQKSLSLSPGX, | |
| | wherein X is a K that may or may not be | |
| | present | |
| JL3x5D3 | QSVLTQPPSASGTPGQRVTISCSGSSSNIGSNIVNWYQQFPGTA | 123 |
| Light chain 1 | PKLLLYSDNQRPSGVPDRFSGSKSGTSASLAISGLQSEDEADY | |
| | YCAAWDDSLNGPVFGTGTKVTVLGQPKAAPSVTLFPPSSEEL | |
| | QANKATLVCLISDFYPGAVTVAWKADSSPVKAGVETTTPSKQ | |
| | SNNKYAASSYLSLTPEQWKSHRSYSCQVTHEGSTVEKTVAPT | |
| | ECS | |
| JL3x5D3 | EVOLVESGGGLVQAGGSLRLSCAASGRPFSNYAMGWFRQAP | 124 |
| Heavy chain | GKEREFVTVISWSGGSTYYADSVKGRFTISRDNAKNTVYLQM | |
| 2 | NSLKPEDTAVYYCAAQFSGASTVVAGTALDYDYWGQGTRVT | |
| | VSSEPKSSDKTHTCPPCPAPEAAGGPSVFLFPPKPKDTLYITRE | |
| | PEVTCVVVSVSHEDPEVKFNWYVDGVEVHNAKTKPREEQYN | |
| | STYRVVSVLTVLHQDWLNGKEYKCKVSNKALPAPIEKTISKA | |
| | KGQPREPQVYTLPPSREEMTKNQVSLWCLVKGFYPSDIAVEW | |
| | ESNGQPENNYKTTPPVLDSDGSFFLYSKLTVDKSRWQQGNVF | |
| | SCSVMHEALHNHYTQKSLSLSPGX, | |
| | wherein X is a K that may or may not be | |
| | present | |
| IgG1 | ASTKGPSVFPLAPSSKSTSGGTAALGCLVKDYFPEPVTVSWNS | 125 |
| | GALTSGVHTFPAVLQSSGLYSLSSVVTVPSSSLGTQTYICNVN | |
| | HKPSNTKVDKKVEPKSCDKTHTCPPCPAPELLGGPSVFLFPPK | |
| | PKDTLMISRTPEVTCVVVDVSHEDPEVKFNWYVDGVEVHNA | |
| | KTKPREEQYNSTYRVVSVLTVLHQDWLNGKEYKCKVSNKAL | |
| | PAPIEKTISKAKGQPREPQVYTLPPSRDELTKNQVSLTCLVKGF | |
| | YPSDIAVEWESNGQPENNYKTTPPVLDSDGSFFLYSKLTVDKS | |
| | RWQQGNVFSCSVMHEALHNHYTQKSLSLSPGK | |
| IgG2 | ASTKGPSVFPLAPCSRSTSESTAALGCLVKDYFPEPVTVSWNS | 126 |
| | GALTSGVHTFPAVLQSSGLYSLSSVVTVPSSNFGTQTYTCNVD | |
| | HKPSNTKVDKTVERKCCVECPPCPAPPVAGPSVFLFPPKPKDT | |
| | LMISRTPEVTCVVVDVSHEDPEVQFNWYVDGVEVHNAKTKP | |
| | REEQFNSTFRVVSVLTVVHQDWLNGKEYKCKVSNKGLPAPIE | |
| | KTISKTKGQPREPQVYTLPPSREEMTKNQVSLTCLVKGFYPSD | |
| | ISVEWESNGQPENNYKTTPPMLDSDGSFFLYSKLTVDKSRWQ | |
| | QGNVFSCSVMHEALHNHYTQKSLSLSPGK | |
| IgG4 | ASTKGPSVFPLAPCSRSTSESTAALGCLVKDYFPEPVTVSWNS | 127 |
| | GALTSGVHTFPAVLQSSGLYSLSSVVTVPSSSLGTKTYTCNVD | |
| | HKPSNTKVDKRVESKYGPPCPSCPAPEFLGGPSVFLFPPKPKD | |
| | TLMISRTPEVTCVVVDVSQEDPEVQFNWYVDGVEVHNAKTK | |
| | PREEQFNSTYRVVSVLTVLHQDWLNGKEYKCKVSNKGLPSSI | |
| | EKTISKAKGQPREPQVYTLPPSQEEMTKNQVSLTCLVKGFYPS | |
| | DIAVEWESNGQPENNYKTTPPVLDSDGSFFLYSRLTVDKSRW | |
| | QEGNVFSCSVMHEALHNHYTQKSLSLSLGK | |
| Fc region of | EPKSSDKTHTCPPCPAPEAAGGPSVFLFPPKPKDTLMISRTPEV | 128 |
| IgG1 | TCVVVSVSHEDPEVKFNWYVDGVEVHNAKTKPREEQYNSTY | |
| | RVVSVLTVLHQDWLNGKEYKCKVSNKALPAPIEKTISKAKGQ | |
| | PREPQVYTLPPSREEMTKNQVSLWCLVKGFYPSDIAVEWESN | |
| | GQPENNYKTTPPVLDSDGSFFLYSKLTVDKSRWQQGNVFSCS | |
| | VMHEALHNHYTQKSLSLSPGK | |
| human V82 | MQRISSLIHLSLFWAGVMSAIELVPEHQTVPVSIGVPATLRCS | 129 |
| | MKGEAIGNYYINWYRKTQGNTMTFIYREKDIYGPGFKDNFQG | |
| | DIDIAKNLAVLKILAPSERDEGSYYCACDTLGMGGEYTDKLIF | |
| | GKGTRVTVEPRSQPHTKPSVFVMKNGTNVACLVKEFYPKDIR | |
| | INLVSSKKITEFDPAIVISPSGKYNAVKLGKYEDSNSVTCSVQH | |
| | DNKTVHSTDFEVKTDSTDHVKPKETENTKQPSKSCHKPKAIV | |
| | HTEKVNMMSLTVLGLRMLFAKTVAVNFLLTAKLFFL | |
| ScFv CD33 | QSVLTQPPSASGTPGQRVTISCSGSSSNIGSNIVNWYQQFPGTA | 130 |
| JL5 | PKLLIYSNNQRPSGVPDRVSGSKSGTSASLAISGLQSEDEADY | |
| | YCAAWDDSLNGPVFGPGTKVTVL | |
| | GGSEGKSSGSGSESKSTGGS | |
| | QVQLQESGPGLVKPSETLSLTCSVSGASIRNYYWSWIRQTAG | |
| | KGLEWLGHIYSTGNIHYNPSLKSRVTMSVDTSNNQFSLKLRS | |
| | VTAADTAVYYCARDNGAALFDYWGQGTLVTVSS | |
| ScFv CD33 | QSVLTQPPSASGTPGQRVTISCSGSSSNIGSNPVNWYQQLPGT | 131 |
| JL6 | APKLLIYSNNQRPSGVPDRFSGSKSGTSASLAISGLQSEDEADY | |
| | FCAAWDDSLNGPVFGGGTKLTVL | |
| | GGSEGKSSGSGSESKSTGGS | |
| | QVQLQESGPGLVKPSETLSLTCTVSGGSISSYYWGWIRQPPGK | |
| | GLEWIGYIYYSGSTNYNPSLKSRVTISVDTSKNQFSLKLSSVTA | |
| | ADTAVYYCARMWEILGFDPWGQGTLVTVSS | |
| ScFv CD33 | DIVMTQSPLSLPVTPGEPASISCRSSQSLLHSDGYNYLDWYLQ | 132 |
| JL2 | KSGQSPQLLIYLGSYRASGVPDRFSGSGSGTDFTLKISRVEAED | |
| | VGIYYCMQVLQTPWTFGQGTKVEIK | |
| | GGSEGKSSGSGSESKSTGGS | |
| | EVOLVESGGGLVQPGGSLRLSCVVSGFTFSSYWMTWVRQAP | |
| | GKGLEWVANIKQDGSERYYVDSVKGRFTISRDSAKNSLYLQ | |
| | MNSLRAEDTAVYYCAREVGYNWNQGGYFDYWGQGTLVTV | |
| | SS | |
| ScFv CD33 | QSVLTQPPSASGTPGQRVTISCSGSSSNIGSNIVNWYQQFPGTA | 133 |
| JL3 | PKLLLYSDNQRPSGVPDRFSGSKSGTSASLAISGLQSEDEADY | |
| | YCAAWDDSLNGPVFGTGTKVTVL | |
| | GGSEGKSSGSGSESKSTGGS | |
| | QVQLQESGPGLVKPSETLSLTCSVSGGSIRNYYWSWIRQSAGK | |
| | ELEWFGHIFSTGHINYDSSLKSRVTMSVDTSNNQFSLKLRSVT | |
| | AADTAVYYCARDNGAALFDFWGQGTL VTVSS | |

### FURTHER ASPECTS AND EMBODIMENTS OF THE INVENTION

As described above, in a first aspect, the invention relates to an isolated multispecific antibody comprising a first antigen-binding region capable of binding human CD33 and a second antigen-binding region capable of binding a human Vγ9Vδ2 T cell receptor; wherein the first antigen-binding region comprises the HCDR1, the HCDR2, the HCDR3, the LCDR1, the LCDR2 and the LCDR3 of:
a) SEQ ID NOs: 1, 2, 3, 4, 5, and 6, respectively;
a) SEQ ID NOs: 7, 8, 9, 10, 11, and 12, respectively;
d) SEQ ID NOs: 13, 14, 15, 16, 17 and 18, respectively; or
e) SEQ ID NOs: 19, 20, 21, 22, 23 and 24, respectively; and
wherein the second antigen-binding region binds the Vδ2 chain of the Vγ9Vδ2 T cell receptor.

The second antigen-binding region may be a single-domain antibody and comprises the CDR1, CDR2 and CDR3 of:
a) SEQ ID NOs: 25, 26, and 27, respectively;
b) SEQ ID NOs: 28, 29, and 30, respectively;
c) SEQ ID NOs: 31, 32, and 33, respectively;
d) SEQ ID NOs: 34, 35, and 36, respectively
e) SEQ ID NOs: 37, 38, and 39, respectively;
f) SEQ ID NOs: 40, 41, and 42, respectively;
g) SEQ ID NOs: 43, 44, and 45, respectively; or
h) SEQ ID NOs: 46, 47, and 48, respectively.

The first antigen-binding region may comprise the HCDR1, the HCDR2, the HCDR3, the LCDR1, the LCDR2 and the LCDR3 of SEQ ID NOs: 1, 2, 3, 4, 5 and 6, respectively.

The first antigen-binding region may comprise the HCDR1, the HCDR2, the HCDR3, the LCDR1, the LCDR2 and the LCDR3 of SEQ ID NOs: 1, 2, 3, 4, 5 and 6, respectively, and the second antigen-binding region is a single-domain antibody and comprises the CDR1, CDR2 and CDR3 of: SEQ ID NOs: 28, 29, and 30, respectively.

The first antigen-binding region may comprise the HCDR1, the HCDR2, the HCDR3, the LCDR1, the LCDR2 and the LCDR3 of SEQ ID NOs: 1, 2, 3, 4, 5 and 6, respectively, and the second antigen-binding region is a single-domain antibody and comprises the CDR1, CDR2 and CD3 of: SEQ ID NOs: 25, 26, and 27, respectively.

The second antigen-binding region is a single-domain antibody and comprises the CDR1, CDR2 and CD3 of: SEQ ID NOs: 28, 29, and 30, respectively.

The multispecific antibody of the invention may comprise a first antigen-binding region which comprises or consists of:
a) a sequence having at least 90%, 92%, 94%, 96%, 98%, or 100% sequence identity to the VH sequence of SEQ ID NO: 49, and a sequence having at least 90%, 92%, 94%, 96%, 98%, or 100% sequence identity to the VL sequence of SEQ ID NO: 50;
b) a sequence having at least 90%, 92%, 94%, 96%, 98%, or 100% sequence identity to the VH sequence of SEQ ID NO: 51, and a sequence having at least 90%, 92%, 94%, 96%, 98%, or 100% sequence identity to the VL sequence of SEQ ID NO: 52;
c) a sequence having at least 90%, 92%, 94%, 96%, 98%, or 100% sequence identity to the VH sequence of SEQ ID NO: 53, and a sequence having at least 90%, 92%, 94%, 96%, 98%, or 100% sequence identity to the VL sequence of SEQ ID NO: 54;
d) a sequence having at least 90%, 92%, 94%, 96%, 98%, or 100% sequence identity to the VH sequence of SEQ ID NO: 55, and a sequence having at least 90%, 92%, 94%, 96%, 98%, or 100% sequence identity to the VL sequence of SEQ ID NO: 56;
   and
a second antigen-binding region which comprises or consists of:
a) a sequence having at least 90%, 92%, 94%, 96%, 98%, or 100% sequence identity to SEQ ID NO: 57;
b) a sequence having at least 90%, 92%, 94%, 96%, 98%, or 100% sequence identity to SEQ ID NO: 58;
c) a sequence having at least 90%, 92%, 94%, 96%, 98%, or 100% sequence identity to SEQ ID NO: 59;
d) a sequence having at least 90%, 92%, 94%, 96%, 98%, or 100% sequence identity to SEQ ID NO: 60;
e) a sequence having at least 90%, 92%, 94%, 96%, 98%, or 100% sequence identity to SEQ ID NO: 61;
f) a sequence having at least 90%, 92%, 94%, 96%, 98%, or 100% sequence identity to SEQ ID NO: 62;
g) a sequence having at least 90%, 92%, 94%, 96%, 98%, or 100% sequence identity to SEQ ID NO: 63;
h) a sequence having at least 90%, 92%, 94%, 96%, 98%, or 100% sequence identity to SEQ ID NO: 64;
i) a sequence having at least 90%, 92%, 94%, 96%, 98%, or 100% sequence identity to SEQ ID NO: 65; or
j) a sequence having at least 90%, 92%, 94%, 96%, 98%, or 100% sequence identity to SEQ ID NO: 66.

The multispecific antibody of the invention may comprise a second antigen-binding region which competes for binding to human Vδ2 with an antibody having a sequence selected from SEQ ID NO: 57 to 66.

The multispecific antibody of the invention may comprise a second antigen-binding region which binds the same epitope on human Vδ2 as an antibody having a sequence selected from SEQ ID NO: 57 to 66.

The multispecific antibody of the invention may comprise a first antigen-binding region which comprises or consists of:
a) a sequence having at least 90%, 92%, 94%, 96%, 98%, or 100% sequence identity to the VH sequence of SEQ ID NO: 49, and a sequence having at least 90%, 92%, 94%, 96%, 98%, or 100% sequence identity to the VL sequence of SEQ ID NO: 50;
   and
a second antigen-binding region which comprises or consists of:
a) a sequence having at least 90%, 92%, 94%, 96%, 98%, or 100% sequence identity to SEQ ID NO: 57;
b) a sequence having at least 90%, 92%, 94%, 96%, 98%, or 100% sequence identity to SEQ ID NO: 58;
c) a sequence having at least 90%, 92%, 94%, 96%, 98%, or 100% sequence identity to SEQ ID NO: 59;
d) a sequence having at least 90%, 92%, 94%, 96%, 98%, or 100% sequence identity to SEQ ID NO: 60;
e) a sequence having at least 90%, 92%, 94%, 96%, 98%, or 100% sequence identity to SEQ ID NO: 61;
f) a sequence having at least 90%, 92%, 94%, 96%, 98%, or 100% sequence identity to SEQ ID NO: 62;
g) a sequence having at least 90%, 92%, 94%, 96%, 98%, or 100% sequence identity to SEQ ID NO: 63;
h) a sequence having at least 90%, 92%, 94%, 96%, 98%, or 100% sequence identity to SEQ ID NO: 64;
i) a sequence having at least 90%, 92%, 94%, 96%, 98%, or 100% sequence identity to SEQ ID NO: 65; or
j) a sequence having at least 90%, 92%, 94%, 96%, 98%, or 100% sequence identity to SEQ ID NO: 66.

The multispecific antibody of the invention may comprise a first antigen-binding region which comprises or consists of:
b) a sequence having at least 90%, 92%, 94%, 96%, 98%, or 100% sequence identity to the VH sequence of SEQ ID NO: 51, and a sequence having at least 90%, 92%, 94%, 96%, 98%, or 100% sequence identity to the VL sequence of SEQ ID NO: 52;
   and
a second antigen-binding region which comprises or consists of:
a) a sequence having at least 90%, 92%, 94%, 96%, 98%, or 100% sequence identity to SEQ ID NO: 57;
b) a sequence having at least 90%, 92%, 94%, 96%, 98%, or 100% sequence identity to SEQ ID NO: 58;
c) a sequence having at least 90%, 92%, 94%, 96%, 98%, or 100% sequence identity to SEQ ID NO: 59;
d) a sequence having at least 90%, 92%, 94%, 96%, 98%, or 100% sequence identity to SEQ ID NO: 60;
e) a sequence having at least 90%, 92%, 94%, 96%, 98%, or 100% sequence identity to SEQ ID NO: 61;
f) a sequence having at least 90%, 92%, 94%, 96%, 98%, or 100% sequence identity to SEQ ID NO: 62;
g) a sequence having at least 90%, 92%, 94%, 96%, 98%, or 100% sequence identity to SEQ ID NO: 63;
h) a sequence having at least 90%, 92%, 94%, 96%, 98%, or 100% sequence identity to SEQ ID NO: 64;
i) a sequence having at least 90%, 92%, 94%, 96%, 98%, or 100% sequence identity to SEQ ID NO: 65; or
j) a sequence having at least 90%, 92%, 94%, 96%, 98%, or 100% sequence identity to SEQ ID NO: 66.

The multispecific antibody of the invention may comprise a first antigen-binding region which comprises or consists of:
c) a sequence having at least 90%, 92%, 94%, 96%, 98%, or 100% sequence identity to the VH sequence of SEQ ID NO: 53, and a sequence having at least 90%, 92%, 94%, 96%, 98%, or 100% sequence identity to the VL sequence of SEQ ID NO: 54;
   and
a second antigen-binding region which comprises or consists of:
a) a sequence having at least 90%, 92%, 94%, 96%, 98%, or 100% sequence identity to SEQ ID NO: 57;
b) a sequence having at least 90%, 92%, 94%, 96%, 98%, or 100% sequence identity to SEQ ID NO: 58;
c) a sequence having at least 90%, 92%, 94%, 96%, 98%, or 100% sequence identity to SEQ ID NO: 59;
d) a sequence having at least 90%, 92%, 94%, 96%, 98%, or 100% sequence identity to SEQ ID NO: 60;
e) a sequence having at least 90%, 92%, 94%, 96%, 98%, or 100% sequence identity to SEQ ID NO: 61;
f) a sequence having at least 90%, 92%, 94%, 96%, 98%, or 100% sequence identity to SEQ ID NO: 62;
g) a sequence having at least 90%, 92%, 94%, 96%, 98%, or 100% sequence identity to SEQ ID NO: 63;
h) a sequence having at least 90%, 92%, 94%, 96%, 98%, or 100% sequence identity to SEQ ID NO: 64;
i) a sequence having at least 90%, 92%, 94%, 96%, 98%, or 100% sequence identity to SEQ ID NO: 65; or
j) a sequence having at least 90%, 92%, 94%, 96%, 98%, or 100% sequence identity to SEQ ID NO: 66.

The multispecific antibody of the invention may comprise a first antigen-binding region which comprises or consists of:
d) a sequence having at least 90%, 92%, 94%, 96%, 98%, or 100% sequence identity to the VH sequence of SEQ ID NO: 55, and a sequence having at least 90%, 92%, 94%, 96%, 98%, or 100% sequence identity to the VL sequence of SEQ ID NO: 56;
   and
a second antigen-binding region which comprises or consists of:
a) a sequence having at least 90%, 92%, 94%, 96%, 98%, or 100% sequence identity to SEQ ID NO: 57;
b) a sequence having at least 90%, 92%, 94%, 96%, 98%, or 100% sequence identity to SEQ ID NO: 58;
c) a sequence having at least 90%, 92%, 94%, 96%, 98%, or 100% sequence identity to SEQ ID NO: 59;
d) a sequence having at least 90%, 92%, 94%, 96%, 98%, or 100% sequence identity to SEQ ID NO: 60;
e) a sequence having at least 90%, 92%, 94%, 96%, 98%, or 100% sequence identity to SEQ ID NO: 61;
f) a sequence having at least 90%, 92%, 94%, 96%, 98%, or 100% sequence identity to SEQ ID NO: 62;
g) a sequence having at least 90%, 92%, 94%, 96%, 98%, or 100% sequence identity to SEQ ID NO: 63;
h) a sequence having at least 90%, 92%, 94%, 96%, 98%, or 100% sequence identity to SEQ ID NO: 64;
i) a sequence having at least 90%, 92%, 94%, 96%, 98%, or 100% sequence identity to SEQ ID NO: 65; or
j) a sequence having at least 90%, 92%, 94%, 96%, 98%, or 100% sequence identity to SEQ ID NO: 66.

The multispecific antibody of the invention may comprise a first antigen-binding region which comprises or consists of:
a) a sequence having at least 90%, 92%, 94%, 96%, 98%, or 100% sequence identity to the VH sequence of SEQ ID NO: 49, and a sequence having at least 90%, 92%, 94%, 96%, 98%, or 100% sequence identity to the VL sequence of SEQ ID NO: 50;
b) a sequence having at least 90%, 92%, 94%, 96%, 98%, or 100% sequence identity to the VH sequence of SEQ ID NO: 51, and a sequence having at least 90%, 92%, 94%, 96%, 98%, or 100% sequence identity to the VL sequence of SEQ ID NO: 52;
c) a sequence having at least 90%, 92%, 94%, 96%, 98%, or 100% sequence identity to the VH sequence of SEQ ID NO: 53, and a sequence having at least 90%, 92%, 94%, 96%, 98%, or 100% sequence identity to the VL sequence of SEQ ID NO: 54;
d) a sequence having at least 90%, 92%, 94%, 96%, 98%, or 100% sequence identity to the VH sequence of SEQ ID NO: 55, and a sequence having at least 90%, 92%, 94%, 96%, 98%, or 100% sequence identity to the VL sequence of SEQ ID NO: 56;
   and
a second antigen-binding region which comprises or consists of:
b) a sequence having at least 90%, 92%, 94%, 96%, 98%, or 100% sequence identity to SEQ ID NO: 58.

The multispecific antibody of the invention may comprise a first antigen-binding region which comprises or consists of:
a) a sequence having at least 90%, 92%, 94%, 96%, 98%, or 100% sequence identity to the VH sequence of SEQ ID NO: 49, and a sequence having at least 90%, 92%, 94%, 96%, 98%, or 100% sequence identity to the VL sequence of SEQ ID NO: 50;
b) a sequence having at least 90%, 92%, 94%, 96%, 98%, or 100% sequence identity to the VH sequence of SEQ ID NO: 51, and a sequence having at least 90%, 92%, 94%, 96%, 98%, or 100% sequence identity to the VL sequence of SEQ ID NO: 52;
c) a sequence having at least 90%, 92%, 94%, 96%, 98%, or 100% sequence identity to the VH sequence of SEQ ID NO: 53, and a sequence having at least 90%, 92%, 94%, 96%, 98%, or 100% sequence identity to the VL sequence of SEQ ID NO: 54;
d) a sequence having at least 90%, 92%, 94%, 96%, 98%, or 100% sequence identity to the VH sequence of SEQ ID NO: 55, and a sequence having at least 90%, 92%, 94%, 96%, 98%, or 100% sequence identity to the VL sequence of SEQ ID NO: 56;
   and
a second antigen-binding region which comprises or consists of:
a) a sequence having at least 90%, 92%, 94%, 96%, 98%, or 100% sequence identity to SEQ ID NO: 57.

Variants of the sequences disclosed herein preferably comprise conservative modifications of the disclosed sequence. “Conservative modifications” refer to amino acid modifications that do not significantly affect or alter the binding characteristics of the antibody containing the amino acid modifications. Conservative modifications include amino acid substitutions, additions and deletions. Conservative amino acid substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis (MacLennan et al., (1988) Acta Physiol Scand Suppl 643:55-67; Sasaki et al., (1988) Adv Biophys 35:1-24). Amino acid substitutions to the antibodies of the invention may be made by known methods for example by PCR mutagenesis (U.S. Pat. No. 4,683,195). Alternatively, libraries of variants may be generated for example using random (NNK) or non-random codons, for example DVK codons, which encode 11 amino acids (Ala, Cys, Asp, Glu, Gly, Lys, Asn, Arg, Ser, Tyr, Trp). The resulting variants may be tested for their characteristics using assays described herein.

The multispecific antibody of the invention may have any suitable antibody format. Many antibody formats have been described in the art.

The multispecific antibody may comprise a Fab, an scFv, a (scFv)₂, a Fv, a F(ab′)₂ or a Fd comprising the first antigen-binding region capable of binding human CD33.

The second antigen-binding region capable of binding a human Vγ9Vδ2 T cell receptor may be of any suitable format, e.g., a Fab, an scFv, a (scFv)₂, a Fv, a F(ab′)₂, a Fd or a single-domain antibody. The multispecific antibody may comprise a single-domain antibody comprising or consisting of the second antigen-binding region capable of binding a human Vγ9Vδ2 T cell receptor.

The multispecific antibody may comprise a Fab comprising the first antigen-binding region capable of binding human CD33, and a single-domain antibody comprising the second antigen-binding region capable of binding a human Vγ9Vδ2 T cell receptor.

The multispecific antibody may comprise an scFv comprising the first antigen-binding region capable of binding human CD33, and a single-domain antibody comprising the second antigen-binding region capable of binding a human Vγ9Vδ2 T cell receptor.

Depending on the antibody format, the antigen-binding regions or parts thereof may be part of the same polypeptide chain and be expressed from a single open reading frame. In such embodiments, linker sequences may be used between the antigen-binding region sequences.

The multispecific antibody may comprise an scFv comprising the first antigen-binding region capable of binding human CD33, and a VHH comprising the second antigen-binding region capable of binding a human Vγ9Vδ2 T cell receptor, and the scFv comprises a peptide linker, optionally selected from the group of linkers set forth in SEQ ID NO:67 to 99, such as SEQ ID NO: 67.

The first antigen-binding region and second antigen-binding region may be directly or indirectly covalently linked via a peptide linker, optionally wherein the peptide linker comprises or consists of the sequence set forth in SEQ ID NO: 100.

The multispecific antibody may be encoded by a single open reading frame wherein the first antigen-binding region is located N-terminally of the second antigen-binding region. The order from N-terminus to C-terminus may be VL-first linker-VH-second linker-VHH, wherein VL is the light chain variable region of the first antigen-binding domain, first linker is a peptide linker, VH is the heavy chain variable region of the first antigen-binding domain, second linker is a peptide linker and VHH is a single-domain antibody comprising the second antigen-binding region. The multispecific antibody may be encoded by a single open reading frame wherein the first antigen-binding region is located C-terminally of the second antigen-binding region.

The multispecific antibody of the invention may comprise constant region sequences, such as an Fc region consisting of a first Fc polypeptide and a second Fc polypeptide. Thus, the multispecific antibody may comprise a Fab comprising the first antigen-binding region capable of binding human CD33, a VHH comprising the second antigen-binding region capable of binding a human Vγ9Vδ2 T cell receptor, and an Fc region.

The multispecific antibody may comprise:
a first polypeptide comprising the VH of SEQ ID NO: 49 and a heavy chain constant sequence,
a second polypeptide comprising the VL of SEQ ID NO: 50 and a light chain constant sequence, and
a third polypeptide comprising the VHH of SEQ ID NO: 58 and an Fc sequence.

The multispecific antibody may comprise:
a first polypeptide comprising the VH of SEQ ID NO: 49 and a heavy chain constant sequence,
a second polypeptide comprising the VL of SEQ ID NO: 50 and a light chain constant sequence, and
a third polypeptide comprising the VHH of SEQ ID NO: 57 and an Fc sequence.

The multispecific antibody may comprise:
a first polypeptide comprising the VH of SEQ ID NO: 51 and a heavy chain constant sequence,
a second polypeptide comprising the VL of SEQ ID NO: 52 and a light chain constant sequence, and
a third polypeptide comprising the VHH of SEQ ID NO: 58 and an Fc sequence.

The multispecific antibody may comprise:
a first polypeptide comprising the VH of SEQ ID NO: 51 and a heavy chain constant sequence,
a second polypeptide comprising the VL of SEQ ID NO: 52 and a light chain constant sequence, and
a third polypeptide comprising the VHH of SEQ ID NO: 57 and an Fc sequence.

The multispecific antibody may comprise:
a first polypeptide comprising the VH of SEQ ID NO: 53 and a heavy chain constant sequence,
a second polypeptide comprising the VL of SEQ ID NO: 54 and a light chain constant sequence, and a third polypeptide comprising the VHH of SEQ ID NO: 58 and an Fc sequence.

The multispecific antibody may comprise:
a first polypeptide comprising the VH of SEQ ID NO: 53 and a heavy chain constant sequence,
a second polypeptide comprising the VL of SEQ ID NO: 54 and a light chain constant sequence, and
a third polypeptide comprising the VHH of SEQ ID NO: 57 and an Fc sequence.

The multispecific antibody may comprise:
a first polypeptide comprising the VH of SEQ ID NO: 55 and a heavy chain constant sequence,
a second polypeptide comprising the VL of SEQ ID NO: 56 and a light chain constant sequence, and
a third polypeptide comprising the VHH of SEQ ID NO: 58 and an Fc sequence.

The multispecific antibody may comprise:
a first polypeptide comprising the VH of SEQ ID NO: 55 and a heavy chain constant sequence,
a second polypeptide comprising the VL of SEQ ID NO: 56 and a light chain constant sequence, and
a third polypeptide comprising the VHH of SEQ ID NO: 57 and an Fc sequence.

The isolated multispecific antibody of the invention may comprise or consist of:
a) the polypeptides set forth in SEQ ID NO: 101, 102 and 103;
b) the polypeptides set forth in SEQ ID NO:104, 105 and 106;
c) the polypeptides set forth in SEQ ID NO: 107, 108 and 109;
d) the polypeptides set forth in SEQ ID NO:110, 111 and 112;
e) the polypeptides set forth in SEQ ID NO:113, 114 and 115;
f) the polypeptides set forth in SEQ ID NO:116, 117 and 118;
g) the polypeptides set forth in SEQ ID NO:119, 120 and 121; or
h) the polypeptides set forth in SEQ ID NO: 122, 123 and 124.

The isolated multispecific antibody of the invention may be a bispecific antibody.

The isolated multispecific antibody of the invention may bind monovalently to CD33 and bind monovalently to the human Vγ9Vδ2 T cell receptor.

The first antigen-binding region and/or second antigen-binding region of the antibody of the invention may be human or humanized.

The multispecific antibody may be able to induce proliferation of human Vγ9Vδ2 T cells in the presence of target cells with a more than 10-fold or more than 50-fold increase after 10 days at an antibody concentration of 1 nM, e.g., when tested as described in the Examples herein.

The multispecific antibody of the invention may be capable of mediating killing of THP-1 cells in the presence of Vγ9Vδ2-T cells, including at low effector to target cell ratios. Thus, the multispecific antibody of the invention may be capable of mediating killing of THP-1 cells (T) in the presence of Vγ9Vδ2-T cells (E) with an EC50 of below 1 nM, such as 0.5 nM, such as below 200 pM, such as below 150 pM, such as below 100 pM at an E:T ratio of 1:20, e.g., when tested as described in the Examples herein.

The multispecific antibody of the invention may be capable of mediating killing of human CD33-expressing cells from a hematologic cancer patient.

The multispecific antibody of the invention may be able to preferentially mediate killing of CD33-positive tumor cells, e.g., THP-1 cells, over non-tumor cells, e.g. CD14+ cells from a healthy donor, e.g., when tested as described in the Examples herein.

### Isotypes, Allotypes and Fc Engineering

The Ig constant region or the fragment of the Ig constant region, such as the Fc region present in the antibodies of the disclosure may be of any allotype or isotype.

The isolated multispecific antibody of the present disclosure may comprise an Ig constant region or a fragment thereof, e.g., a fragment crystallizable region (“Fc” region). Said Ig constant region or fragment thereof is selected from the group consisting of an IgG1, an IgG2, an IgG3, or an IgG4 isotype.

The Ig constant region or the fragment of the Ig constant region may be of any allotype. It is expected that the allotype has no influence on properties of the Ig constant region, such as binding or Fc-mediated effector functions. Immunogenicity of therapeutic antibodies comprising Ig constant regions of fragments thereof is associated with increased risk of infusion reactions and decreased duration of therapeutic response (Baert et al., (2003) N Engl J Med 348:602-08). The extent to which therapeutic antibodies comprising Ig constant regions of fragments thereof induce an immune response in the host may be determined in part by the allotype of the Ig constant region (Stickler et al., (2011) Genes and Immunity 12:213-21). Ig constant region allotype is related to amino acid sequence variations at specific locations in the constant region sequences of the antibody. Table 2 shows select IgG1, IgG2 and IgG4 allotypes.

TABLE 2

| | Amino acid residue at position of diversity | | | | | | | |
|---|---|---|---|---|---|---|---|---|
| | (residue numbering: EU Index) | | | | | | | |
| | IgG2 | | IgG4 | | IgG1 | | | |
| Allotype | 189 | 282 | 309 | 422 | 214 | 356 | 358 | 431 |
| G2m(n) | T | M | | | | | | |
| G2m(n−) | P | V | | | | | | |
| G2m(n)/(n−) | T | V | | | | | | |
| nG4m(a) | | | L | R | | | | |
| G1m(17) | | | | | K | E | M | A |
| G1m(17, 1) | | | | | K | D | L | A |
| G1m(3) | | | | | R | E | M | A |

C-terminal lysine (CTL) may be removed from the Ig constant region by endogenous circulating carboxypeptidases in the blood stream (Cai et al., (2011) Biotechnol Bioeng 108:404-412). During manufacturing, CTL removal may be controlled to less than the maximum level by control of concentration of extracellular Zn²⁺, EDTA or EDTA−Fe³⁺ as described in U.S. Patent Publ. No. US20140273092. CTL content of proteins may be measured using known methods.

The Ig constant region conjugated to the antigen-binding fragments may have a C-terminal lysine content from 0% to 100%, from about 10% to about 90%, from about 20% to about 80%, from about 40% to about 70%, from about 55% to about 70%, or about 60%.

Fc region mutations may be made to the Ig constant region or a fragment thereof conjugated to the antigen binding domains, to modulate their binding to Fc receptors and thereby their effector functions such as ADCC, ADCP and/or ADCP and/or pharmacokinetic properties. This may be achieved by introducing mutation(s) into the Fc that (i) modulate binding of the mutated Fc to activating FcγRs (FcγRI, FcγRIIa, FcγRIII), and inhibitory FcγRIIb; and/or (ii) reduce Fc effector functions such as C1q binding, complement dependent cytotoxicity (CDC), antibody-dependent cell-mediated cytotoxicity (ADCC) or phagocytosis (ADCP), reduce antigen-independent T cell activation mediated through binding of the bispecific antibody to Fc receptor-positive cells.

The Fc region of the isolated multispecific antibody of the disclosure may be inert. The inert Fc region of the isolated multispecific antibody of the disclosure may comprise in one or both of the first and second Fc polypeptides, an Ala at a position corresponding to 234, an Ala at a position corresponding to 235, and a Ser at a position corresponding to 265, wherein the numbering is according to Eu.

The Fc region may comprise at least one mutation that results in reduced binding of the antibody to a Fcγ receptor (FcγR). Fc positions that may be mutated to reduce binding of the antibody to the activating FcγR and subsequently to reduce effector function include positions 214, 233, 234, 235, 236, 237, 238, 265, 267, 268, 270, 295, 297, 309, 327, 328, 329, 330, 331 and 365. Exemplary mutations that may be made singularly or in combination are mutations K214T, E233P, L234V, L234A, deletion of G236, V234A, F234A, L235A, G237A, P238A, P238S, D265A, S267E, H268A, H268Q, Q268A, N297A, A327Q, P329A, D270A, Q295A, V309L, A327S, L328F, A330S and P331S in IgG1, IgG2, IgG3 or IgG4. Exemplary combination mutations that result in antibodies with reduced ADCC are mutations L234A/L235A on IgG1, L234A/L235A/D265S on IgG1, V234A/G237A/P238S/H268A/V309L/A330S/P331S on IgG2, F234A/L235A on IgG4, S228P/F234A/L235A on IgG4, N297A on all Ig isotypes, V234A/G237A on IgG2, K214T/E233P/L234V/L235A/G236-deleted/A327G/P331A/D365E/L358M on IgG1, H268Q/V309L/A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4, and S228P/F234A/L235A/G236-deleted/G237A/P238S on IgG4. Hybrid IgG2/4 Fc domains may also be used, such as Fc with residues 117-260 from IgG2 and residues 261-447 from IgG4.

An exemplary mutation that results in antibodies with reduced CDC is a K322A mutation.

Well-known S228P mutation may be made in IgG4 to enhance IgG4 stability.

The at least one mutation that results in reduced binding of the antibody to the FcγR may be selected from the group consisting of F234A/L235A, L234A/L235A, L234A/L235A/D265S, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index.

The antibody of the disclosure may comprise at least one mutation in the Fc region that enhances binding of the protein to an Fcγ receptor (FcγR) and/or enhances Fc effector functions such as C1q binding, complement dependent cytotoxicity (CDC), antibody-dependent cell-mediated cytotoxicity (ADCC) and/or phagocytosis (ADCP).

The Fc region may comprise at least one mutation that results in enhanced binding of the antibody to the FcγR. The at least one mutation that results in enhanced binding of the protein to the FcγR is selected from the group consisting of S239D/1332E, S298A/E333A/K334A, F243L/R292P/Y300L, F243L/R292P/Y300L/P396L, F243L/R292P/Y300L/V305I/P396L and G236A/S239D/1332E, wherein residue numbering is according to the EU index.

Fc positions that may be mutated to increase binding of the antibody to the activating FcγR and/or enhance Fc effector functions include positions 236, 239, 243, 256, 290, 292, 298, 300, 305, 312, 326, 330, 332, 333, 334, 345, 360, 339, 378, 396 or 430 (residue numbering according to the EU index). Exemplary mutations that may be made singularly or in combination are G236A, S239D, F243L, T256A, K290A, R292P, S298A, Y300L, V305L, K326A, A330K, 1332E, E333A, K334A, A339T and P396L. Exemplary combination mutations that result in antibodies with increased ADCC or ADCP are a S239D/1332E, S298A/E333A/K334A, F243L/R292P/Y300L, F243L/R292P/Y300L/P396L, F243L/R292P/Y300L/V305I/P396L and G236A/S239D/1332E.

Fc positions that may be mutated to enhance CDC include positions 267, 268, 324, 326, 333, 345 and 430. Exemplary mutations that may be made singularly or in combination are S267E, F1268F, S324T, K326A, K326W, E333A, E345K, E345Q, E345R, E345Y, E430S, E430F and E430T. Exemplary combination mutations that result in antibodies with increased CDC are K326A/E333A, K326W/E333A, H268F/S324T, S267E/H268F, S267E/S324T and S267E/H268F/S324T.

The specific mutations described herein are mutations when compared to the IgG1, IgG2 and IgG4 wild-type amino acid sequences of SEQ ID NOs: 125, 126 and 127, respectively.

Binding of the antibody to FcγR or FcRn may be assessed on cells engineered to express each receptor using flow cytometry. In an exemplary binding assay, 2×10⁵ cells per well are seeded in 96-well plate and blocked in BSA Stain Buffer (BD Biosciences, San Jose, USA) for 30 min at 4° C. Cells are incubated with a test antibody on ice for 1.5 hour at 4° C. After being washed twice with BSA stain buffer, the cells are incubated with R-PE labeled anti-human IgG secondary antibody (Jackson Immunoresearch Laboratories) for 45 min at 4° C.

The cells are washed twice in stain buffer and then resuspended in 150 μL of Stain Buffer containing 1:200 diluted DRAQ7 live/dead stain (Cell Signaling Technology, Danvers, USA). PE and DRAQ7 signals of the stained cells are detected by Miltenyi MACSQuant flow cytometer (Miltenyi Biotec, Auburn, USA) using B2 and B4 channel respectively. Live cells are gated on DRAQ7 exclusion and the geometric mean fluorescence signals are determined for at least 10,000 live events collected. FlowJo software (Tree Star) is used for analysis. Data is plotted as the logarithm of antibody concentration versus mean fluorescence signals. Nonlinear regression analysis is performed.

Fc positions that may be mutated to modulate half-life (e.g., binding to FcRn) include positions 250, 252, 253, 254, 256, 257, 307, 376, 380, 428, 434 and 435. Exemplary mutations that may be made singularly or in combination are mutations T250Q, M252Y, I253A, S254T, T256E, P257I, T307A, D376V, E380A, M428L, H433K, N434S, N434A, N434H, N434F, H435A and H435R. Exemplary singular or combination mutations that may be made to increase the half-life are mutations M428L/N434S, M252Y/S254T/T256E, T250Q/M428L, N434A and T307A/E380A/N434A. Exemplary singular or combination mutations that may be made to reduce the half-life are mutations H435A, P257I/N434H, D376V/N434H, M252Y/S254T/T256E/H433K/N434F, T308P/N434A and H435R.

In other words, in the isolated multispecific antibody of the disclosure, the Fc region, in one or both of the first and second Fc polypeptides, may comprise a Tyr at a position corresponding to 252, a Thr at a position corresponding to 254, and a Glu at a position corresponding to 256, wherein the numbering is according to Eu.

The antigen binding fragments of the disclosure may be engineered into full length multispecific antibodies which may be generated using Fab arm exchange, in which substitutions are introduced into two monospecific bivalent antibodies within the Ig constant region CH3 domain which promote Fab arm exchange in vitro. In the methods, two monospecific bivalent antibodies may be engineered to have certain substitutions at the CH3 domain that promote heterodimer stability; the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl) phosphine (TCEP), L-cysteine and beta-mercaptoethanol, preferably a reducing agent selected from the group consisting of: 2-mercaptoethylamine, dithiothreitol and tris(2-carboxyethyl) phosphine. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

CH3 mutations that may be used include technologies such as Knob-in-Hole mutations (Genentech), electrostatically-matched mutations (Chugai, Amgen, NovoNordisk, Oncomed, Merus), the Strand Exchange Engineered Domain body (SEEDbody) (EMD Serono), Duobody® mutations (Genmab), and other asymmetric mutations (e.g., Zymeworks).

Knob-in-hole mutations are disclosed for example in WO1996/027011 and include mutations on the interface of CH3 region in which an amino acid with a small side chain (hole) is introduced into the first CH3 region and an amino acid with a large side chain (knob) is introduced into the second CH3 region, resulting in preferential interaction between the first CH3 region and the second CH3 region. Exemplary CH3 region mutations forming a knob and a hole are T366Y/F405A, T366W/F405W, F405W/Y407A, T394W/Y407T, T394S/Y407A, T366W/T394S, F405W/T394S, and T366W/T366S_L368A_Y407V. In other words, in the isolated multispecific antibody of the disclosure, the first Fc polypeptide may comprise a Trp at a position corresponding to 366, and the second Fc polypeptide may comprise a Ser at a position corresponding to 366, an Ala at a position corresponding to 368 and a Val at a position corresponding to 407, or vice versa, and wherein the numbering is according to Eu.

Heavy chain heterodimer formation may be promoted by using electrostatic interactions by substituting positively charged residues on the first CH3 region and negatively charged residues on the second CH3 region as described in US2010/0015133, US2009/0182127, US2010/028637 or US2011/0123532.

Other asymmetric mutations that can be used to promote heavy chain heterodimerization are L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in US2012/0149876 or US2013/0195849 (Zymeworks).

SEEDbody mutations involve substituting select IgG residues with IgA residues to promote heavy chain heterodimerization as described in US20070287170.

Other exemplary mutations that may be used are R409D_K370E/D399K_E357K, S354C_T366W/Y349C_T366S_L368A_Y407V, Y349C_T366W/S354C_T366S_L368A_Y407V, T366K/L351D, L351K/Y349E, L351K/Y349D, L351K/L368E, L351Y_Y407A/T366A_K409F, L351Y_Y407A/T366V_K409F, K392D/D399K, K392D/E356K, K253E D282K_K322D/D239K_E240K_K292D, K392D_K409D/D356K_D399K as described in WO2007/147901, WO 2011/143545, WO2013157954, WO2013096291 and US2018/0118849.

Duobody® mutations (Genmab) are disclosed for example in U.S. Pat. No. 9,150,663 and US2014/0303356 and include mutations F405L/K409R, wild-type/F405L_R409K, T350I_K370T_F405L/K409R, K370W/K409R, D399AFGHILMNRSTVWY/K409R, T366ADEFGHILMQVY/K409R, L368ADEGHNRSTVQ/K409AGRH, D399FHKRQ/K409AGRH, F405IKLSTVW/K409AGRH and Y407LWQ/K409AGRH.

Additional bispecific or multispecific structures include Dual Variable Domain Immunoglobulins (DVD) (Int. Pat. Publ. No. WO2009/134776; DVDs are full length antibodies comprising the heavy chain having a structure VH1-linker-VH2-CH and the light chain having the structure VL1-linker-VL2-CL; linker being optional), structures that include various dimerization domains to connect the two antibody arms with different specificity, such as leucine zipper or collagen dimerization domains (Int. Pat. Publ. No. WO2012/022811, U.S. Pat. Nos. 5,932,448; 6,833,441), two or more domain antibodies (dAbs) conjugated together, diabodies, heavy chain only antibodies such as camelid antibodies and engineered camelid antibodies, Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer), IgG-like Bispecific (InnClone/Eli Lilly), Ts2Ab (MedImmune/AZ) and BsAb (Zymogenetics), HERCULES (Biogen Idec) and TvAb (Roche), ScFv/Fc Fusions (Academic Institution), SCORPION (Emergent BioSolutions/Trubion, Zymogenetics/BMS), Dual Affinity Retargeting Technology (Fc-DART) (MacroGenics) and Dual (ScFv) 2-Fab (National Research Center for Antibody Medicine-China), Dual-Action or Bis-Fab (Genentech), Dock-and-Lock (DNL) (ImmunoMedics), Bivalent Bispecific (Biotecnoland Fab-Fv (UCB-Celltech). ScFv-, diabody-based, and domain antibodies, include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

In the isolated multispecific antibody of the disclosure, the first Ig constant region or the fragment of the first Ig constant region and the second Ig constant region or the fragment of the second Ig constant region may comprise at least one mutation that modulates binding to protein A. Such modifications may be advantageous for purification purposes during antibody production. Such at least one mutation may be present in the first or the second Fc polypeptides, or in both.

The at least one mutation that modulates binding to protein A is H435R/Y436F, wherein residue numbering is according to the EU index. The first Ig constant region or the fragment of the first Ig constant region and the second Ig constant region or the fragment of the second Ig constant region may comprise the L234A/L235A/D265S, the M252Y/S254T/T256E and the H435R/Y436F mutations, wherein residue numbering is according to the EU index.

The antigen binding domains of the disclosure may also be engineered into multispecific antibodies which comprise three polypeptide chains. In such designs, at least one antigen binding domain is in the form of a scFv. Exemplary designs include (in which “1” indicates the first antigen binding domain, “2” indicates the second antigen binding domain and “3” indicates the third antigen binding domain:
Design 1: Chain A) scFv1-CH2-CH3; Chain B) VL2-CL; Chain C) VH2-CH1-hinge-CH2-CH3
Design 2: Chain A) scFv1-hinge-CH2-CH3; Chain B) VL2-CL; Chain C) VH2-CH1-hinge-CH2-CH3
Design 3: Chain A) scFv1-CH1-hinge-CH2-CH3; Chain B) VL2-CL; Chain C) VH2-CH1-hinge-CH2-CH3 Design 4: Chain A) CH2-CH3-scFv1; Chain B) VL2-CL; Chain C) VH2-CH1-hinge-CH2-CH3

CH3 engineering may be incorporated to the Designs 1-4, such as mutations L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in US2012/0149876 or US2013/0195849 (Zymeworks).

### Glycoengineering

The ability of the antigen binding domains conjugated to the Ig constant regions or to the fragments of the Ig constant regions to mediate ADCC can be enhanced by engineering the Ig constant regions or the fragments of the Ig constant regions oligosaccharide component. Human IgG1 or IgG3 may be N-glycosylated at Asn297 with the majority of the glycans in the well-known biantennary G0, G0F, G1, G1F, G2 or G2F forms. Ig constant region containing antibodies produced by non-engineered CHO cells typically have a glycan fucose content of about at least 85%. The removal of the core fucose from the biantennary complex-type oligosaccharides attached to the antigen binding domains conjugated to the Ig constant regions or to the fragments of the Ig constant region enhances the ADCC of the antibody via improved FcγRIIIa binding without altering antigen binding or CDC activity. Such antibodies can be achieved using different methods reported to lead to the successful expression of relatively high defucosylated immunoglobulins bearing the biantennary complex-type of Fc oligosaccharides such as control of culture osmolality (Konno et al., Cytotechnology 64 (: 249-65, 2012), application of a variant CHO line Lec13 as the host cell line (Shields et al., J Biol Chem 277:26733-26740, 2002), application of a variant CHO line EB66 as the host cell line (Olivier et al., MAbs; 2 (4): 405-415, 2010; PMID: 20562582), application of a rat hybridoma cell line YB2/0 as the host cell line (Shinkawa et al., J Biol Chem 278:3466-3473, 2003), introduction of small interfering RNA specifically against the a 1,6-fucosyltrasferase (FUT8) gene (Mori et al., Biotechnol Bioeng 88:901-908, 2004), or coexpression of β-1,4-N-acetylglucosaminyltransferase III and Golgi α-mannosidase II or a potent alpha-mannosidase I inhibitor, kifunensine (Ferrara et al., J Biol Chem 281:5032-5036, 2006, Ferrara et al., Biotechnol Bioeng 93:851-861, 2006; Xhou et al., Biotechnol Bioeng 99:652-65, 2008).

The antigen binding domains conjugated to the Ig constant regions or to the fragments of the Ig constant region of the disclosure may have a biantennary glycan structure with fucose content of about between 1% to about 15%, for example about 15%, 14%, 13%, 12%, 11% 10%, 9%, 8%, 7%, 6%, 5%, 4%, 3%, 2% or 1%. Alternatively, the antigen binding domains conjugated to the Ig constant regions or to the fragments of the Ig constant regions may have a glycan structure with fucose content of about 50%, 40%, 45%, 40%, 35%, 30%, 25%, or 20%.

“Fucose content” means the amount of the fucose monosaccharide within the sugar chain at Asn297. The relative amount of fucose is the percentage of fucose-containing structures related to all glycostructures. These may be characterized and quantified by multiple methods, for example: 1) using MALDI-TOF of N-glycosidase F treated sample (e.g. complex, hybrid and oligo- and high-mannose structures) as described in Int Pat. Publ. No. WO2008/077546 2); 2) by enzymatic release of the Asn297 glycans with subsequent derivatization and detection/quantitation by HPLC (UPLC) with fluorescence detection and/or HPLC-MS (UPLC-MS); 3) intact protein analysis of the native or reduced mAb, with or without treatment of the Asn297 glycans with Endo S or other enzyme that cleaves between the first and the second GlcNAc monosaccharides, leaving the fucose attached to the first GlcNAc; 4) digestion of the mAb to constituent peptides by enzymatic digestion (e.g., trypsin or endopeptidase Lys-C), and subsequent separation, detection and quantitation by HPLC-MS (UPLC-MS); 5) Separation of the mAb oligosaccharides from the mAb protein by specific enzymatic deglycosylation with PNGase F at Asn 297. The oligosaccharides thus released can be labeled with a fluorophore, separated and identified by various complementary techniques which allow: fine characterization of the glycan structures by matrix-assisted laser desorption ionization (MALDI) mass spectrometry by comparison of the experimental masses with the theoretical masses, determination of the degree of sialylation by ion exchange HPLC (GlycoSep C), separation and quantification of the oligosaccharide forms according to hydrophilicity criteria by normal-phase HPLC (GlycoSep N), and separation and quantification of the oligosaccharides by high performance capillary electrophoresis-laser induced fluorescence (HPCE-LIF).

“Low fucose” or “low fucose content” as used herein refers to the antigen binding domains conjugated to the Ig constant regions or to the fragments of the Ig constant regions with fucose content of about between 1%-15%.

“Normal fucose” or “normal fucose content” as used herein refers to the antigen binding domains conjugated to the Ig constant regions or to the fragments of the Ig constant regions with fucose content of about over 50%, typically about over 80% or over 85%.

### Polynucleotides, Host Cells and Vectors

The disclosure also provides an isolated polynucleotide, or a combination of polynucleotides encoding any of the multispecific antibodies of the disclosure. These multispecific antibodies comprise the antigen binding domains that bind CD33 and the antigen binding domains that bind the Vδ2 chain of the Vγ9Vδ2 T cell receptor.

The invention also provides an isolated polynucleotide encoding any of CD33 binding antibodies or fragments thereof.

The invention also provides an isolated polynucleotide encoding the VH of SEQ ID NOs: 49, 51, 53, or 55.

The invention also provides an isolated polynucleotide encoding the VL of SEQ ID NOs: 50, 52, 54, or 56.

Some embodiments of the disclosure also provide an isolated or purified nucleic acid comprising a polynucleotide which is complementary to the polynucleotides encoding the CD33 and Vδ2 binding bispecific antibodies of the disclosure or polynucleotides which hybridize under stringent conditions to the polynucleotides encoding the CD33 and Vδ2 binding bispecific antibodies of the disclosure.

The polynucleotides which hybridize under stringent conditions may hybridize under high stringency conditions. By “high stringency conditions” is meant that the polynucleotide specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-12 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand. It is generally appreciated that conditions can be rendered more stringent by the addition of increasing amounts of formamide.

The polynucleotide sequences of the disclosure may be operably linked to one or more regulatory elements, such as a promoter or enhancer, that allow expression of the nucleotide sequence in the intended host cell. The polynucleotide may be a cDNA. The promoter bay be a strong, weak, tissue-specific, inducible or developmental-specific promoter. Exemplary promoters that may be used are hypoxanthine phosphoribosyl transferase (HPRT), adenosine deaminase, pyruvate kinase, beta-actin, human myosin, human hemoglobin, human muscle creatine, and others. In addition, many viral promoters function constitutively in eukaryotic cells and are suitable for use with the described embodiments. Such viral promoters include Cytomegalovirus (CMV) immediate early promoter, the early and late promoters of SV40, the Mouse Mammary Tumor Virus (MMTV) promoter, the long terminal repeats (LTRs) of Maloney leukemia virus, Human Immunodeficiency Virus (HIV), Epstein Barr Virus (EBV), Rous Sarcoma Virus (RSV), and other retroviruses, and the thymidine kinase promoter of Herpes Simplex Virus. Inducible promoters such as the metallothionein promoter, tetracycline-inducible promoter, doxycycline-inducible promoter, promoters that contain one or more interferon-stimulated response elements (ISRE) such as protein kinase R 2′,5′-oligoadenylate synthetases, Mx genes, ADARI, and the like may also be used.

The invention also provides a vector comprising the polynucleotide(s) of the invention. The disclosure also provides an expression vector comprising the polynucleotides of the invention. Such vectors may be plasmid vectors, viral vectors, vectors for baculovirus expression, transposon-based vectors or any other vector suitable for introduction of the synthetic polynucleotides of the invention into a given organism or genetic background by any means. Polynucleotides encoding the CD33 and Vδ2 binding antibodies of the disclosure may be operably linked to control sequences in the expression vector(s) that ensure the expression of the CD33 and Vδ2 binding antibodies. Such regulatory elements may include a transcriptional promoter, sequences encoding suitable mRNA ribosomal binding sites, and sequences that control the termination of transcription and translation. Expression vectors may also include one or more non-transcribed elements such as an origin of replication, a suitable promoter and enhancer linked to the gene to be expressed, other 5′ or 3′ flanking non-transcribed sequences, 5′ or 3′ non-translated sequences (such as necessary ribosome binding sites), a polyadenylation site, splice donor and acceptor sites, or transcriptional termination sequences. An origin of replication that confers the ability to replicate in a host may also be incorporated.

The expression vectors can comprise naturally-occurring or non-naturally-occurring internucleotide linkages, or both types of linkages. The non-naturally occurring or altered nucleotides or internucleotide linkages do not hinder the transcription or replication of the vector.

Once the vector has been incorporated into the appropriate host, the host is maintained under conditions suitable for high level expression of the CD33 and Vδ2 binding antibodies of the disclosure encoded by the incorporated polynucleotides. The transcriptional and translational control sequences in expression vectors to be used in transforming vertebrate cells may be provided by viral sources. Exemplary vectors may be constructed as described by Okayama and Berg, 3 Mol. Cell. Biol. 280 (1983).

Vectors of the disclosure may also contain one or more Internal Ribosome Entry Site(s) (IRES). Inclusion of an IRES sequence into fusion vectors may be beneficial for enhancing expression of some antibodies. In some embodiments, the vector system will include one or more polyadenylation sites (e.g., SV40), which may be upstream or downstream of any of the aforementioned nucleic acid sequences. Vector components may be contiguously linked or arranged in a manner that provides optimal spacing for expressing the gene products (i.e., by the introduction of “spacer” nucleotides between the ORFs) or positioned in another way. Regulatory elements, such as the IRES motif, may also be arranged to provide optimal spacing for expression.

Vectors of the disclosure may be circular or linear. They may be prepared to contain a replication system functional in a prokaryotic or eukaryotic host cell. Replication systems can be derived, e.g., from ColE1, SV40, 2μ plasmid, λ, bovine papilloma virus, and the like.

The recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine deaminase, purine nucleoside phosphorylase, and nitroreductase.

The vectors may also comprise selection markers, which are well known in the art. Selection markers include positive and negative selection marker. Marker genes include biocide resistance, e.g., resistance to antibiotics, heavy metals, etc., complementation in an auxotrophic host to provide prototrophy, and the like. Exemplary marker genes include antibiotic resistance genes (e.g., neomycin resistance gene, a hygromycin resistance gene, a kanamycin resistance gene, a tetracycline resistance gene, a penicillin resistance gene, histidinol resistance gene, histidinol x resistance gene), glutamine synthase genes, HSV-TK, HSV-TK derivatives for ganciclovir selection, or bacterial purine nucleoside phosphorylase gene for 6-methylpurine selection (Gadi et al., 7 Gene Ther. 1738-1743 (2000)). A nucleic acid sequence encoding a selection marker or the cloning site may be upstream or downstream of a nucleic acid sequence encoding a polypeptide of interest or cloning site.

Exemplary vectors that may be used are Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia), pEE6.4 (Lonza) and pEE12.4 (Lonza). Additional vectors include the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λEMBL4, and λNM1149, λZapII (Stratagene) can be used. Exemplary plant expression vectors include pBI01, pBI01.2, pBI121, pBI101.3, and pBIN19 (Clontech). Exemplary animal expression vectors include pEUK-Cl, pMAM, and pMAMneo (Clontech). The expression vector may be a viral vector, e.g., a retroviral vector, e.g., a gamma retroviral vector.

The vector may comprise (i) the polynucleotide encoding the VH of SEQ ID NOs: 49, 51, 53, or 54; (ii) the polynucleotide encoding the VL of SEQ ID NO: 50, 52, 54, or 56; (iii) the polynucleotide encoding the VHH of SEQ ID NO: 57 or 58; (iv) or any combination thereof. These polynucleotides may be co-expressed in the same host cell from one or more expression vectors.

In some embodiments, the vector comprises a polynucleotide encoding for a polypeptide comprising:
a) the VH of SEQ ID NO: 49 and the VL of SEQ ID NO: 50;
b) the VH of SEQ ID NO: 51 and the VL of SEQ ID NO: 52;
c) the VH of SEQ ID NO: 53 and the VL of SEQ ID NO: 54;
d) the VH of SEQ ID NO: 55 and the VL of SEQ ID NO: 56;
e) the linker of any of SEQ ID NO: 67-100;
f) the ScFv of SEQ ID NO: 130-133;
g) the VHH of SEQ ID NO: 57 or 58;
h) the Fc region of SEQ ID NO: 128; or
i) any combination thereof.

The invention also provides for a host cell comprising one or more vectors of the invention. “Host cell” refers to a cell into which a vector has been introduced. It is understood that the term host cell is intended to refer not only to the particular subject cell but to the progeny of such a cell, and also to a stable cell line generated from the particular subject cell. Because certain modifications may occur in succeeding generations due to either mutation or environmental influences, such progeny may not be identical to the parent cell, but are still included within the scope of the term “host cell” as used herein. Such host cells may be eukaryotic cells, prokaryotic cells, plant cells or archeal cells. Escherichia coli, bacilli, such as Bacillus subtilis, and other enterobacteriaceae, such as Salmonella, Serratia, and various Pseudomonas species are examples of prokaryotic host cells. Other microbes, such as yeast, are also useful for expression. Saccharomyces (e.g., S. cerevisiae) and Pichia are examples of suitable yeast host cells. Exemplary eukaryotic cells may be of mammalian, insect, avian or other animal origins. Mammalian eukaryotic cells include immortalized cell lines such as hybridomas or myeloma cell lines such as SP2/0 (American Type Culture Collection (ATCC), Manassas, VA, CRL-1581), NS0 (European Collection of Cell Cultures (ECACC), Salisbury, Wiltshire, UK, ECACC No. 85110503), FO (ATCC CRL-1646) and Ag653 (ATCC CRL-1580) murine cell lines. An exemplary human myeloma cell line is U266 (ATTC CRL-TIB-196). Other useful cell lines include those derived from Chinese Hamster Ovary (CHO) cells such as CHO-KISV (Lonza Biologics, Walkersville, MD), CHO-K1 (ATCC CRL-61) or DG44.

The disclosure also provides a method of producing the antibodies of the disclosure comprising culturing the host cell of the disclosure in conditions that the K2 binding protein is expressed, and recovering the antibodies produced by the host cell. Methods of making proteins and purifying them are known. Once synthesized (either chemically or recombinantly), the antibodies may be purified according to standard procedures, including ammonium sulfate precipitation, affinity columns, column chromatography, high performance liquid chromatography (HPLC) purification, gel electrophoresis, and the like (see generally Scopes, Protein Purification (Springer-Verlag, N.Y., (1982)). A subject protein may be substantially pure, e.g., at least about 80% to 85% pure, at least about 85% to 90% pure, at least about 90% to 95% pure, or at least about 98% to 99%, or more, pure, e.g., free from contaminants such as cell debris, macromolecules, etc. other than the subject protein.

The polynucleotides encoding the antibodies of the disclosure may be incorporated into vectors using standard molecular biology methods. Host cell transformation, culture, antibody expression and purification are done using well known methods. Accordingly, the invention provides a method of producing a polypeptide comprising expressing a nucleotide of the invention that encodes for a polypeptide of the invention.

Where the polypeptide is formed of separate chains which are encoded by different nucleic acids, the invention provides a method of producing a polypeptide comprising expressing nucleotides of the invention that encode for a polypeptide of the invention.

A first polynucleotide of the invention may encode a polypeptide comprising the VH of SEQ ID NO: 49 and a heavy chain constant region, a second polynucleotide of the invention may encode a polypeptide comprising the VL of SEQ ID NO: 50 and a light chain constant region, and a third polynucleotide of the invention may encode a polypeptide comprising the VHH of SEQ ID NO: 58 and an Fc polypeptide.

A first polynucleotide of the invention may encode a polypeptide comprising the VH of SEQ ID NO: 51 and a heavy chain constant region, a second polynucleotide of the invention may encode a polypeptide comprising the VL of SEQ ID NO: 52 and a light chain constant region, and a third polynucleotide of the invention may encode a polypeptide comprising the VHH of SEQ ID NO: 58 and an Fc polypeptide.

A first polynucleotide of the invention may encode a polypeptide comprising the VH of SEQ ID NO: 53 and a heavy chain constant region, a second polynucleotide of the invention may encode a polypeptide comprising the VL of SEQ ID NO: 54 and a light chain constant region, and a third polynucleotide of the invention may encode a polypeptide comprising the VHH of SEQ ID NO: 58 and an Fc polypeptide.

A first polynucleotide of the invention may encode a polypeptide comprising the VH of SEQ ID NO: 55 and a heavy chain constant region, a second polynucleotide of the invention may encode a polypeptide comprising the VL of SEQ ID NO: 56 and a light chain constant region, and a third polynucleotide of the invention may encode a polypeptide comprising the VHH of SEQ ID NO: 58 and an Fc polypeptide.

A first polynucleotide of the invention may encode a polypeptide comprising the VH of SEQ ID NO: 49 and a heavy chain constant region, a second polynucleotide of the invention may encode a polypeptide comprising the VL of SEQ ID NO: 50 and a light chain constant region, and a third polynucleotide of the invention may encode a polypeptide comprising the VHH of SEQ ID NO: 57 and an Fc polypeptide.

A first polynucleotide of the invention may encode a polypeptide comprising the VH of SEQ ID NO: 51 and a heavy chain constant region, a second polynucleotide of the invention may encode a polypeptide comprising the VL of SEQ ID NO: 52 and a light chain constant region, and a third polynucleotide of the invention may encode a polypeptide comprising the VHH of SEQ ID NO: 57 and an Fc polypeptide.

A first polynucleotide of the invention may encode a polypeptide comprising the VH of SEQ ID NO: 53 and a heavy chain constant region, a second polynucleotide of the invention may encode a polypeptide comprising the VL of SEQ ID NO: 54 and a light chain constant region, and a third polynucleotide of the invention may encode a polypeptide comprising the VHH of SEQ ID NO: 57 and an Fc polypeptide.

A first polynucleotide of the invention may encode a polypeptide comprising the VH of SEQ ID NO: 55 and a heavy chain constant region, a second polynucleotide of the invention may encode a polypeptide comprising the VL of SEQ ID NO: 56 and a light chain constant region, and a third polynucleotide of the invention may encode a polypeptide comprising the VHH of SEQ ID NO: 57 and an Fc polypeptide.

Modified nucleotides may be used to generate the polynucleotides of the disclosure. Exemplary modified nucleotides are 5-fluorouracil, 5-bromouracil, 5-chlorouracil, 5-iodouracil, hypoxanthine, xanthine, 4-acetylcytosine, 5-(carboxyhydroxymethyl) uracil, carboxymethylaminomethyl-2-thiouridine, 5-carboxymethylaminomethyluracil, dihydrouracil, N⁶-substituted adenine, 7-methylguanine, 5-methylaminomethyluracil, 5-methoxyaminomethyl-2-thiouracil, beta-D-mannosylqueosine, 5″-methoxycarboxymethyluracil, 5-methoxyuracil, 2-methylthio-N6-isopentenyladenine, uracil-5-oxyacetic acid (v), wybutoxosine, pseudouracil, queuosine, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine.

### Medical Uses & Methods of Treatment

The isolated multispecific or bispecific antibodies of the disclosure may be used as a medicament, in particular for the treatment of cancer.

Exemplary cancers that are amenable to treatment by the bispecific CD33/82 antibodies of the invention include hematologic cancers selected from the group consisting of leukemia, lymphoma, multiple myeloma, acute myeloid leukemia (AML), myelodysplastic syndrome (MDS), acute lymphocytic leukemia (ALL), diffuse large B-cell lymphoma (DLBCL), chronic myeloid leukemia (CML), blastic plasmacytoid dendritic cell neoplasm (DPDCN), myeloproliferative neoplasm (MPNs), and mixed phenotype acute leukemia.

Another aspect of the invention is the multispecific or bispecific antibody as defined in the claims for use in a method of treating a subject having cancer, comprising administering a therapeutically effective amount of the isolated bispecific CD33/82 antibody of the invention to a patient in need thereof for a time sufficient to treat the cancer.

### Administration/Pharmaceutical Compositions

The disclosure provides for pharmaceutical compositions comprising the multispecific or bispecific CD33/82 antibody as disclosed herein and a pharmaceutically acceptable carrier or excipient. For therapeutic use, the multispecific or bispecific CD33/82 antibodies of the invention may be prepared as pharmaceutical compositions containing an effective amount of the antibody as an active ingredient in a pharmaceutically acceptable carrier. The term “carrier” refers to a diluent, adjuvant, excipient, or vehicle with which the active compound is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the molecules of the disclosure or antibodies of the invention in such pharmaceutical formulation may vary widely, i.e., from less than about 0.5%, usually to at least about 1% to as much as 15 or 20% by weight and will be selected primarily based on required dose, fluid volumes, viscosities, etc., according to the particular mode of administration selected. Suitable vehicles and formulations, inclusive of other human proteins, e.g., human serum albumin, are described, for example, in e.g. Remington: The Science and Practice of Pharmacy, 21st Edition, Troy, D. B. ed., Lipincott Williams and Wilkins, Philadelphia, PA 2006, Part 5, Pharmaceutical Manufacturing pp 691-1092, See especially pp. 958-989.

The mode of administration for therapeutic use of the bispecific CD33/82 antibodies of the invention may be any suitable route that delivers the agent to the host, such as parenteral administration, e.g., intradermal, intramuscular, intraperitoneal, intravenous or subcutaneous, pulmonary, transmucosal (oral, intranasal, intravaginal, rectal), using a formulation in a tablet, capsule, solution, powder, gel, particle; and contained in a syringe, an implanted device, osmotic pump, cartridge, micropump; or other means appreciated by the skilled artisan, as well known in the art. Site specific administration may be achieved by for example intrarticular, intrabronchial, intraabdominal, intracapsular, intracartilaginous, intracavitary, intracelial, intracerebellar, intracerebroventricular, intracolic, intracervical, intragastric, intrahepatic, intracardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal, or transdermal delivery.

Thus, a pharmaceutical composition of the invention for intramuscular injection may be prepared to contain 1 ml sterile buffered water, and between about 1 ng to about 100 mg/kg, e.g. about 50 ng to about 30 mg/kg or more preferably, about 5 mg to about 25 mg/kg, of the bispecific CD33/82 antibodies of the invention.

The bispecific CD33/82 antibodies of the invention may be administered to a patient by any suitable route, for example parentally by intravenous (IV) infusion or bolus injection, intramuscularly or subcutaneously or intraperitoneally. IV infusion can be given over as little as 15 minutes, but more often for 30 minutes, 60 minutes, 90 minutes or even 2, 3, 4, 5, 6 or 7 hours. The bispecific CD33/82 antibodies of the invention may also be injected directly into the site of disease (e.g., the tumor itself). The dose given to a patient having a cancer is sufficient to alleviate or at least partially arrest the disease being treated (“therapeutically effective amount”) and may be sometimes 0.1 to 10 mg/kg body weight, for example 1, 2, 3, 4, 5, 6, 7, 8, 9 or 10 mg/kg, but may even higher, for example 15, 20, 30, 40, 50, 60, 70, 80, 90 or 100 mg/kg. A fixed unit dose may also be given, for example, 50, 100, 200, 500 or 1000 mg, or the dose may be based on the patient's surface area, e.g., 400, 300, 250, 200, or 100 mg/m². Usually between 1 and 8 doses, (e.g., 1, 2, 3, 4, 5, 6, 7 or 8) may be administered to treat cancer, but 10, 12, 20 or more doses may be given. Administration of the bispecific CD33/82 antibody of the invention may be repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration may be at the same dose or at a different dose.

For example, a pharmaceutical composition comprising the bispecific CD33/82 antibody of the invention for intravenous infusion may be made up to contain about 200 ml of sterile Ringer's solution, and about 8 mg to about 2400 mg, about 400 mg to about 1600 mg, or about 400 mg to about 800 mg of the bispecific CD33/82 antibody for administration to an 80-kg patient. Methods for preparing parenterally administrable compositions are well known and are described in more detail in, for example, “Remington's Pharmaceutical Science”, 15th ed., Mack Publishing Company, Easton, PA.

The bispecific CD33/82 antibodies of the invention may be lyophilized for storage and reconstituted in a suitable carrier prior to use. This technique has been shown to be effective with conventional protein preparations and well known lyophilization and reconstitution techniques can be employed.

The bispecific CD33/82 antibodies of the invention may be administered in combination with a second therapeutic agent simultaneously, sequentially or separately. The second therapeutic agent may be a chemotherapeutic agent or a targeted anti-cancer therapy.

The bispecific CD33/82 antibody may be administered together with any one or more of the chemotherapeutic drugs or other anti-cancer therapeutics known to those of skill in the art.

When the bispecific CD33/82 antibody of the invention is administered in combination with a second therapeutic agent, the combination may take place over any convenient timeframe. For example, the bispecific CD33/82 antibody and the second therapeutic agent may be administered to a patient on the same day, and even in the same intravenous infusion. However, the bispecific CD33/82 antibody and the second therapeutic agent may also be administered on alternating days or alternating weeks, fortnights or months, and so on. In some methods, the bispecific CD33/82 antibody and the second therapeutic agent are administered with sufficient proximity in time that they are simultaneously present (e.g., in the serum) at detectable levels in the patient being treated. In some methods, an entire course of treatment of the bispecific CD33/82 antibody consisting of a number of doses over a time period is followed or preceded by a course of treatment of the second therapeutic agent also consisting of a number of doses. In some methods, treatment with the bispecific CD33/82 antibody administered second is begun if the patient has resistance or develops resistance to the second therapeutic agent administered initially. The patient may receive only a single course or multiple courses of treatment with one or both the bispecific CD33/82 antibody and the second therapeutic agent. A recovery period of 1, 2 or several days or weeks may be used between administration of the bispecific CD33/82 antibody and the second therapeutic agent. When a suitable treatment regimen has already been established for the second therapeutic agent, that regimen may be used in combination with the bispecific CD33/82 antibody of the invention.

The bispecific CD33/82 antibody, optionally in combination with the second therapeutic agent may be administered together with any form of radiation therapy including external beam radiation, intensity modulated radiation therapy (IMRT) and any form of radiosurgery including Gamma Knife, Cyberknife, Linac, and interstitial radiation (e.g. implanted radioactive seeds, GliaSite balloon), and/or with surgery.

Table 3 shows the amino acid sequences of the CDR sequences of the JLSantibody (an antibody having the VH of SEQ ID NO: 49 and the VL of SEQ ID NO: 50) as defined according to the AbM, Kabat, Chothia, IMGT and Contact systems.

TABLE 3

| | HC1 | HC2 | HC3 | LC1 | LC2 | LC3 |
|---|---|---|---|---|---|---|
| AbM | GASIR | HIYSTGNIH | DNGAALFD | SGSSSNIGSNI | SNNQRPS | AAWDDSLN |
| | NYYWS | (SEQ ID NO: | Y (SEQ ID | VN (SEQ ID | (SEQ ID | GPV (SEQ ID |
| | (SEQ ID | 135) | NO: 136) | NO: 137) | NO: 138) | NO: 139) |
| | NO: 134) | | | | | |
| Kabat | NYYWS | HIYSTGNIHYN | DNGAALFD | SGSSSNIGSNI | SNNQRPS | AAWDDSLN |
| | (SEQ ID | PSLKS (SEQ | Y (SEQ ID | VN (SEQ ID | (SEQ ID | GPV (SEQ ID |
| | NO: 1) | ID NO: 2) | NO: 3) | NO: 4) | NO: 5) | NO: 6) |
| Chothia | GASIR | YSTGN (SEQ | DNGAALFD | SSSNIGSNI | SNN (SEQ | WDDSLNGP |
| | NY | ID NO: 141) | (SEQ ID | (SEQ ID NO: | ID NO: | (SEQ ID NO: |
| | (SEQ ID | | NO: 142) | 143) | 144) | 145) |
| | NO: 140) | | | | | |
| IMGT | GASIR | IYSTGNI | ARDNGAAL | SSNIGSNI | SNN (SEQ | AAWDDSLN |
| | NYY | (SEQ ID | FDY (SEQ | (SEQ ID NO: | ID NO: | GPV (SEQ ID |
| | (SEQ ID | NO: 147) | ID NO: | 149) | 150) | NO: 151) |
| | NO: 146) | | 148) | | | |
| Contact | RNYY | WLGHIYSTGN | ARDNGAAL | IGSNIVNWY | LLIYSNNQ | AAWDDSLN |
| | WS | IH (SEQ ID | FD (SEQ | (SEQ ID NO: | RP (SEQ ID | GP (SEQ ID |
| | (SEQ ID | NO: 153) | ID NO: | 155) | NO: 156) | NO: 157) |
| | NO: 152) | | 154) | | | |

Table 4 shows the amino acid sequences of the CDR sequences of the JL6 antibody (an antibody having the VH of SEQ ID NO: 51 and the VL of SEQ ID NO: 52) as defined according to the AbM, Kabat, Chothia, IMGT and Contact systems.

TABLE 4

| | HC1 | HC2 | HC3 | LC1 | LC2 | LC3 |
|---|---|---|---|---|---|---|
| AbM | GGSISSYY | YIYYSGSTN | MWEILGF | SGSSSNIGSN | SNNQRPS | AAWDDSL |
| | WG (SEQ ID | (SEQ ID NO: | DP (SEQ ID | PVN (SEQ ID | (SEQ ID | NGPV (SEQ |
| | NO: 158) | 159) | NO: 160) | NO: 161) | NO: 162) | ID NO: 163) |
| Kabat | SYYWG | YIYYSGSTNY | MWEILGF | SGSSSNIGSN | SNNQRPS | AAWDDSL |
| | (SEQ ID NO: | NPSLKS (SEQ | DP (SEQ ID | PVN (SEQ ID | (SEQ ID | NGPV (SEQ |
| | 7) | ID NO: 8) | NO: 9) | NO: 10) | NO: 11) | ID NO: 12) |
| Chothia | GGSISSY | YYSGS (SEQ | MWEILGF | SSSNIGSNP | | WDDSLNG |
| | (SEQ ID NO: | ID NO: 165) | D (SEQ ID | (SEQ ID NO: | SNN (SEQ | P (SEQ ID |
| | 164) | | NO: 166) | 167) | ID NO: 168) | NO: 169) |
| IMGT | GGSISSYY | IYYSGST | ARMWEIL | SSNIGSNP | | AAWDDSL |
| | (SEQ ID NO: | (SEQ ID NO: | GFDP (SEQ | (SEQ ID NO: | SNN (SEQ | NGPV (SEQ |
| | 170) | 171) | ID NO: 172) | 173) | ID NO: 174) | ID NO: 175) |
| Contact | SSYYWG | WIGYIYYSGS | ARMWEIL | IGSNPVNWY | LLIYSNNQ | AAWDDSL |
| | (SEQ ID NO: | TN (SEQ ID | GFD (SEQ | (SEQ ID NO: | RP (SEQ ID | NGP (SEQ |
| | 176) | NO: 177) | ID NO: 178) | 179) | NO: 180) | ID NO: 181) |

Table 5 shows the amino acid sequences of the CDR sequences of the JL2 antibody (an antibody having the VH of SEQ ID NO: 53 and the VL of SEQ ID NO: 54) as defined according to the AbM, Kabat, Chothia, IMGT and Contact systems.

TABLE 5

| | HC1 | HC2 | HC3 | LC1 | LC2 | LC3 |
|---|---|---|---|---|---|---|
| AbM | GFTFSSY | NIKQDGSER | EVGYNWN | RSSQSLLHSD | LGSYRA | MQVLQTPWT |
| | WMT (SEQ | Y (SEQ | QGGYFDY | GYNYLD (SEQ | S (SEQ ID | (SEQ ID |
| | ID NO: | ID NO: | (SEQ ID | ID NO: 185) | NO: 186) | NO: 187) |
| | 182) | 183) | NO: 184) | | | |
| Kabat | SYWMT | NIKQDGSER | EVGYNWN | RSSQSLLHSD | LGSYRA | MQVLQTPWT |
| | (SEQ ID | YYVDSVKG | QGGYFDY | GYNYLD (SEQ | S (SEQ ID | (SEQ ID |
| | NO: 13) | (SEQ ID | (SEQ ID | ID NO: 16) | NO: 17) | NO: 18) |
| | | NO: 14) | NO: 15) | | | |
| Chothia | GFTFSSY | KQDGSE | EVGYNWN | SQSLLHSDG | LGS (SEQ | VLQTPW |
| | (SEQ ID | (SEQ ID | QGGYFD | YNY (SEQ ID | ID NO: | (SEQ ID |
| | NO: 188) | NO: 189) | (SEQ ID | NO: 191) | 192) | NO: 193) |
| | | | NO: 190) | | | |
| IMGT | GFTFSSYW | IKQDGSER | AREVGYNW | QSLLHSDGY | LGS (SEQ | MQVLQTPWT |
| | (SEQ ID | (SEQ ID | NQGGYFDY | NY (SEQ ID | ID NO: | (SEQ ID |
| | NO: 194) | NO: 195) | (SEQ ID | NO: 197) | 198) | NO: 199) |
| | | | NO: 196) | | | |
| Contact | SSYWMT | WVANIKQD | AREVGYN | LHSDGYNYL | LLIYLG | MQVLQTPW |
| | (SEQ ID | GSERY | WNQGGYFD | DWY (SEQ ID | SYRA | (SEQ ID |
| | NO: 200) | (SEQ ID | (SEQ ID | NO: 203) | (SEQ ID | NO: 205) |
| | | NO: 201) | NO: 202) | | NO: 204) | |

Table 6 shows the amino acid sequences of the CDR sequences of the JL3 antibody (an antibody having the VH of SEQ ID NO: 55 and the VL of SEQ ID NO: 56) as defined according to the AbM, Kabat, Chothia, IMGT and Contact systems.

TABLE 6

| | HC1 | HC2 | HC3 | LC1 | LC2 | LC3 |
|---|---|---|---|---|---|---|
| AbM | GGSIR | HIFSTGHIN | DNGAALFD | SGSSSNIGSN | SDNQRPS | AAWDDSLN |
| | NYYWS | (SEQ ID | F (SEQ ID | IVN (SEQ ID | (SEQ ID NO: | GPV (SEQ ID |
| | (SEQ ID | NO: 207) | NO: 208) | NO: 209) | 210) | NO: 211) |
| | NO: 206) | | | | | |
| Kabat | NYYWS | HIFSTGHIN | DNGAALFD | SGSSSNIGSN | SDNQRPS | AAWDDSLN |
| | (SEQ ID | YDSSLKS | F (SEQ ID | IVN (SEQ ID | (SEQ ID NO: | GPV (SEQ ID |
| | NO: 19) | (SEQ ID | NO: 21) | NO: 22) | 23) | NO: 24) |
| | | NO: 20) | | | | |
| Chothia | GGSIR | FSTGH | DNGAALFD | SSSNIGSNI | SDN (SEQ | WDDSLNGP |
| | NY (SEQ | (SEQ ID | (SEQ ID | (SEQ ID NO: | ID NO: 216) | (SEQ ID NO: |
| | ID NO: | NO: 213) | NO: 214) | 215) | | 217) |
| | 212) | | | | | |
| IMGT | GGSIR | IFSTGHI | ARDNGAALF | SSNIGSNI | SDN (SEQ | AAWDDSLN |
| | NYY | (SEQ ID | DF (SEQ ID | (SEQ ID NO: | ID NO: 222) | GPV (SEQ ID |
| | (SEQ ID | NO: 219) | NO: 220) | 221) | | NO: 223) |
| | NO: 218) | | | | | |
| Contact | RNYY | WFGHIFSTG | ARDNGAALF | IGSNIVNWY | LLLYSDN | AAWDDSLN |
| | WS | HIN (SEQ | D (SEQ ID | (SEQ ID NO: | QRP (SEQ | GP (SEQ ID |
| | (SEQ ID | ID NO: | NO: 226) | 227) | ID NO: 228) | NO: 229) |
| | NO: 224) | 225) | | | | |

While having described the invention in general terms, the embodiments of the invention will be further disclosed in the following examples that should not be construed as limiting the scope of the claims.

### EXAMPLES

### Example 1. CD33 Antigen Generation

Expression constructs encoding the human CD33 extracellular domain (ECD) or its sub-domains were designed based on the sequence of myeloid cell surface antigen CD33 (Uniprot accession #P20138) and its domain annotation with either 6X His-tag sequence (SEQ ID NO: 246) or as a fusion protein to a C34S variant of human serum albumin (HSA) with a 6X His-tag sequence (SEQ ID NO: 246) at the C-terminus. Similar expression constructs encoding CD33 (ECD) or its sub-domains from cynomolgus monkey (Macaca fascicularis) were designed based on NCBI Accession #XP_005590138.1. The amino acid sequences of the generated antigens are shown in Table 7.

Human and cyno CD33 full-length ECD or sub-domain expression constructs were 5 transiently transfected into HEK293 derived cells, Expi293 (Gibco/Thermo Fisher Scientific) using Expifectamine according to the manufacturer's protocol. Cells were incubated 5 days at 37° C. with 8% CO₂ on an orbital shaker before harvesting. The cells expressing the protein were removed by centrifugation and the soluble CD33 proteins with his-tags were purified from the media using immobilized metal-ion affinity chromatography (IMAC) using Ni NTA Sepharose 6 Fast Flow resin (GE Healthcare) and subsequently buffer-exchanged into 1X Dubelcco's Phosphate Saline buffer pH 7.2 without calcium or magnesium using Zeba™ Spin Desalting Columns, 7K MWCO, 10 mL; ThermoScientific Catalog number: 89893 per the manufacturer's specifications.

TABLE 7
Amino acid sequences of the CD33 antigens.

| Protein | | |
|---|---|---|
| AA ID | Description | Amino Acid Sequence |
| C33W1 | HSA N-terminal | DPRVRLEVQESVTVQEGLCVLVPCTFFHPVPYHTRNSPVHG |
| | Fusion; | YWFREGAIVSLDSPVATNKLDQEVQEETQGRFRLLGDPSRN |
| | CynoCD33ECD- | NCSLSIVDARRRDNGSYFFRMEKGSTKYSYKSTQLSVHVTD |
| | HSA(C34S)-6xHis | LTHRPQILIPGALDPDHSKNLTCSVPWACEQGTPPIFSWMSA |
| | | APTSLGLRTTHSSVLIITPRPQDHGTNLTCQVKFPGAGVTTE |
| | | RTIQLNVSYASQNPRTDIFLGDGSGKQGVVQGSGSGSENLY |
| | | FQGVRSSSDAHKSEVAHRFKDLGEENFKALVLIAFAQYLQQ |
| | | SPFEDHVKLVNEVTEFAKTCVADESAENCDKSLHTLFGDKL |
| | | CTVATLRETYGEMADCCAKQEPERNECFLQHKDDNPNLPR |
| | | LVRPEVDVMCTAFHDNEETFLKKYLYEIARRHPYFYAPELL |
| | | FFAKRYKAAFTECCQAADKAACLLPKLDELRDEGKASSAK |
| | | QRLKCASLQKFGERAFKAWAVARLSQRFPKAEFAEVSKLV |
| | | TDLTKVHTECCHGDLLECADDRADLAKYICENQDSISSKLK |
| | | ECCEKPLLEKSHCIAEVENDEMPADLPSLAADFVESKDVCK |
| | | NYAEAKDVFLGMFLYEYARRHPDYSVVLLLRLAKTYETTL |
| | | EKCCAAADPHECYAKVFDEFKPLVEEPQNLIKQNCELFEQL |
| | | GEYKFQNALLVRYTKKVPQVSTPTLVEVSRNLGKVGSKCC |
| | | KHPEAKRMPCAEDYLSVVLNQLCVLHEKTPVSDRVTKCCT |
| | | ESLVNRRPCFSALEVDETYVPKEFNAETFTFHADICTLSEKE |
| | | RQIKKQTALVELVKHKPKATKEQLKAVMDDFAAFVEKCCK |
| | | ADDKETCFAEEGKKLVAASQAALGLGGGSHHHHHH |
| | | (SEQ ID NO: 230) |
| C33W2 | HSA N-terminal | DPNFWLQVQESVTVQEGLCVLVPCTFFHPIPYYDKNSPVHG |
| | Fusion; Human | YWFREGAIISRDSPVATNKLDQEVQEETQGRFRLLGDPSRN |
| | CD33ECD-Tev- | NCSLSIVDARRRDNGSYFFRMERGSTKYSYKSPQLSVHVTD |
| | HSA(C34S)-6xHis | LTHRPKILIPGTLEPGHSKNLTCSVSWACEQGTPPIFSWLSAA |
| | | PTSLGPRTTHSSVLIITPRPQDHGTNLTCQVKFAGAGVTTER |
| | | TIQLNVTYVPQNPTTGIFPGDGSGKQETRAGVVHGSGSGSE |
| | | NLYFQGVRSSSDAHKSEVAHRFKDLGEENFKALVLIAFAQY |
| | | LQQSPFEDHVKLVNEVTEFAKTCVADESAENCDKSLHTLFG |
| | | DKLCTVATLRETYGEMADCCAKQEPERNECFLQHKDDNPN |
| | | LPRLVRPEVDVMCTAFHDNEETFLKKYLYEIARRHPYFYAP |
| | | ELLFFAKRYKAAFTECCQAADKAACLLPKLDELRDEGKASS |
| | | AKQRLKCASLQKFGERAFKAWAVARLSQRFPKAEFAEVSK |
| | | LVTDLTKVHTECCHGDLLECADDRADLAKYICENQDSISSK |
| | | LKECCEKPLLEKSHCIAEVENDEMPADLPSLAADFVESKDV |
| | | CKNYAEAKDVFLGMFLYEYARRHPDYSVVLLLRLAKTYET |
| | | TLEKCCAAADPHECYAKVFDEFKPLVEEPQNLIKQNCELFE |
| | | QLGEYKFQNALLVRYTKKVPQVSTPTLVEVSRNLGKVGSK |
| | | CCKHPEAKRMPCAEDYLSVVLNQLCVLHEKTPVSDRVTKC |
| | | CTESLVNRRPCFSALEVDETYVPKEFNAETFTFHADICTLSE |
| | | KERQIKKQTALVELVKHKPKATKEQLKAVMDDFAAFVEKC |
| | | CKADDKETCFAEEGKKLVAASQAALGLGGGSHHHHHH |
| | | (SEQ ID NO: 231) |
| C33W3 | HSA N-terminal | DPNFWLQVQESVTVQEGLCVLVPCTFFHPIPYYDKNSPVHG |
| | Fusion; hCD33 Ig- | YWFREGAIISRDSPVATNKLDQEVQEETQGRFRLLGDPSRN |
| | like V-type domain | NCSLSIVDARRRDNGSYFFRMERGSTKYSYKSPQLSVHVTD |
| | (Uniprot P20138, | LTHRDAHKSEVAHRFKDLGEENFKALVLIAFAQYLQQSPFE |
| | V136-H259) fused | DHVKLVNEVTEFAKTCVADESAENCDKSLHTLFGDKLCTV |
| | to HSA and 6xHis | ATLRETYGEMADCCAKQEPERNECFLQHKDDNPNLPRLVR |
| | Tag (SEQ ID NO: | PEVDVMCTAFHDNEETFLKKYLYEIARRHPYFYAPELLFFA |
| | 246) at C-terminus | KRYKAAFTECCQAADKAACLLPKLDELRDEGKASSAKQRL |
| | | KCASLQKFGERAFKAWAVARLSQRFPKAEFAEVSKLVTDL |
| | | TKVHTECCHGDLLECADDRADLAKYICENQDSISSKLKECC |
| | | EKPLLEKSHCIAEVENDEMPADLPSLAADFVESKDVCKNYA |
| | | EAKDVFLGMFLYEYARRHPDYSVVLLLRLAKTYETTLEKC |
| | | CAAADPHECYAKVFDEFKPLVEEPQNLIKQNCELFEQLGEY |
| | | KFQNALLVRYTKKVPQVSTPTLVEVSRNLGKVGSKCCKHPE |
| | | AKRMPCAEDYLSVVLNQLCVLHEKTPVSDRVTKCCTESLV |
| | | NRRPCFSALEVDETYVPKEFNAETFTFHADICTLSEKERQIK |
| | | KQTALVELVKHKPKATKEQLKAVMDDFAAFVEKCCKADD |
| | | KETCFAEEGKKLVAASQAALGLHHHHHH |
| | | (SEQ ID NO: 232) |
| C33W4 | HSA N-terminal | VHVTDLTHRPKILIPGTLEPGHSKNLTCSVSWACEQGTPPIFS |
| | Fusion; hCD33 Ig- | WLSAAPTSLGPRTTHSSVLIITPRPQDHGTNLTCQVKFAGAG |
| | like C2-type | VTTERTIQLNVTYVPQNPTTGIFPGDGSGKQETRAGVVHDA |
| | domain (Uniprot | HKSEVAHRFKDLGEENFKALVLIAFAQYLQQSPFEDHVKLV |
| | P20138, V136- | NEVTEFAKTCVADESAENCDKSLHTLFGDKLCTVATLRETY |
| | H259) fused to | GEMADCCAKQEPERNECFLQHKDDNPNLPRLVRPEVDVMC |
| | HSA and 6xHis | TAFHDNEETFLKKYLYEIARRHPYFYAPELLFFAKRYKAAFT |
| | Tag (SEQ ID NO: | ECCQAADKAACLLPKLDELRDEGKASSAKQRLKCASLQKF |
| | 246) at C-terminus | GERAFKAWAVARLSQRFPKAEFAEVSKLVTDLTKVHTECC |
| | | HGDLLECADDRADLAKYICENQDSISSKLKECCEKPLLEKSH |
| | | CIAEVENDEMPADLPSLAADFVESKDVCKNYAEAKDVFLG |
| | | MFLYEYARRHPDYSVVLLLRLAKTYETTLEKCCAAADPHE |
| | | CYAKVFDEFKPLVEEPQNLIKQNCELFEQLGEYKFQNALLV |
| | | RYTKKVPQVSTPTLVEVSRNLGKVGSKCCKHPEAKRMPCA |
| | | EDYLSVVLNQLCVLHEKTPVSDRVTKCCTESLVNRRPCFSA |
| | | LEVDETYVPKEFNAETFTFHADICTLSEKERQIKKQTALVEL |
| | | VKHKPKATKEQLKAVMDDFAAFVEKCCKADDKETCFAEE |
| | | GKKLVAASQAALGLHHHHHH |
| | | (SEQ ID NO: 233) |
| C33W8 | hCD33_Ig-like V- | DPNFWLQVQESVTVQEGLCVLVPCTFFHPIPYYDKNSPVHG |
| | Type domain | YWFREGAIISRDSPVATNKLDQEVQEETQGRFRLLGDPSRN |
| | | NCSLSIVDARRRDNGSYFFRMERGSTKYSYKSPQLSVHVTD |
| | | LTHRGSHHHHHH |
| | | (SEQ ID NO: 234) |
| C33W9 | hCD33 Ig-like C2- | VHVTDLTHRPKILIPGTLEPGHSKNLTCSVSWACEQGTPPIFS |
| | type domain | WLSAAPTSLGPRTTHSSVLIITPRPQDHGTNLTCQVKFAGAG |
| | | VTTERTIQLNVTYVPQNPTTGIFPGDGSGKQETRAGVVHGS |
| | | HHHHHH |
| | | (SEQ ID NO: 235) |
| C33W10 | MulgV_Hu IgC2 | QDLEFQLVAPESVTVEEGLCVHVPCSVFYPSIKLTLGPVTGS |
| | chimera CD33- | WLRKGVSLHEDSPVATSDPRQLVQKATQGRFQLLGDPQKH |
| | 6xHis v1 | DCSLFIRDAQKNDTGMYFFRVVREPFVRYSYKKSQLSLHVT |
| | | SLSRTPKILIPGTLEPGHSKNLTCSVSWACEQGTPPIFSWLSA |
| | | APTSLGPRTTHSSVLIITPRPQDHGTNLTCQVKFAGAGVTTE |
| | | RTIQLNVTYVPQNPTTGIFPGDGSGKQETRAGVVHGSHHHH |
| | | HH |
| | | (SEQ ID NO: 236) |
| C33W12 | MulgV_cyno IgC2 | QDLEFQLVAPESVTVEEGLCVHVPCSVFYPSIKLTLGPVTGS |
| | chimera CD33- | WLRKGVSLHEDSPVATSDPRQLVQKATQGRFQLLGDPQKH |
| | 6xHis_v1 | DCSLFIRDAQKNDTGMYFFRVVREPFVRYSYKKSQLSLHVT |
| | | SLSRTPQILIPGALDPDHSKNLTCSVPWACEQGTPPIFSWMS |
| | | AAPTSLGLRTTHSSVLIITPRPQDHGTNLTCQVKFPGAGVTT |
| | | ERTIQLNVSYASQNPRTDIFLGDGSGRKARKQGVVQGSHHH |
| | | HHH |
| | | (SEQ ID NO: 237) |
| C33W49 | Human CD33 ECD | DPNFWLQVQESVTVQEGLCVLVPCTFFHPIPYYDKNSPVHG |
| | (D18-H259) from | YWFREGAIISRDSPVATNKLDQEVQEETQGRFRLLGDPSRN |
| | Uniprot P20138 | NCSLSIVDARRRDNGSYFFRMERGSTKYSYKSPQLSVHVTD |
| | with C-terminal | LTHRPKILIPGTLEPGHSKNLTCSVSWACEQGTPPIFSWLSAA |
| | 6xHis tag (SEQ ID | PTSLGPRTTHSSVLIITPRPQDHGTNLTCQVKFAGAGVTTER |
| | NO: 246) | TIQLNVTYVPQNPTTGIFPGDGSGKQETRAGVVHGSHHHHH |
| | | H |
| | | (SEQ ID NO: 238) |
| C33W50 | Cyno CD33 ECD | DPRVRLEVQESVTVQEGLCVLVPCTFFHPVPYHTRNSPVHG |
| | (D37-Q274) from | YWFREGAIVSLDSPVATNKLDQEVQEETQGRFRLLGDPSRN |
| | XP_005590138.1 | NCSLSIVDARRRDNGSYFFRMEKGSTKYSYKSTQLSVHVTD |
| | with C-terminal | LTHRPQILIPGALDPDHSKNLTCSVPWACEQGTPPIFSWMSA |
| | 6xHis tag (SEQ ID | APTSLGLRTTHSSVLIITPRPQDHGTNLTCQVKFPGAGVTTE |
| | NO: 246) | RTIQLNVSYASQNPRTDIFLGDGSGKQGVVQGSHHHHHH |
| | | (SEQ ID NO: 239) |
| C33W51 | Human CD33 ECD | DPNFWLQVQESVTVQEGLCVLVPCTFFHPIPYYDKNSPVHG |
| | (D18-G241) from | YWFREGAIISRDSPVATNKLDQEVQEETQGRFRLLGDPSRN |
| | Uniprot P20138 | NCSLSIVDARRRDNGSYFFRMERGSTKYSYKSPQLSVHVTD |
| | with 6xHis tag | LTHRPKILIPGTLEPGHSKNLTCSVSWACEQGTPPIFSWLSAA |
| | (SEQ ID NO: 246) | PTSLGPRTTHSSVLIITPRPQDHGTNLTCQVKFAGAGVTTER |
| | | TIQLNVTYVPQNPTTGHHHHHH |
| | | (SEQ ID NO: 240) |

### Example 2. Generation of Anti-CD33×82 Antibodies

Antibodies were generated using the Ablexis Transgenic mice technologies as follows. The AlivaMab mice were engineered to produce human/mouse immunoglobulins. AlivaMab transgenic mice were immunized with recombinant human CD33 protein (a selection of antigens from Table 7). Lymphocytes were extracted from secondary lymphoid organs and either fused with FO mouse myeloma cell line for hybridoma generation or subjected to single cell sorting via FACS. Hybridoma supernatants were screened by MSD electrochemiluminescence for binding to human embryonic kidney (HEK) cells over-expressing human CD33 ECD. The samples identified from the screening were further assayed with FACS for binding to HEK cells over-expressing human CD33 ECD (positive signal) compared to parental HEK cells (negative signal). Confirmed cell binders were light chain isotyped using ELISA. Single cell sorting supernatants were screened by MSD electrochemiluminescence for binding to recombinant human CD33 protein. Several hits with the desired binding profile were selected and sequenced.

V region cloning was performed as follows. Both RNA purified by the Qiagen® RNeasy Plus Mini Kit and B cell lysate were used to perform cDNA synthesis using the Smarter cDNA synthesis kit (Clontech, Mount View, CA). To facilitate cDNA synthesis, oligodT was used to prime reverse transcription of all messenger RNAs, followed by “5′ capping” with a Smarter IIA oligonucleotide. Subsequent amplification of the VH and VL fragments was performed using a 2-step PCR amplification using 5′ primers targeting the Smarter IIA cap and 3′ primers targeting consensus regions in CH1. Briefly, each 50 μl PCR reaction consists of 20 μM of forward and reverse primer mixes, 25 μl of PrimeStar Max DNA polymerase premix (Clontech), 2 μl of unpurified cDNA, and 21 μl of double-distilled H2O. The cycling program starts at 94° C. for 3 min, followed by 35 cycles (94° C. for 30 Sec, 55° C. for 1 min, 68° C. for 1 min), and ends at 72° C. for 7 min. A second round of PCR was performed using VL and VH second round primers that contained 15 bp complementary extensions that “overlap” respective regions in their respective Lonza mother vector (VH and VL). Second round PCR was performed with the following program: 94° C. for 3 min; 35 cycles (94° C. for 30 Sec, 55° C. for 1 min, 68° C. for 1 min), and ends at 72° C. for 7 min. In-Fusion® HD Cloning Kit (Clonetech, U.S.A.) was used for directional cloning of VL gene into Lonza hulgK or Lambda vector and VH gene into Lonza hulgG1 vector. To facilitate In-20) Fusion® HD Cloning, PCR products were treated with Cloning Enhancer before performing In-Fusion® HD Cloning. The cloning and transformation were performed according to manufacturer's protocol (Clonetech, U.S.A.). Mini-prep DNAs were subjected to Sanger sequencing to confirm that complete V-gene fragments were obtained.

The anti-CD33 antibodies were expressed in ExpiCHO-S™ cells (ThermoFisher Scientific; Waltham, MA, Cat #A29127) by transient transfection with purified plasmid DNA encoding the proteins following the manufacturer's recommendations. Briefly, ExpiCHO-S™ cells were maintained in suspension in ExpiCHO™ expression medium (ThermoFisher Scientific, Cat #A29100) in an orbital shaking incubator set at 37° C., 8% CO2 and 125 RPM. The cells were passaged and diluted prior to transfection to 6.0×106 cells per ml, maintaining cell viability at 99.0% or better. Transient transfections were done using the ExpiFectamine™ CHO transfection kit (ThermoFisher Scientific, Cat #A29131). For each ml of diluted cells to be transfected, 0.5 microgram of scFv Fc fusion encoding DNA and 0.5 microgram of pAdVAntage DNA (Promega, Cat #E1711) was used and diluted into OptiPRO™ SFM complexation medium. ExpiFectamine™ CHO reagent was used at a 1:4 ratio (v/v, DNA:reagent) and diluted into OptiPRO™. The diluted DNA and transfection reagent were combined for one minute, allowing DNA/lipid complex formation, and then added to the cells. After overnight incubation, ExpiCHO™ feed and ExpiFectamine™ CHO enhancers were added to the cells as per the manufacturer's Standard protocol. Cells were incubated with orbital shaking (125 rpm) at 370C for seven days prior to harvesting the culture broth. The culture supernatant from the transiently transfected ExpiCHO-S™ cells was clarified by centrifugation (30 min, 3000rcf) followed by filtration (0.2 μm PES membrane, Corning; Corning, NY).

Protein Purification was performed as follows. The filtered cell culture supernatant was loaded onto a pre-equilibrated (1×DPBS, pH 7.2) MabSelect Sure Protein A column (GE Healthcare) using an AKTAXpress chromatography system. After loading, the column was washed with 10 column volumes of 1×DPBS, pH 7.2. The protein was eluted with 10 column volumes of 0.1 M sodium (Na)-Acetate, pH 3.5. Protein fractions were neutralized immediately by the addition of 2.5 M Tris HCl, pH 7.5 to 20% (v/v) of the elution fraction volume. Peak fractions were pooled and filtered (0.2 μm). The quality of the purified protein was assessed by analytical size exclusion HPLC (Agilent HPLC system).

### Example 3. Generation of Bispecific CD33xVdelta2 Antibodies

Bispecific molecules were made using the knob-into-hole mutations to generate heterodimers of a CD33 binder fused to Fc and a VHH Vdelta2 binder fused to Fc. The Vdelta2 binding VHH sequences used were antibody 6H4 and antibody 5D3 described in WO2015156673. The 6H4 sequence is set forth in SEQ ID NO: 58. The 5D3 sequence is set forth in SEQ ID NO: 57.

The CD33 binding Fab was on the Hole Fc and the Vdelta2 binding VHH was on the Knob Fc. CD33 VH and human CH1 constant region were fused with hinge on Fc containing several mutations L234A/L235A/D265S_M252Y/S254T/T256E_T366S/L368A/Y407V_H435R/Y436F. The AAS mutations were introduced into the Fc portion of both heavy chains to render the Fc receptor silent. The YTE (M252Y/S254T/T256E) mutations were introduced into the Fc portion of both heavy chains of JL5 to increase half-life. The delta2 binding VHH was fused to hinge and Knob Fc including the following mutations: C220S_L234A/L235A/D265S_M252Y/S254T/T256E_T366W.

The RF mutations were introduced on the hole heavy chain to aid in purification.

The following Fab (CD33)xVHH (Vδ2) constructs were prepared:

CD33 antibody JL5 combined with Vdelta2 antibody 6H4 (JL5x6H4) corresponding to sequences SEQ ID NO: 101, 102 and 103.

CD33 antibody JL5 combined with Vdelta2 antibody 5D3 (JL5x5D3) corresponding to sequences SEQ ID NO: 104, 105 and 106.

CD33 antibody JL6 combined with Vdelta2 antibody 6H4 (JL6x6H4) corresponding to sequences SEQ ID NO: 107, 108 and 109; but without YTE mutations.

CD33 antibody JL6 combined with Vdelta2 antibody 5D3 (JL6x5D3) corresponding to sequences SEQ ID NO: 110, 111 and 112; but without YTE mutations.

CD33 antibody JL2 combined with Vdelta2 antibody 6H4 (JL2x6H4) corresponding to sequences SEQ ID NO: 113, 114 and 115; but without YTE mutations.

CD33 antibody JL2 combined with Vdelta2 antibody 5D3 (JL2x5D3) corresponding to sequences SEQ ID NO: 116, 117 and 118; but without YTE mutations.

CD33 antibody JL3 combined with Vdelta2 antibody 6H4 (JL3x6H4) corresponding to sequences SEQ ID NO: 119, 120 and 121; but without YTE mutations.

CD33 antibody JL3 combined with Vdelta2 antibody 5D3 (JL3x5D3) corresponding to sequences SEQ ID NO: 122, 123 and 124; but without YTE mutations.

The molecules were expressed in CHO cell line and purified by ProA capture followed by CH1 affinity capture. Briefly, the antibodies were initially purified by Mab Select SuRe Protein A column (GE Healthcare). The column was equilibrated with PBS pH 7.2 and loaded with fermentation supernatant at a flow rate of 2 mL/min. After loading, the column was washed with 4 column volumes of PBS followed by elution in 30 mM sodium acetate, pH 3.5. Fractions containing protein peaks as monitored by absorbance at 280 nm were pooled and neutralized to pH 5.0 by adding 1% 3 M sodium acetate pH 9.0. The antibodies were further purified by CH1 capture and eluted in histidine buffer.

### Example 4: Bispecific CD33xVδ2 Antibodies Bind CD33-Expressing Cells and Vγ9Vδ2 Cells

### Introduction

The ability of the bispecific CD33xVδ2 antibodies to bind to CD33-expressing cells and Vγ9Vδ2 cells was tested.

### Materials and Methods

### Cell Lines

The CD33-expressing acute monocytic leukemia (AML) cell line THP-1 (ECACC, Sigma) was cultured in RPMI 1640 ATCC mod (Gibco), 10% heat-inactivated FBS, 50 μg/ml Gentamycin and 2-mercaptoethanol. Purified Vγ9Vδ2-T cell lines were generated as described previously (de Bruin et al. (2017), Oncoimmunology 7 (1): e1375641). In short, Vδ2″-T cells were isolated from healthy donor (HD) PBMCs using FITC-conjugated anti-Vδ2 TCR (Beckman coulter, clone IMMU 389) in combination with anti-mouse IgG microbeads (Miltenyi Biotec) and cultured weekly with irradiated feeder mix consisting of PBMCs from 2 healthy donors, JY cells, IL-7 (10 U/mL), IL-15 (10 ng/mL, R&D Systems) and phytohaemagglutinin (50 ng/ml PHA; Thermo Fisher Scientific). Purity of Vγ9Vδ2-T cell lines was maintained at >90% and <5% CD4+.

### Bispecific Antibody Binding

To assess CD33 binding, THP-1 cells were incubated for 45-60 minutes at 4° C. with a concentration range of 316-0.00316 nM of bispecific antibody JL2x6H4, JL3x6H4, JL5x6H4, JL6x6H4, B21Mx6H4, JL2x5D3, JL3x5D3, JL5x5D3, JL6x5D3 or B21Mx5D3.

To assess Vδ2 binding, Vγ9Vδ2 cells were incubated for 45-60 minutes at 4° C. with a concentration range of 316-0.00316 nM of bispecific antibody JL2x6H4, JL3x6H4, JL5x6H4, JL6x6H4, B21Mx6H4, JL2x5D3, JL3x5D3, JL5x5D3, JL6x5D3 or B21Mx5D3 or with a bispecific antibody that binds gp120 and another irrelevant target.

Bound bispecific antibody was detected by incubation with an Alexa Fluor® 647 conjugated F(ab′)2 Goat anti-human IgG antibody (H+L) (Jackson) for 30 minutes at 4° C.

### FACS

Samples were measured by FACS Celesta (BD) and the data were analyzed using FlowJo software (FlowJo).

### Results

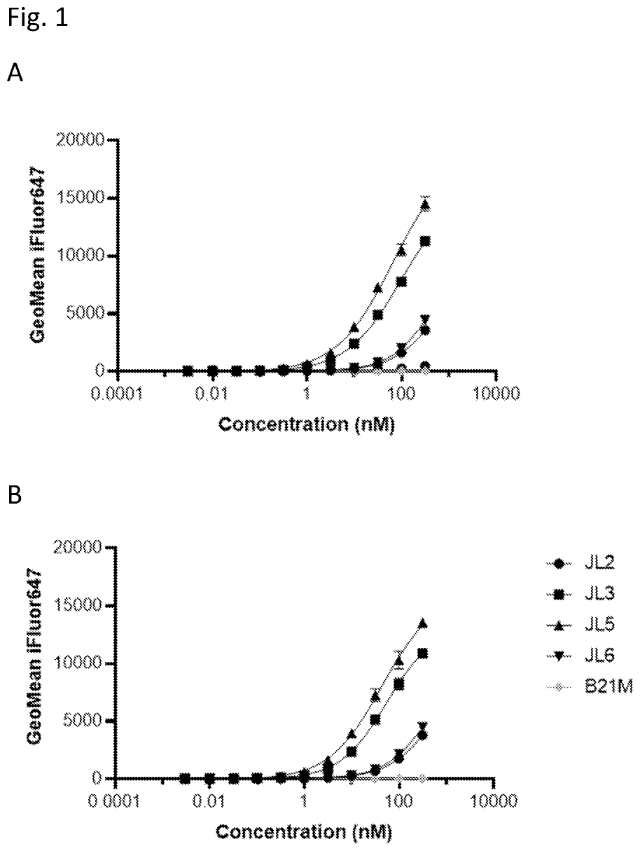

All bispecific CD33xVδ2 antibodies were found to bind CD33-expressing THP-1 cells (FIG. 1). As expected, negative control antibodies with a Respiratory syncytial virus (RSV) binding domain (B21M) instead of a CD33 binding domain did not bind to THP-1 cells.

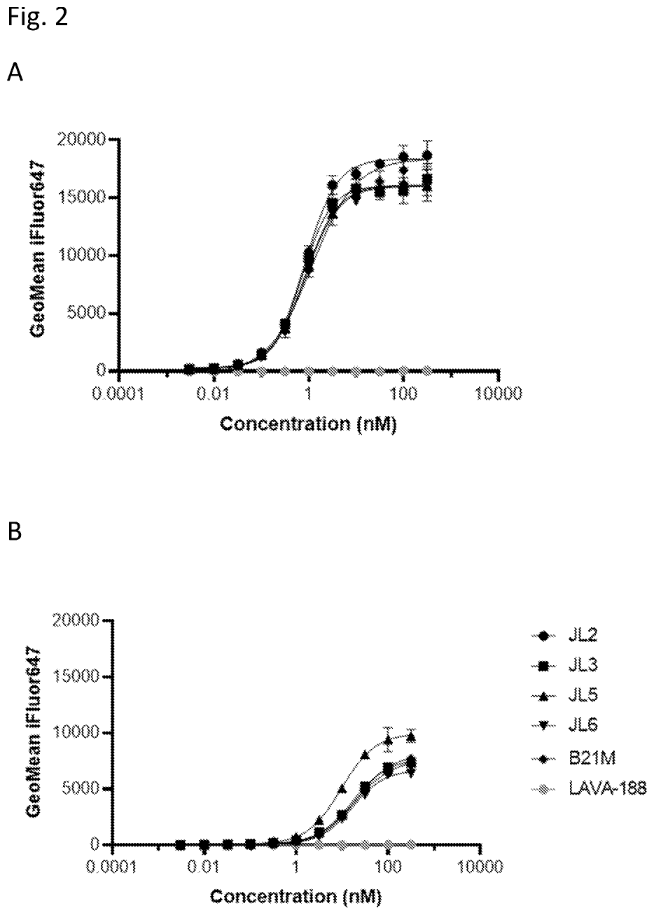

Furthermore, all bispecific CD33xVδ2 antibodies were found to bind Vγ9Vδ2 T cells (FIG. 2). As expected, the RSVxVδ2 antibody also bound Vγ9Vδ2 T cells, while a negative control antibody without a Vδ2 binding domain did not bind to these cells. Bispecific CD33xVδ2 antibodies that contained 6H4 as the Vδ2 binding domain bound with higher affinity to Vγ9Vδ2 T cells than bispecific antibodies that contained 5D3 as the Vδ2 binding domain.

### Example 5: Bispecific CD33xVδ2 Antibodies can Mediate Cytotoxicity Against CD33-Expressing Cells

### Introduction

As described in Example 4, bispecific CD33xVδ2 antibodies bind both CD33-expressing cells and Vγ9Vδ2-T cells. It was subsequently tested whether the bispecific antibodies could induce cytotoxicity towards CD33-expressing tumor cells.

### Materials and Methods

### Cell Lines

CD33-expressing THP-1 cells (target cells) and Vγ9Vδ2-T cells (effector cells) were grown as described in Example 4.

### Cytotoxicity Assay

THP-1 target cells were labeled with cell trace violet (CTV) and incubated at 37° C. in the presence of bispecific CD33xVδ2 antibodies or negative control antibodies (RSVxVδ2) and Vγ9Vδ2-T effector cells (E) at a 1:1 or 1:20 (E: T) ratio (2,500 effector cells and 50,000 target cells for the 1:20 ratio). An antibody concentration series of nine 5-fold dilutions starting at 5 nM was tested. After 22 and 94 hrs, the dead cells were stained using 7-Aminoactinomycin D (7AAD). THP-1 cell killing was determined by determining the percentage of CTV+ 7AAD-cells. EC50 was determined by non-linear regression using Prism software (GraphPad).

### Results

Killing of THP-1 cells was measured and EC50s were determined. No killing was observed in the presence of negative control antibodies. However, all bispecific CD33xVδ2 antibodies were able to mediate killing of THP-1 tumor cells. Generally, antibodies comprising a 6H4-based Vδ2 binding domain were more potent (lower EC50) than the antibodies comprising a 5D3-based binding domain (Table 8). Bispecific antibodies having a JL3 or JL5 CD33 binding domain were more potent than those comprising a JL2 or JL6 domain. Efficient killing was seen even at a 1:20 effector to target cell ratio.

TABLE 8

| | Cytotox EC50 (pM) | Cytotox EC50 (pM) |
|---|---|---|
| Antibody | E:T 1:1 - 22 hrs | E:T 1:20 - 94 hrs |
| JL2 × 6H4 | 4 | 20 |
| JL3 × 6H4 | 2.5 | 12 |
| JL5 × 6H4 | 5.4 | 15 |
| JL6 × 6H4 | 6.2 | 30 |
| JL2 × 5D3 | 28 | 380* |
| JL3 × 5D3 | 15 | 77 |
| JL5 × 5D3 | 12 | 45 |
| JL6 × 5D3 | 63 | 560* |

*Estimation, no plateau for the highest concentration

### Example 6: Bispecific CD33xVδ2 Antibodies Preferentially Induce Killing of Tumor Cells Over Healthy Cells

### Introduction

As described in Example 5, bispecific CD33xVδ2 antibodies can induce cytotoxicity towards CD33-expressing tumor cells. It was subsequently tested whether cytotoxicity could also be obtained using PBMCs from fresh blood as effector cells and whether the CD33xVδ2 antibodies also induce cytotoxicity towards healthy CD33 positive, CD14+ cells.

### Materials and Methods

### Cell Lines

CD33-expressing THP-1 cells (target cells) were grown as described in Example 5. Whole heparin-treated blood from healthy donor volunteers were obtained from blood supply service Sanquin and used for isolation of peripheral blood mononuclear cells (PBMC). PBMC were isolated using Lymphoprep™ density gradient centrifugation.

To determine the percentage of Vγ9Vδ2-T cells, PBMC were stained with PerCP-Cy5.5 labeled anti-CD3 mAb (Biolegend clone SK7), PE labeled anti-TCR Vγ9 mAb (Biolegend clone B6) and FITC labeled anti-TCR Vδ2 mAb (Beckman Coulter clone IM1464). CD33 expression on THP-1 and CD14+ monocyte cells was determined using PE-Cy7 labeled anti-CD14 mAb (Biolegend clone 63D3), APC labeled anti-CD33 mAb (Biolegend clone WM53).

### Cytotoxicity Assay

THP-1 target cells were labeled with cell trace violet (CTV) and incubated at 37° C. in the presence of bispecific CD33xVδ2 antibodies or negative control antibodies (RSVxVδ2) and PBMC effector cells (E) in a 5:1 (E: T) ratio (250,000 effector cells and 50,000 target cells). An antibody concentration series of six 5-fold dilutions starting at 5 nM was tested. After 94 hrs, the cells were stained with PE-Cy7 labeled anti-CD14 mAb and 7AAD. THP-1 cell killing was determined based on percentage of CTV+7AAD-cells, whereas monocyte killing was determined based on the percentage of CTV-CD14+7AAD-cells.

### Results

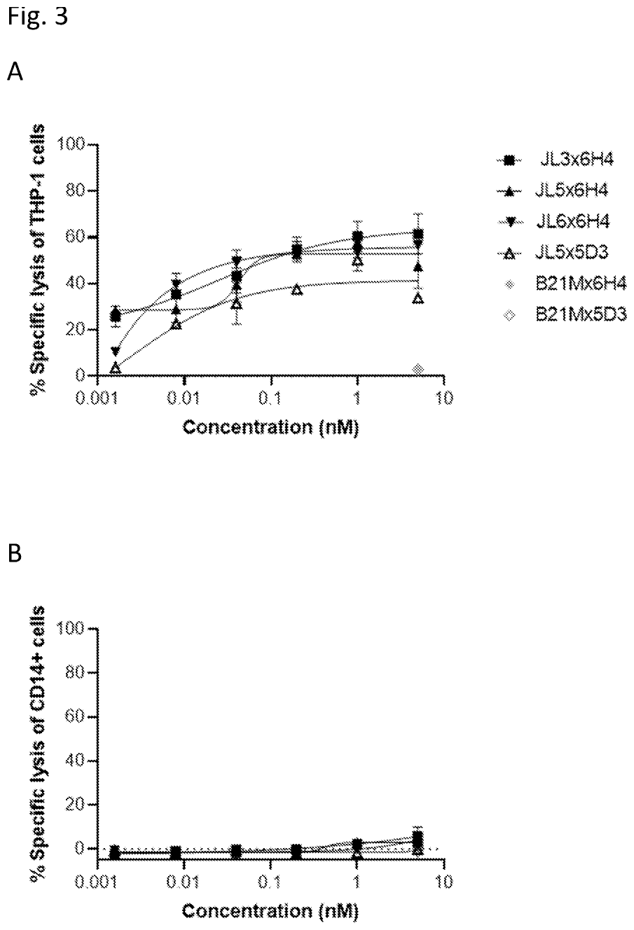

Killing of target cells is shown in FIG. 3. It was found that PBMCs (containing 1.84% of Vγ9+Vδ2+T cells of total PBMC) were able to mediate killing of THP-1 tumor cells in the presence of bispecific CD33xVδ2 antibodies (panel A). On the other hand, almost no lysis occurred of healthy CD14+ target cells (panel B). This indicates that bispecific CD33xVδ2 antibodies preferentially mediate killing of tumor cells over healthy cells. This difference does not appear to be due to a difference in CD33 expression between THP-1 target cells and CD14+ target cells, because CD33 expression was found to be about twice as high on CD14+ cells compared with THP-1 cells.

### Example 7: Bispecific CD33xVδ2 Antibodies Induce Proliferation of Vγ9Vδ2 T Cells

### Introduction

It was subsequently tested whether bispecific CD33xVδ2 antibodies induce proliferation of Vγ9Vδ2 T cells in the presence of CD33 positive target cells.

### Materials and Methods

### Cell Lines

CD33-expressing THP-1 cells (target cells) were grown as described in Example 4. Vγ9Vδ2 T cells (effector cells) from two different donors were cultured as described in Example 4.

### Proliferation Assay

THP-1 target cells were labeled with cell trace violet (CTV). Vγ9Vδ2 effector cells were labeled with cell trace far red (CTFR) in the presence of 50 IU/mL IL-2. Target cells and effector cells were co-incubated at 37° C., 5% CO2 (200 μl/well) at a 1:20 effector cell to target cell ratio (50,000 target cells, 2,500 effector cells) in the presence of 1 nM bispecific antibody. 123count eBeads™ Counting Beads (Invitrogen) were used to assess the number of cells on day 0, day 1, day 4, day 7, day 11 and day 14.

### Results

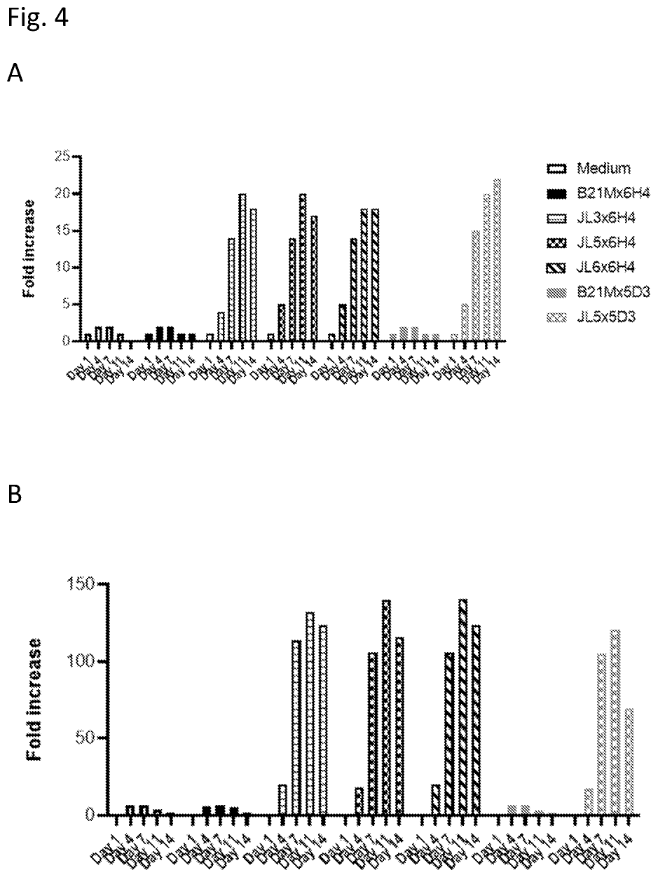

Proliferation of Vγ9Vδ2 effector cells is shown in FIG. 4. It was found that all bispecific CD33xVδ2 antibodies were able to induce proliferation of Vγ9Vδ2 effector cells from two different donors. No proliferation was seen in the presence of bispecific RSVxVδ2 antibodies, indicating that the proliferation was dependent on the presence of the target cells.

### Example 8: Bispecific scFv CD33xVδ2 Antibodies can Mediate Cytotoxicity Against CD33-Expressing Cells

### Introduction

It was tested whether the bispecific antibodies could induce cytotoxicity towards CD33-expressing tumor cells when the CD33 binding arm was provided in an scFv format.

### Materials and Methods

### Cell Lines

CD33-expressing THP-1 cells (target cells) and Vγ9Vδ2-T cells (effector cells) from two different donors (Donor 104 and Donor 156) were grown as described in Example 5.

Production of scFv-VHH Bispecific Molecules

scFv-VHH bispecific molecules were generated as follows: A fixed design of scFv (VL-L-VH)-L-VHH with a “bird” linker between VL and VH, a fixed short 5 amino acid linker (GGGGS (SEQ ID NO: 100)) between scFv and VHH and C-tag at the C terminal protein was used. Amino acid sequences of bispecific scFv VHH molecules were reverse-translated to cDNA and then codon-optimized for expression in human cells. Regulatory elements were added: an N-terminal Kozak sequence and C-terminal stop codon and the cDNA was made as a synthetic gene. cDNAs were cloned into a suitable vector and their sequences were verified. Expression of the proteins was performed by transient transfection of the resulting plasmids in HEK293_E cells. Proteins were purified from the culture supernatant by means of C-tag affinity chromatography and gel filtration.

| SEQ | | | |
|---|---|---|---|
| ID. | code | Description | Sequence |
| 54 | JL2 VL | Light chain | DIVMTQSPLSLPVTPGEPASISCRSSQSLLH |
| | | variable | SDGYNYLDWYLQKSGQSPQLLIYLGSYRAS |
| | | region of | GVPDRFSGSGSGTDFTLKISRVEAEDVGIY |
| | | JL2 | YCMQVLQTPWTFGQGTKVEIK |
| 67 | Bird | | GGSEGKSSGSGSESKSTGGS |
| | linker | | |
| 53 | JL2 VH | Heavy chain | EVOLVESGGGLVQPGGSLRLSCVVSGFTFS |
| | | variable | SYWMTWVRQAPGKGLEWVANIKQDGSER |
| | | region of | YYVDSVKGRFTISRDSAKNSLYLQMNSLR |
| | | JL2 | AEDTAVYYCAREVGYNWNQGGYFDYWG |
| | | | QGTLVTVSS |
| 100 | G4S | Linker | GGGGS |
| | linker | | |
| 58 | 6H4 | VHH | EVQLVESGGGLVQAGGSLRLSCAASGRPFS |
| | | | NYGMGWFRQAPGKKREFVAGISWSGGST |
| | | | DYADSVKGRFTISRDNAKNTVYLQMNSLK |
| | | | PEDTAVYYCAAVFSGAETAYYPSDDYDY |
| | | | WGQGTQVTVSS |
| 245 | C-tag | tag | AAAEPEA |
| 56 | JL3 VL | Light chain | QSVLTQPPSASGTPGQRVTISCSGSSSNIGS |
| | | variable | NIVNWYQQFPGTAPKLLLYSDNQRPSGVP |
| | | region of | DRFSGSKSGTSASLAISGLQSEDEADYYCA |
| | | JL3 | AWDDSLNGPVFGTGTKVTVL |
| 55 | JL3 VH | Heavy chain | QVQLQESGPGLVKPSETLSLTCSVSGGSIRN |
| | | variable | YYWSWIRQSAGKELEWFGHIFSTGHINYDS |
| | | region of | SLKSRVTMSVDTSNNQFSLKLRSVTAADT |
| | | JL3 | AVYYCARDNGAALFDFWGQGTLVTVSS |
| 50 | JL5 VL | Light chain | QSVLTQPPSASGTPGQRVTISCSGSSSNIGS |
| | | variable | NIVNWYQQFPGTAPKLLIYSNNQRPSGVPD |
| | | region of | RVSGSKSGTSASLAISGLQSEDEADYYCAA |
| | | JL5 | WDDSLNGPVFGPGTKVTVL |
| 49 | JL5 VH | Heavy chain | QVQLQESGPGLVKPSETLSLTCSVSGASIRN |
| | | variable | YYWSWIRQTAGKGLEWLGHIYSTGNIHYN |
| | | region of | PSLKSRVTMSVDTSNNQFSLKLRSVTAADT |
| | | JL5 | AVYYCARDNGAALFDYWGQGTLVTVSS |
| 52 | JL6 VL | Light chain | QSVLTQPPSASGTPGQRVTISCSGSSSNIGS |
| | | variable | NPVNWYQQLPGTAPKLLIYSNNQRPSGVP |
| | | region of | DRFSGSKSGTSASLAISGLQSEDEADYFCA |
| | | JL6 | AWDDSLNGPVFGGGTKLTVL |
| 51 | JL6 VH | Heavy chain | QVQLQESGPGLVKPSETLSLTCTVSGGSISS |
| | | variable | YYWGWIRQPPGKGLEWIGYIYYSGSTNYN |
| | | region of | PSLKSRVTISVDTSKNQFSLKLSSVTAADTA |
| | | JL6 | VYYCARMWEILGFDPWGQGTLVTVSS |
| 57 | 5D3 | VHH | EVOLVESGGGLVQAGGSLRLSCAASGRPFS |
| | | | NYAMGWFRQAPGKEREFVTVISWSGGSTY |
| | | | YADSVKGRFTISRDNAKNTVYLQMNSLKP |
| | | | EDTAVYYCAAQFSGASTVVAGTALDYDY |
| | | | WGQGTRVTVSS |
| 241 | JL1 | VH | EVOLVESGGGLVQPGGSLKVSCEASGFTFS |
| | | | VSAIHWVRQASGKGLEWIGRIRSKGNSYA |
| | | | TAYAASVKGRFTISRDDSKNTAYLQMDSL |
| | | | KTEDTAVYYCTRHNDKWNYYGLDVWGQ |
| | | | GTTVTVSS |
| 242 | JL1 | VL | DIVMTQSPLSLPVTPGEPASISCKSSQSLLF |
| | | | SNGYKFLDWYLQRPGQSPQLLIYLGSYRAS |
| | | | GVPDRFSGSGSGTDFTLKISRVEAEDVGLY |
| | | | YCMQALQTPPTFGGGTKVEIK |
| 243 | JL4 | VH | QVQLVESGGGLVQPGGSLRLSCAASGITFS |
| | | | NYWMSWVRQAPGKGLEWVASIKRDGSDK |
| | | | YYVDSVKGRFTISRDNAKNSLSLQMHSLR |
| | | | AEDTAVYYCAKGEFDYWGQGTLVTVSS |
| 244 | JL4 | VL | DVVMTQSPLSLPVTLGQPASISCRSSQSLVY |
| | | | SDGNTYLNWFQQRPGQSPRRLIYKVSTRDS |
| | | | GVPDRFSGSGSGTDFTLKISRVEAEDVAVY |
| | | | YCLQGTHWPWTFGQGTKVEIK |

### Cytotoxicity Assay

THP-1 target cells were labeled with cell trace violet (CTV) and incubated at 37° C. in the presence of bispecific CD33xVδ2 antibodies or negative control antibodies (RSVxVδ2) and Vγ9Vδ2-T effector cells (E) at a 1:1 (E: T) ratio (50,000 effector cells and 50,000 target cells). An antibody concentration series of 10 half-log dilutions starting at 3.16 nM was tested. After 24 hrs, THP-1 cell killing was determined by determining the percentage of 7AAD-CTV+ cells using flow cytometry.

### Results

Killing of THP-1 cells was measured and EC50 values were determined. No killing was observed in the presence of negative control antibodies. All bispecific scFv CD33xVδ2 antibodies were able to mediate killing of THP-1 tumor cells.

| | Cytotox EC50 (nM) | Cytotox EC50 (nM) |
|---|---|---|
| Antibody | Donor 104 | Donor 156 |
| JL1 × 6H4 | 0.011 | 0.0098 |
| JL2 × 6H4 | 0.00097* | 0.00077* |
| JL3 × 6H4 | 0.00079* | 0.00099* |
| JL4 × 6H4 | 0.00040* | 0.00035* |
| JL5 × 6H4 | 0.0013* | 0.0019* |
| JL6 × 6H4 | 0.0012 | 0.0013 |
| JL1 × 5D3 | 0.61# | 0.43# |
| JL2 × 5D3 | 0.0027 | 0.0019* |
| JL3 × 5D3 | 0.0013* | 0.0008* |
| JL4 × 5D3 | 0.027# | 0.023 |
| JL5 × 5D3 | 0.00093* | 0.0007* |
| JL6 × 5D3 | 0.0044 | 0.0029 |

*#Estimation, no plateau for the highest or lowest concentration

1. An isolated multispecific antibody comprising a first antigen-binding region capable of binding human CD33 and a second antigen-binding region capable of binding a human Vγ9Vδ2 T cell receptor:
wherein the first antigen-binding region comprises the HCDR1, the HCDR2, the HCDR3, the LCDR1, the LCDR2 and the LCDR3 of:
a) SEQ ID NOs: 1, 2, 3, 4, 5, and 6, respectively;
a) SEQ ID NOs: 7, 8, 9, 10, 11, and 12, respectively:
d) SEQ ID NOs: 13, 14, 15, 16, 17 and 18, respectively; or
e) SEQ ID NOs: 19, 20, 21, 22, 23 and 24, respectively; and
wherein the second antigen-binding region binds the Vδ2 chain of the Vγ9Vδ2 T cell receptor.

2. The isolated multispecific antibody of claim 1, wherein the second antigen-binding region is a single-domain antibody and comprises the CDR1, CDR2 and CDR3 of:
a) SEQ ID NOs: 28, 29, and 30, respectively:
b) SEQ ID NOs: 25, 26, and 27, respectively:
c) SEQ ID NOs: 31, 32, and 33, respectively:
d) SEQ ID NOs: 34, 35, and 36, respectively
e) SEQ ID NOs: 37, 38, and 39, respectively;
f) SEQ ID NOs: 40, 41, and 42, respectively:
g) SEQ ID NOs: 43, 44, and 45, respectively; or
h) SEQ ID NOs: 46, 47, and 48, respectively.

3. The isolated multispecific antibody of claim 2, wherein the first antigen-binding region comprises the HCDR1, the HCDR2, the HCDR3, the LCDR1, the LCDR2 and the LCDR3 of SEQ ID NOs: 1, 2, 3, 4, 5 and 6, respectively.

4. The isolated multispecific antibody of claim 2, wherein the first antigen-binding region comprises the HCDR1, the HCDR2, the HCDR3, the LCDR1, the LCDR2 and the LCDR3 of SEQ ID NOs: 1, 2, 3, 4, 5 and 6, respectively, and the second antigen-binding region is a single-domain antibody and comprises the CDR1, CDR2 and CDR3 of: SEQ ID NOs: 28, 29, and 30, respectively.

5. The isolated multispecific antibody of claim 2, wherein the first antigen-binding region comprises the HCDR1, the HCDR2, the HCDR3, the LCDR1, the LCDR2 and the LCDR3 of SEQ ID NOs: 1, 2, 3, 4, 5 and 6, respectively, and the second antigen-binding region is a single-domain antibody and comprises the CDR1, CDR2 and CD3 of: SEQ ID NOs: 25, 26, and 27, respectively.

6. The isolated multispecific antibody of claim 1, wherein the second antigen-binding region is a single-domain antibody and comprises the CDR1, CDR2 and CD3 of: SEQ ID NOs: 28, 29, and 30, respectively.

7. The isolated multispecific antibody of claim 1,
wherein the first antigen-binding region comprises or consists of:
a) a sequence having at least 90%, 92%, 94%, 96%, 98%, or 100% sequence identity to the VH sequence of SEQ ID NO: 51, and a sequence having at least 90%, 92%, 94%, 96%, 98%, or 100% sequence identity to the VL sequence of SEQ ID NO: 52:
b) a sequence having at least 90%, 92%, 94%, 96%, 98%, or 100% sequence identity to the VH sequence of SEQ ID NO: 49, and a sequence having at least 90%, 92%, 94%, 96%, 98%, or 100% sequence identity to the VL sequence of SEQ ID NO: 50:
d) a sequence having at least 90%, 92%, 94%, 96%, 98%, or 100% sequence identity to the VH sequence of SEQ ID NO: 53, and a sequence having at least 90%, 92%, 94%, 96%, 98%, or 100% sequence identity to the VL sequence of SEQ ID NO: 54:
e) a sequence having at least 90%, 92%, 94%, 96%, 98%, or 100% sequence identity to the VH sequence of SEQ ID NO: 55, and a sequence having at least 90%, 92%, 94%, 96%, 98%, or 100% sequence identity to the VL sequence of SEQ ID NO: 56; or
and
wherein the second antigen-binding region comprises or consists of:
a) a sequence having at least 90%, 92%, 94%, 96%, 98%, or 100% sequence identity to SEQ ID NO: 58:
b) a sequence having at least 90%, 92%, 94%, 96%, 98%, or 100% sequence identity to SEQ ID NO: 57:
c) a sequence having at least 90%, 92%, 94%, 96%, 98%, or 100% sequence identity to SEQ ID NO: 59:
d) a sequence having at least 90%, 92%, 94%, 96%, 98%, or 100% sequence identity to SEQ ID NO: 60;
e) a sequence having at least 90%, 92%, 94%, 96%, 98%, or 100% sequence identity to SEQ ID NO: 61:
f) a sequence having at least 90%, 92%, 94%, 96%, 98%, or 100% sequence identity to SEQ ID NO: 62:
g) a sequence having at least 90%, 92%, 94%, 96%, 98%, or 100% sequence identity to SEQ ID NO: 63:
h) a sequence having at least 90%, 92%, 94%, 96%, 98%, or 100% sequence identity to SEQ ID NO: 64:
i) a sequence having at least 90%, 92%, 94%, 96%, 98%, or 100% sequence identity to SEQ ID NO: 65; or
j) a sequence having at least 90%, 92%, 94%, 96%, 98%, or 100% sequence identity to SEQ ID NO: 66.

8. The isolated multispecific antibody of claim 1, wherein
a) the isolated multispecific antibody comprises a Fab, an scFv, a (scFv)₂, a Fv, a F(ab′)₂ or a Fd wherein said Fab, scFv, (scFv)₂, Fv, F(ab′)₂ or Fd comprises the first antigen-binding region capable of binding human CD33 or,
b) the isolated multispecific antibody comprises:
a Fab comprising the first antigen-binding region capable of binding human CD33, and
a single-domain antibody comprising the second antigen-binding region capable of binding a human Vγ9Vδ2 T cell receptor: or,
c) the isolated multispecific antibody comprises:
an scFv comprising the first antigen-binding region capable of binding human CD33, and
a single-domain antibody comprising the second antigen-binding region capable of binding a human Vγ9Vδ2 T cell receptor.

9. The isolated multispecific antibody of claim 8, wherein the isolated multispecific antibody comprises an scFv comprising the first antigen-binding region capable of binding human CD33, and a VHH comprising the second antigen-binding region capable of binding a human Vγ9Vδ2 T cell receptor, and wherein the scFv comprises a peptide linker, optionally selected from the group of linkers set forth in SEQ ID NO: 67 to 99.

10. The isolated multispecific antibody of claim 1, wherein the first antigen-binding region and second antigen-binding region are directly or indirectly covalently linked via a peptide linker: optionally wherein the peptide linker the linker set forth in SEQ ID NO: 100.

11. The isolated multispecific antibody of claim 10, wherein the first antigen-binding region is located N-terminally of the second antigen-binding region.

12. The isolated multispecific antibody of claim 1, further comprising an Fc region consisting of a first Fc polypeptide and a second Fc polypeptide.

13. The isolated multispecific antibody of claim 1, wherein the isolated multispecific antibody comprises a Fab comprising the first antigen-binding region capable of binding human CD33, a VHH comprising the second antigen-binding region capable of binding a human Vγ9Vδ2 T cell receptor, and wherein the isolated multispecific antibody comprises an Fc region.

14. The isolated multispecific antibody of claim 1, comprising an Ig constant region or a fragment thereof selected from the group consisting of an IgG1, an IgG2, an IgG3, or an IgG4 isotype.

15. The isolated multispecific antibody of claim 12, wherein the Fc region is inert.

16. The isolated multispecific antibody of claim 15, wherein the Fc region, in one or both of the first and second Fc polypeptides, comprises an Ala at a position corresponding to 234, an Ala at a position corresponding to 235, and a Ser at a position corresponding to 265, wherein the numbering is according to Eu.

17. The isolated multispecific antibody of claim 12, wherein the first Fc polypeptide comprises a Trp at a position corresponding to 366 and the second Fc polypeptide comprises a Ser at a position corresponding to 366, an Ala at a position corresponding to 368 and a Val at a position corresponding to 407, or vice versa, wherein the numbering is according to Eu.

18. The isolated multispecific antibody of claim 12, wherein the Fc region, in one or both of the first and second Fc polypeptides, comprises a Tyr at a position corresponding to 252, a Thr at a position corresponding to 254, and a Glu at a position corresponding to 256, wherein the numbering is according to Eu.

19. The isolated multispecific antibody of claim 12, wherein the Fc region, in one of the first and second Fc polypeptides, comprises an Arg at a position corresponding to 435, and a Phe at a position corresponding to 436, wherein the numbering is according to Eu.

20. The isolated multispecific antibody of claim 1, wherein the multispecific antibody is a bispecific antibody.

21. The isolated multispecific antibody of claim 1, comprising:
a) the polypeptides set forth in SEQ ID NO: 101, 102 and 103:
b) the polypeptides set forth in SEQ ID NO: 104, 105 and 106;
c) the polypeptides set forth in SEQ ID NO: 107, 108 and 109:
d) the polypeptides set forth in SEQ ID NO:110, 111 and 112;
e) the polypeptides set forth in SEQ ID NO:113, 114 and 115:
f) the polypeptides set forth in SEQ ID NO:116, 117 and 118:
g) the polypeptides set forth in SEQ ID NO:119, 120 and 121: or
h) the polypeptides set forth in SEQ ID NO: 122, 123 and 124.

22. A pharmaceutical composition comprising an isolated multispecific antibody of claim 1 and a pharmaceutically acceptable carrier.

23. (canceled)

24. (canceled)

25. A nucleic acid construct, or a combination of nucleic acid constructs encoding the multispecific antibody of claim 1.

26. An expression vector comprising the nucleic acid construct or combination of nucleic acid constructs of claim 25.

27. An isolated host cell comprising the nucleic acid construct or combination of nucleic acid constructs of claim 25.

28. A method of treating a disease comprising administration of the multispecific antibody of claim 1 to a human subject in need thereof.

29. The method of claim 28, wherein the disease is a hematologic cancer; optionally wherein the hematologic cancer is selected from the group consisting of leukemia, lymphoma, multiple myeloma, acute myeloid leukemia (AML), myelodysplastic syndrome (MDS), acute lymphocytic leukemia (ALL), diffuse large B-cell lymphoma (DLBCL), chronic myeloid leukemia (CML), blastic plasmacytoid dendritic cell neoplasm (DPDCN), myeloproliferative neoplasm (MPNs), and mixed phenotype acute leukemia.
